SEQUENCE LISTING (41 sequences)

SEQ ID NO: 1 - protein, 162 aa, Homo sapiens
  REGION 1..162: note = hIL-15 (UniProt P40933)
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Homo sapiens
  REGION 1..114: note = mature hIL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 114 aa, synthetic construct
  REGION 1..114: note = Control IL-15
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 4 - protein, 114 aa, synthetic construct
  REGION 1..114: note = V3I
NWINVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 114 aa, synthetic construct
  REGION 1..114: note = V3W
NWWNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 114 aa, synthetic construct
  REGION 1..114: note = N4H
NWVHVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 114 aa, synthetic construct
  REGION 1..114: note = I6L
NWVNVLSDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 114 aa, synthetic construct
  REGION 1..114: note = K10R
NWVNVISDLRKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 9 - protein, 114 aa, synthetic construct
  REGION 1..114: note = K10Y
NWVNVISDLYKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 10 - protein, 114 aa, synthetic construct
  REGION 1..114: note = K11E
NWVNVISDLKEIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 114 aa, synthetic construct
  REGION 1..114: note = K11Y
NWVNVISDLKYIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 12 - protein, 114 aa, synthetic construct
  REGION 1..114: note = A23P
NWVNVISDLKKIEDLIQSMHIDPTLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - protein, 114 aa, synthetic construct
  REGION 1..114: note = T24P
NWVNVISDLKKIEDLIQSMHIDAPLYTESDVHPSCKVTAMKCFLLELQVISLESGDASIHDTVENLIILANNSLSSNGNVTESGCKECEELEEKNIKEFLQSFVHIVQMFINTS

SEQ ID NO: 14 - protein, 114 aa, synthetic construct
  REGION 1..114: note = L45I
NWVNVISDLKKIEDLIQSMHIDATLYTESDVHPSCKVTAMKCFLIELQVISLESGDASIHDTVENLIILANNSLSSNGNVTESGCKECEELEEKNIKEFLQSFVHIVQMFINTS

SEQ ID NO: 15 - protein, 114 aa, synthetic construct
  REGION 1..114: note = E46R
NWVNVISDLKKIEDLIQSMHIDATLYTESDVHPSCKVTAMKCFLLRLQVISLESGDASIHDTVENLIILANNSLSSNGNVTESGCKECEELEEKNIKEFLQSFVHIVQMFINTS

SEQ ID NO: 16 - protein, 114 aa, synthetic construct
  REGION 1..114: note = V49W
NWVNVISDLKKIEDLIQSMHIDATLYTESDVHPSCKVTAMKCFLLELQWISLESGDASIHDTVENLIILANNSLSSNGNVTESGCKECEELEEKNIKEFLQSFVHIVQMFINTS

SEQ ID NO: 17 - protein, 114 aa, synthetic construct
  REGION 1..114: note = A57P
NWVNVISDLKKIEDLIQSMHIDATLYTESDVHPSCKVTAMKCFLLELQVISLESGDPSIHDTVENLIILANNSLSSNGNVTESGCKECEELEEKNIKEFLQSFVHIVQMFINTS

SEQ ID NO: 18 - protein, 114 aa, synthetic construct
  REGION 1..114: note = S58P
NWVNVISDLKKIEDLIQSMHIDATLYTESDVHPSCKVTAMKCFLLELQVISLESGDAPIHDTVENLIILANNSLSSNGNVTESGCKECEELEEKNIKEFLQSFVHIVQMFINTS

SEQ ID NO: 19 - protein, 114 aa, synthetic construct
  REGION 1..114: note = N72T
NWVNVISDLKKIEDLIQSMHIDATLYTESDVHPSCKVTAMKCFLLELQVISLESGDASIHDTVENLIILANTSLSSNGNVTESGCKECEELEEKNIKEFLQSFVHIVQMFINTS

SEQ ID NO: 20 - protein, 114 aa, synthetic construct
  REGION 1..114: note = H105D
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVDIVQMFINTS

SEQ ID NO: 21 - protein, 114 aa, synthetic construct
  REGION 1..114: note = H105E
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVEIVQMFINTS

SEQ ID NO: 22 - protein, 114 aa, synthetic construct
  REGION 1..114: note = H105W
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVWIVQMFINTS

SEQ ID NO: 23 - protein, 114 aa, synthetic construct
  REGION 1..114: note = N112F
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIFTS

SEQ ID NO: 24 - protein, 114 aa, synthetic construct
  REGION 1..114: note = N112L
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFILTS

SEQ ID NO: 25 - protein, 114 aa, synthetic construct
  REGION 1..114: note = N112Y
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIYTS

SEQ ID NO: 26 - protein, 114 aa, synthetic construct
  REGION 1..114: note = E13C/L100C
NWVNVISDLKKICDLIQSMHIDATLYTESDVHPSCKVTAMKCFLLELQVISLESGDASIHDTVENLIILANNSLSSNGNVTESGCKECEELEEKNIKEFCQSFVHIVQMFINTS

SEQ ID NO: 27 - protein, 114 aa, synthetic construct
  REGION 1..114: note = L15C/I59C
NWVNVISDLKKIEDCIQSMHIDATLYTESDVHPSCKVTAMKCFLLELQVISLESGDASCHDTVENLIILANNSLSSNGNVTESGCKECEELEEKNIKEFLQSFVHIVQMFINTS

SEQ ID NO: 28 - protein, 114 aa, synthetic construct
  REGION 1..114: note = T24C/E93C
NWVNVISDLKKIEDLIQSMHIDACLYTESDVHPSCKVTAMKCFLLELQVISLESGDASIHDTVENLIILANNSLSSNGNVTESGCKECEELECKNIKEFLQSFVHIVQMFINTS

SEQ ID NO: 29 - protein, 114 aa, synthetic construct
  REGION 1..114: note = S29C/S102C
NWVNVISDLKKIEDLIQSMHIDATLYTECDVHPSCKVTAMKCFLLELQVISLESGDASIHDTVENLIILANNSLSSNGNVTESGCKECEELEEKNIKEFLQCFVHIVQMFINTS

SEQ ID NO: 30 - protein, 114 aa, synthetic construct
  REGION 1..114: note = eIL-15-A
NWVNVISDLRYIESLISSMNIDACLYTESDVHPKCSVTAMLCFLLELQVISLESGDPSIHDTVENLIILANNSLSSNGNKTEDGCKECEELECKNIKEFLQSFVHIVQMFINTS

SEQ ID NO: 31 - protein, 114 aa, synthetic construct
  REGION 1..114: note = eIL-15-B
NWVNVISDLRYIEDCISNMHIDAPLYTESDVHPKCSVTAMLCFLLELQVISLESGDPSCHDTVENLIILANNSLSSNGNVTESGCKECEELEEKNIKEFLQSFVWIVQMFILTS

SEQ ID NO: 32 - protein, 114 aa, synthetic construct
  REGION 1..114: note = eIL-15-C
NWVNVISDLREIESLIQNMHIDALLYTECDVHPKCKVTAMKCFLLELQVISLESGDAPIHDTVENLIILANNSLSSNGNKTEDGCKECEELEEKNIKEFLQCFVWIVQMFILTS

SEQ ID NO: 33 - protein, 114 aa, synthetic construct
  REGION 1..114: note = eIL-15-D
NWVNVISDLREIESLISSMNIDACLYTESDVHPKCSVTAMKCFLLELQVISLESGDASIHDTVENLIILANNSLSSNGNKTEDGCKECEELECKNIAEFLQSFVHIVQMFINTS

SEQ ID NO: 34 - protein, 114 aa, synthetic construct
  REGION 1..114: note = eIL-15-E
NWVNVISDLREIESLISSMNIDACLYTESDVHPKCSVTAMKCFLLELQVISLESGDASIHDTVENLIILANNSLSSNGNKTEDGCKECEELECKNIKEFLQSFVWIVQMFINTS

SEQ ID NO: 35 - protein, 114 aa, synthetic construct
  REGION 1..114: note = eIL-15-F
NWVNVISDLREIESLISSMHIDACLYTESDVHPKCSVTAMKCFLLELQVISLESGDASIHDTVENLIILANNSLSSNGNKTEDGCKECEELECKNIAEFLQSFVWIVQMFINTS

SEQ ID NO: 36 - protein, 114 aa, synthetic construct
  REGION 1..114: note = eIL-15-G
NWVNVISDLREIENLISSMNIDACLYTESDVHPSCKVTAMKCFLLELQVISLESGDAQIHDTVENLIILANNSLSSNGNYTEDGCKECEELECKNIAEFLQSFVWIVQMFINTS

SEQ ID NO: 37 - protein, 114 aa, synthetic construct
  REGION 1..114: note = eIL-15-H
NWVNVISDLREIENLIQSMNIDACLYTESDVHPKCSVTAMKCFLLELQVISLESGDASIHDTVENLIILANNSLSSNGNKTEDGCKECEELECKNIAEFLQSFVWIVQMFINTS

SEQ ID NO: 38 - protein, 114 aa, synthetic construct
  REGION 1..114: note = eIL-15-I
NWVNVISDLRYIESLISSMNIDACLYTESDVHPSCKVTAMKCFLLELQVISRESGDASIHDTVENLIILANNSLSSNGNKTEDGCKECEELECKNIAEFLQSFVHIVQMFILTS

SEQ ID NO: 39 - protein, 114 aa, synthetic construct
  REGION 1..114: note = eIL-15-J
NWVDVISDLREIENLISSMHIDACLYTESDVNPRCSVTAMKCFLLELQVISLESGDASIHDTVENLIILANNSLSSSGNVTESGCKECEELECKNIKEFLQSFVHIVQMFANTS

SEQ ID NO: 40 - protein, 114 aa, synthetic construct
  REGION 1..114: note = eIL-15-K
NWVEVISDLREIENLISSMHIDACLYTESDVSPRCSVTAMKCFLLELQVISLESGDASIHDTVENLIILANNSLSSSGNVTESGCKECEELECKNIKEFLQSFVHIVQMFANTS

SEQ ID NO: 41 - protein, 306 aa, synthetic construct
  REGION 1..306: note = IL-15Ralpha-Fc
ITCPPPMSVEHADIWVKSYSLYSRERYICNSGFKRKAGTSSLTECVLNKATNVAHWTTPSLKCIRDGGGSGGGSEPKSSDKTHTCPPCPAPELLGGPSVFLFPPKPKDILMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0176317 A1
(43) Pub. Date: Jun. 25, 2026

(54) ENGINEERED INTERLEUKIN-15 POLYPEPTIDES, COMPLEXES AND USES THEREOF

(71) Applicant: Rakuten Medical, Inc., San Diego, CA (US)

(72) Inventors: Alexey TEPLYAKOV, Middletown, MD (US); Amy THORNE, San Diego, CA (US); Barbara VISENTIN, San Diego, CA (US)

(73) Assignee: Rakuten Medical, Inc., San Diego, CA (US)

(21) Appl. No.: 19/128,915

(22) PCT Filed: Nov. 9, 2023
(86) PCT No.: PCT/US2023/079277

§ 371 Date: May 9, 2025

Related U.S. Application Data
(60) Provisional application No. 63/424,854, filed on Nov. 11, 2022.

(51) Int. Cl. C07K 14/54; A61K 38/20; A61K 40/11; A61K 40/15; A61K 40/31; A61K 41/00; A61K 45/06; A61P 35/00; C07K 14/715

(52) U.S. Cl. CPC C07K 14/5443; A61K 38/2086; A61K 40/11; A61K 40/15; A61K 40/31; A61K 41/0071; A61K 45/06; A61P 35/00; C07K 14/7155; C07K 2319/30

(57) ABSTRACT

Provided herein are engineered IL-15 (eIL-15) molecules and complexes containing engineered IL-15 molecules (eIL-15 complexes) and encoding polynucleotides. Also provided are compositions and articles of manufacture containing the conjugates, and methods for their administration to subjects to enhance immune response. In some embodiments, the eIL-15 molecules or complexes specifically bind the IL-2/15Rβγ_{c}, complex on the surface of cells. Also provided are methods and uses employing the provided eIL-15 molecules alone or in combination with other therapeutic agents or treatment regimens.

### CROSS-REFERENCE TO RELATED APPLICATIONS

This application claims priority to U.S. Provisional Application No. 63/424,854, filed Nov. 11, 2022, entitled “ENGINEERED INTERLEUKIN-15 POLYPEPTIDES, COMPLEXES AND USES THEREOF” the contents of which are incorporated by reference in their entirety.

### REFERENCE TO AN ELECTRONIC SEQUENCE LISTING

The contents of the electronic sequence listing (751702002340SEQLIST.xml; Size: 56,183 bytes; and Date of Creation: Nov. 1, 2023) is herein incorporated by reference in its entirety.

### FIELD

The present disclosure relates to engineered interleukin-15 (eIL-15) polypeptides, complexes comprising the engineered IL-15 polypeptides, conjugates, compositions, combinations, and methods and uses thereof. The disclosure is further related to nucleic acid molecules encoding the engineered IL-15 and complexes thereof described herein.

### BACKGROUND

Interleukin-15 (IL-15) is soluble protein that plays an important role in both innate and adaptive immunity. IL-15 can be used in therapeutic contexts to modulate the immune system, but challenges remain. Compositions and methods are still urgently needed to address these clinical challenges. Provided are embodiments that meet such needs.

### SUMMARY

Provided herein are engineered IL-15 (eIL-15) polypeptides. Also provided are complexes comprising the described engineered IL-15 polypeptides, nucleic acid molecules encoding the described engineered IL-15 polypeptides or complexes comprising the described engineered IL-15 polypeptides, vectors comprising the nucleic acid molecules, cells comprising the described engineered IL-15 polypeptides or complexes thereof, the nucleic acid molecules or vectors, compositions comprising the described engineered IL-15 polypeptides or complexes thereof, and methods of using any of the foregoing or the uses thereof.

Provided herein are engineered IL-15 polypeptides comprising at least 6 cysteine residues and capable of forming at least 3 intramolecular disulfide bonds. In some of any embodiments, the IL-15 polypeptide sequence is derived from a mammalian IL-15. In some of any embodiments, the IL-15 polypeptide sequence is derived from a human IL-15.

In some of any embodiments, the engineered IL-15 polypeptide comprises two amino acid substitutions in SEQ ID NO: 2, wherein the two amino acid substitutions are substituting a non-cysteine residue with a cysteine. In some of any embodiments, two of the cysteine residues are present at a position corresponding to positions 24 and 93 of SEQ ID NO:2 or corresponding to positions 29 and 102 of SEQ ID NO:2.

In some of any embodiments, the engineered IL-15 polypeptide comprises at least one amino acid substitution at a position corresponding to position 4, 10, 11, 14, 17, 18, 20, 24, 29, 32, 34, 36, 41, 52, 57, 58, 77, 80, 83, 93, 97, 102, 105, 111, or 112 of SEQ ID NO: 2. In some of any embodiments, the engineered IL-15 polypeptide comprises up to 2, 3, 4, 5, 6, 7, 8, 9, 10, 11, 12, 13, 14, 15, 16, 17, 18, 19 or 20 amino acid substitutions as compared to SEQ ID NO:2. In some of any embodiments, the engineered IL-15 polypeptide comprises one or more amino acid substitutions selected from the group consisting of N4D, N4E, K10R, K11Y, K11E, Q17S, S18N, H20N, T24C, T24L, S29C, H32N, H32S, S34K, S34R, K36S, K41L, L52R, A57P, S58P, S58Q, N77S, V80K, S83D, E93C, K97A, S102C, H105W, I111A, and N112L, with reference to positions of SEQ ID NO:2. In some of any embodiments, the engineered IL-15 polypeptide comprises 2, 3, 4, 5, 6, 7, 8, 9, 10, 11, 12, 13, 14, or 15 amino acid substitutions selected from the group consisting of N4D, N4E, K10R, K11Y, K11E, Q17S, S18N, H20N, T24C, T24L, S29C, H32N, H32S, S34K, S34R, K36S, K41L, L52R, A57P, S58P, S58Q, N77S, V80K, S83D, E93C, K97A, S102C, H105W, I111A, and N112L, with reference to positions of SEQ ID NO:2. In some of any embodiments, the engineered IL-15 polypeptide further comprises the amino acid substitution N72D, with reference to positions of SEQ ID NO:2.

In some of any embodiments, the engineered IL-15 polypeptide has at least 75% identity and less than 90% identity to SEQ ID NO:2.

In some of any embodiments, the engineered IL-15 polypeptide comprises one or more amino acid substitution in helix A, helix B, helix C, helix D of IL-15, or any combination thereof. In some of any embodiments, the engineered IL-15 polypeptide comprises one or more amino acid substitution in the loop region between helix A and helix B, between helix B and helix C, between helix C and helix D of IL-15, or any combination thereof. In some of any embodiments, the engineered IL-15 polypeptide comprises the addition of a cysteine in the loop region between helix A and helix B and/or between helix C and helix D of IL-15. In some of any embodiments, the addition of the cysteine comprises a substitution of a cysteine for another amino acid in SEQ ID NO:2.

Provided herein are complexes comprising any of the described engineered IL-15 polypeptides and a second polypeptide.

In some of any embodiments, the second polypeptide comprises an antibody or antigen-binding fragment. In some of any embodiments, the second polypeptide comprises an Fc domain or a portion thereof.

In some of any embodiments, the second polypeptide comprises a receptor molecule or domain thereof. In some of any embodiments, the second polypeptide comprises an IL-15 receptor molecule or domain thereof. In some of any embodiments, the second polypeptide comprises an IL-15 receptor sushi domain.

In some of any embodiments, the second polypeptide comprises a receptor molecule or domain thereof fused to an Fc domain or a portion thereof. In some of any embodiments, the second polypeptide comprises an IL-15 receptor molecule or domain thereof fused to an Fc domain or a portion thereof. In some of any embodiments, the second polypeptide comprises an IL-15 sushi domain fused to an Fc domain.

Provided herein are complexes comprising any of the described engineered IL-15 polypeptides and a second polypeptide comprising an IL-15 sushi domain fused to an Fc domain.

In some of any embodiments, the second polypeptide comprises the amino acid sequence set forth in SEQ ID NO:41, or an amino acid sequence that has at least 90%, 91%, 92%, 93%, 94%, 95%, 96%, 97%, 98%, or 99% sequence identity to SEQ ID NO:41.

In some of any embodiments, the engineered IL-15 polypeptide and the second polypeptide are linked non-covalently.

In some of any embodiments, the engineered IL-15 polypeptide and the second polypeptide are linked covalently.

In some of any embodiments, the complex further comprises a phthalocyanine dye.

In some of any embodiments, the phthalocyanine dye is covalently linked to the second polypeptide.

Also provided are nucleic acid molecules encoding any of the described engineered IL-15 polypeptides, or any of the described complexes.

Also provided are vectors comprising any of the described nucleic acid molecules. In some of any embodiments, the vector is an expression vector. In some of any embodiments, the vector is a mammalian vector or a viral vector.

Also provided are cells comprising any of the described engineered IL-15 polypeptides or any of the described complexes.

Also provided are cells comprising any of the described nucleic acid molecules or any of the described vectors.

In some of any embodiments, the cell is a mammalian cell.

Also provided are pharmaceutical compositions involving any of the described engineered IL-15 polypeptides, or any of the described complexes.

Also provided are method for treating a disease or condition that involve the use of any of the described engineered IL-15 polypeptides, any of the described complexes, or any of the described pharmaceutical compositions, and uses of the described engineered IL-15 polypeptides, any of the described complexes, or any of the described pharmaceutical compositions, for example, for treating a disease or condition.

Also provided are method and uses for treating a disease or condition that involve administering any of the described engineered IL-15 polypeptides in conjunction with an IL-15 receptor or functional domain thereof.

In some of any embodiments, the engineered IL-15 polypeptide is administered in conjunction with a second polypeptide involving an IL-15 receptor molecule or domain thereof fused to an Fc domain or a portion thereof. In some of any embodiments, the second polypeptide involves an IL-15 sushi domain fused to an Fc domain. In some of any embodiments, the second polypeptide involves the amino acid sequence set forth in SEQ ID NO:41, or an amino acid sequence that has at least 90%, 91%, 92%, 93%, 94%, 95%, 96%, 97%, 98%, or 99% sequence identity to SEQ ID NO:41.

Also provided are methods and uses for treating a disease or condition that involve administering any of the described engineered IL-15 polypeptides, any of the described complexes, or any of the described pharmaceutical compositions.

In some of any embodiments, the methods and uses further involve administering a second agent. In some of any embodiments, the second agent is selected from radiation, photoimmunotherapy, chemotherapy, an immune checkpoint inhibitor, a tyrosine kinase inhibitor, a CAR-T cell, or a CAR-NK cell.

Also provided are methods and uses for treating a disease or condition in a subject that involve administering any of the described engineered IL-15 polypeptides, and a photoimmunotherapy.

Also provided are methods and uses for treating a disease or condition in a subject that involve administering any of the described complexes, and a photoimmunotherapy.

Also provided are methods and uses for treating a disease or condition in a subject that involve administering any of the described pharmaceutical compositions, and a photoimmunotherapy.

In some of any embodiments, the photoimmunotherapy involves intravenously administering to the subject a targeting conjugate involving a silicon phthalocyanine dye linked to a targeting molecule, wherein the targeting molecule is capable of binding to a target on the surface of a target cell; and after administering the targeting conjugate, irradiating an area around or near a target cell at a wavelength and dose sufficient to kill the target cell, thereby treating the disease or condition.

In some of any embodiments, the engineered IL-15 polypeptide, the complex or the pharmaceutical composition is administered prior to, concurrent with or subsequent to the photoimmunotherapy.

In some of any embodiments, the target cell is a tumor cell, a cell present in the tumor microenvironment, or an immune cell. In some of any embodiments, the targeting molecule is capable of binding to Treg cells. In some of any embodiments, the targeting molecule is capable of binding to PD-L1 or PD-1. In some of any embodiments, the targeting molecule is capable of binding to EGFR.

Also provided are method and uses for modulating an immune response in a subject that involve administering any of the described engineered IL-15 polypeptides, any of the described complexes, or any of the described pharmaceutical compositions to a subject.

In some of any embodiments, modulating the immune response treats a disease or condition in the subject. In some of any embodiments, the disease or condition is selected from the group consisting of a cancer, a tumor, an infection, a viral infection, an immunocompromised state, and an immune deficiency.

In some of any embodiments, modulating the immune response increases the immune response to vaccination.

In some of any embodiments, the immune response is an increase is one or more immune modulating molecules in the treated subject as compared to prior to the treatment.

### BRIEF DESCRIPTION OF THE DRAWINGS

FIGS. 1A-1I depict dose-dependent proliferation of murine CTLL2 T cells, in response to treatment with exemplary purified engineered IL-15 (eIL-15) molecule complexes containing one or two amino acid substitutions.

FIGS. 2A-2D depict dose-dependent CTLL2 cell proliferation in response to treatment with exemplary purified eIL-15 molecules containing multiple amino acid substitutions, eIL-15-A, eIL-15-C, eIL-15-D, eIL-15-E, eIL-15-F, eIL-15-G, eIL-15-H, eIL-15-I, eIL-15-J, and eIL-15-K, in complex with soluble IL-15 receptor alpha (IL-15Rα); eIL-15 complexes containing T24C/E93C substituted eIL-15 molecules; recombinant IL-15 molecules (rIL-15); or control IL-15 complexes and corresponding EC₅₀ values.

FIGS. 3A-3C depict dose-dependent proliferation of human megakaryoblastic leukemia M-07e cells in response to treatment with exemplary eIL-15 complexes containing eIL-15-A, eIL-15-C, eIL-15-D, eIL-15-E, eIL-15-F, eIL-15-G, eIL-15-H, eIL-15-I, eIL-15-J, and eIL-15-K molecules; eIL-15 complexes containing T24C/E93C substituted eIL-15 molecules; recombinant IL-15 molecules; and/or control IL-15 complexes and corresponding EC₅₀ values.

FIGS. 4A-4C depict dose-dependent proliferation of primary human CD8-expressing T cells (huCD8⁺ T cells) in response to treatment with exemplary eIL-15 complexes containing eIL-15-A, eIL-15-B, eIL-15-C, eIL-15-D, eIL-15-E, eIL-15-F, eIL-15-G, eIL-15-H, eIL-15-I, eIL-15-J, and eIL-15-K molecules; eIL-15 complexes containing T24C/E93C substituted eIL-15 molecules; recombinant IL-15 molecules; and/or control IL-15 complexes and corresponding EC₅₀ values.

FIG. 5 depicts the dose-dependent proliferation of live primary human CD8⁺ T cells (huCD8+ T cells) in response to treatment with exemplary eIL-15 complexes containing eIL-15-A (open circle) and eIL-15-C (open square) molecules; recombinant IL-15 molecules (rIL-15); control IL-15 complexes; and recombinant IL-2 molecules (rIL-2) measured by flow cytometry and corresponding EC₅₀ values.

FIG. 6 depicts the number of cell divisions of CD8⁺ primary human T cells in response to increasing doses of eIL-15-A complex, or eIL-15-C complex, recombinant IL-2 (rIL-2), recombinant IL-15 (rIL-15), control IL-15 complex, and no treatment (NT) by indicating the % CD8+ T cells in each of generations G₀-G₈⁺.

FIG. 7A depicts the % of CD8⁺ primary human T cells expressing T cell activation marker CD25 (CD25⁺ cells) in response to treatment with increasing doses of eIL-15-A complex, eIL-15-C complex, recombinant IL-2 (rIL-2), recombinant IL-15 (rIL-15), control IL-15 complex, and no treatment (NT) and provides the corresponding EC₅₀ values for each treatment. FIG. 7B depicts the relative level of CD25 expression (MFI) of CD8⁺ primary human T cells expressing CD25 (CD25⁺ cells) in response to treatment with increasing doses of eIL-15-A complex, eIL-15-C complex, recombinant IL-2 (rIL-2), recombinant IL-15 (rIL-15), control IL-15 complex, and no treatment (NT) and provides the corresponding EC₅₀ values for each treatment.

FIG. 8A depicts the % of CD8⁺ primary human T cells expressing T cell activation marker CD69 (CD69⁺ cells) responsive to treatment with increasing doses of eIL-15-A complex, eIL-15-C complex, recombinant IL-2 (rIL-2), recombinant IL-15 (rIL-15), control IL-15 complex, and no treatment (NT) and provides the corresponding EC₅₀ values for each treatment.

FIG. 8B depicts the relative level of CD25 expression (MFI) of CD8⁺ primary human T cells expressing CD69 (CD69⁺ cells) responsive to treatment with increasing doses of eIL-15-A complex, eIL-15-C complex, recombinant IL-2 (rIL-2), recombinant IL-15 (rIL-15), control IL-15 complex, and no treatment (NT) and provides the corresponding EC₅₀ values for each treatment.

FIG. 9A depicts the % natural killer (NK) cells in response to treatment with increasing doses of eIL-15-A complex, eIL-15-C complex, recombinant IL-2 (rIL-2), control IL-15 complex (control), and no treatment (no cytokine). FIG. 9B depicts the % proliferating NK cells in response to treatment with increasing doses of eIL-15-A complex, eIL-15-C complex, recombinant IL-2 (rIL-2), control IL-15 complex (control), and no treatment (no cytokine).

FIG. 10 depicts the cytotoxic activity of NK cells against human leukemia (K-562) cells following treatment with increasing doses of eIL-15-A complex, eIL-15-C complex, recombinant IL-2 (rIL-2), and control IL-15 complex (control), with an effector (NK) cell to target (K-562) cell ratio of 5:1.

FIG. 11 depicts the ADCC activity of NK cells against Cetuximab antibody-bound epithelial squamous cell carcinoma (Cal 27) cells following treatment with increasing amount of eIL-15-C complex or recombinant IL-2 (rIL-2), with an effector to target ratio of 1:1.

FIG. 12A depicts the tumor growth in a mouse xenograft model following treatment with saline, anti-cancer photoimmunotherapy (PIT), eIL-15-A complex (eIL-15-A), or a combination of photoimmunotherapy and eIL-15-A complex (eIL-15-A+PIT). FIG. 12B depicts the tumor growth in a mouse xenograft model following treatment with saline, anti-cancer photoimmunotherapy (PIT), eIL-15-C complex (eIL-15-C), or a combination of photoimmunotherapy and eIL-15-C complex (eIL-15-C+PIT).

FIG. 13 depicts survival of a mouse xenograft model following treatment with saline, anti-cancer photoimmunotherapy (PIT), eIL-15-C complex (eIL-15-C), or a combination of photoimmunotherapy and eIL-15-C complex (eIL-15-C+PIT).

FIG. 14A depicts the percentage of T (CD3+) cells among live cells in the peripheral blood and spleen homogenates harvested from tumor-bearing mice following treatments with saline, eIL-15-A complex (eIL-15-A), or control IL-15 complex (control IL-15). FIGS. 14B and 14C depict the peripheral T (CD3+) cell counts in the blood (FIG. 14B) and in spleen homogenates (FIG. 14C) harvested from tumor-bearing mice following treatment with saline, eIL-15-A complex (eIL-15-A), or control IL-15 complex (control IL-15).

FIGS. 15A and 15B depict cytotoxic T (CD3+, CD8+) cells as a percentage of live cells (FIG. 15A) and as a percentage of T (CD3+) cells (FIG. 15B) in the peripheral blood and spleen homogenates harvested from tumor-bearing mice following treatments with saline, eIL-15-A complex (eIL-15-A), or control IL-15 complex (control IL-15). FIGS. 15C and 15D depict the peripheral cytotoxic T (CD3+, CD8+) cell counts in the blood (FIG. 15C) and in spleen homogenates (FIG. 15D) harvested from tumor-bearing mice following treatment with saline, eIL-15-A complex (eIL-15-A), or control IL-15 complex (control IL-15).

FIGS. 16A and 16B depict helper T (CD3+, CD4+) cells as a percentage of live cells (FIG. 16A) and as a percentage of T (CD3+) cells (FIG. 16B) in the peripheral blood and spleen homogenates harvested from tumor-bearing mice following treatments with saline, eIL-15-A complex (eIL-15-A), or control IL-15 complex (control IL-15). FIGS. 16C and 16D depict the peripheral helper T (CD3+, CD4+) cell counts in the blood (FIG. 16C) and in spleen homogenates (FIG. 16D) harvested from tumor-bearing mice following treatment with saline, eIL-15-A complex (eIL-15-A), or control IL-15 complex (control IL-15).

FIG. 17A depicts the percentage of NK (CD49b+, CD3−) cells among live cells in the peripheral blood and spleen homogenates harvested from tumor-bearing mice following treatments with saline, eIL-15-A complex (eIL-15-A), or control IL-15 complex (control IL-15).

FIGS. 17B and 17C depict the peripheral NK (CD3−, CD49b+) cell counts in the blood (FIG. 17B) and in spleen homogenates (FIG. 17C) harvested from tumor-bearing mice following treatment with saline, eIL-15-A complex (eIL-15-A), or control IL-15 complex (control IL-15).

FIG. 18A depicts the percentage of NK-T (CD49b+, CD3+) cells among live cells in the peripheral blood and spleen homogenates harvested from tumor-bearing mice following treatments with saline, eIL-15-A complex (eIL-15-A), or control IL-15 complex (control IL-15).

FIGS. 18B and 18C depict the peripheral NK-T (CD49b+, CD3+) cell counts in the blood (FIG. 18B) and in spleen homogenates (FIG. 18C) harvested from tumor-bearing mice following treatment with saline, eIL-15-A complex (eIL-15-A), or control IL-15 complex (control IL-15).

FIG. 19 depicts the ratio of CD4:CD8 cells in the in the peripheral blood and spleen homogenates harvested from tumor-bearing mice following treatments with saline, eIL-15-A complex (eIL-15-A), or control IL-15 complex (control IL-15).

### DETAILED DESCRIPTION

Provided herein are engineered IL-15 (eIL-15) polypeptides. Also provided are complexes comprising the described engineered IL-15 polypeptides, nucleic acid molecules encoding the described engineered IL-15 polypeptides or complexes comprising the described engineered IL-15 polypeptides, vectors comprising the nucleic acid molecules, cells comprising the described engineered IL-15 polypeptides or complexes thereof, the nucleic acid molecules or vectors, compositions comprising the described engineered IL-15 polypeptides or complexes thereof, and methods of using any of the foregoing or the uses thereof.

Interleukin-15 (IL-15) is a member of the four alpha-helix bundle family of lymphokines that plays an important role in both innate and adaptive immunity, and can be used in therapeutic applications, for example to bolster, augment, or enhance the immune response in certain contexts. However, challenges exist in using IL-15 for therapeutic uses. There exists an unmet need to provide a suitable therapeutic form of IL-15 that demonstrates greater efficacy at lower dosages when administered to an organism in need thereof for purposes of modulating or enhancing immune responses. Such a therapeutic would allow for the administration of less cytokine while simultaneously providing for the augmentation of the hosts immune system beyond the effects of IL-15 alone. Such a molecule could be used in combination with other clinical treatments that would benefit from augmented immune system activities, such as cancer treatments, treatments of viral, bacterial, or fungal infections, treatments for impaired immunity, and/or vaccine.

Provided herein are embodiments that meet such needs. In some aspects, provided are engineered IL-15 polypeptides, complexes comprising the provided engineered IL-15 polypeptides, nucleic acid molecules encoding the provided engineered IL-15 polypeptides or complexes comprising the provided engineered IL-15 polypeptides, vectors comprising the provided nucleic acid molecules, cells comprising the provided engineered IL-15 polypeptides or complexes thereof, the nucleic acid molecules or vectors, compositions comprising the provided engineered IL-15 polypeptides or complexes thereof, and methods of using any of the foregoing or the uses thereof. The provided embodiments are based on the observation that engineered IL-15 polypeptides, in some contexts when complexed with a second polypeptide, results in enhanced immune response, and in some contexts, provide improved therapeutic outcome of other therapies, such as an anti-tumor therapy such as a photoimmunotherapy (PIT).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. ENGINEERED IL-15 MOLECULES AND ENCODING POLYNUCLEOTIDES

Interleukin-15 (IL-15) is a member of the four alpha-helix bundle family of lymphokines. IL-15 is a soluble protein that plays an important role in both innate and adaptive immunity. IL-15 plays a multifunctional role in development and control of the immune system. In particular, IL-15 can influence the function, activation, development, survival, and proliferation of CD8+ T cells, NK cells, killer T cells, B cells, intestinal intraepithelial lymphocytes (IEL) and/or antigen-presenting cells (APC). IL-15 may also be involved in mediating long term memory CD8+ T cell proliferation and survival.

The cell-surface receptor for IL-15 comprises three subunits: IL-15 receptor (IL-15R) α, IL-2Rβ (also known as IL-15Rβ, CD122, and p75), and γ_{c} (also known as CD132 and p65). The ectodomain (ECD) of IL-15Rα (amino acids 1-66 of the mature IL-15-Rα sequence) consists of a single sushi domain which is necessary for IL-15 binding and IL-15Rα function (Xq et al., J Immunol. (2001); 167(1):277-282), a membrane-proximal proline-threonine-rich (PT) region, and a linker/hinge region that connects the sushi domain and the PT region. The ectodomain of IL-2/15Rβ and γ_{c} each consist of two fibronectin-type III domains, which participate in binding IL-15. The signaling pathway of IL-15 begins with binding to IL-15Rα receptor, with subsequent presentation to surrounding cells bearing IL-2/15Rβγ_{c} complex on their cell surface, whereby signaling pathways, including Jak1/Jak3 and Stat3/Stat5, Ras/mitogen-activated protein kinase, and phosphatidylinositol 3-kinase pathways, are activated.

IL-15 binds IL-15Rα with high affinity (K_{D}=30-100 pM), while binding the IL-15Rβγ_{c} signaling complex with lower affinity (K_{D}=10-30 nM). Similar to IL-15, IL-15Rα is thought to be expressed by a wide variety of cell types but not necessarily in conjunction with IL-2Rβ and IL-2Rγ.

IL-15 molecules can be used to modulate the immune system and immunogenic responses. For example, IL-15 administration can be employed to bolster immune responses or augment immune system reconstitution.

Wild-type human IL-15 is translated as a 162 amino acid (aa) prepropeptide (SEQ ID NO: 1), containing a signal sequence (aa 1-19 of SEQ ID NO: 1), a propeptide (aa 30-48 of SEQ ID NO: 1) and the IL-15 mature sequence (˜aa 44-162 of SEQ ID NO: 1; set forth in SEQ ID NO: 2). Mature IL-15 (SEQ ID NO: 2) is composed of a four-helix bundle, containing helix A (hA; ˜aa 1-16 of SEQ ID NO: 2), hB (˜aa 36-53 of SEQ ID NO: 2), hC (˜aa 57-74 of SEQ ID NO: 2), and hD (˜aa 96-111 of SEQ ID NO: 2), oriented in an up-up-down-down topology, linked together by loops that lack substantial secondary structure. The secondary structures are indicated relative to the amino acids of the mature IL-15 sequence (SEQ ID NO:2) in Table 1. It is understood that the exact starting and ending amino acids of a secondary structure may vary by one, two, or three amino acids depending on the method for determination or prediction. Two disulfide bridges, between the cysteine of position 35 (C35) with the cysteine at position 85 (C85), and the cysteine at position 42 (C42) with the cysteine at position 88 (C88), of the mature sequence (SEQ ID NO: 2), help to stabilize the conformation of the hC-hD loop (˜aa 75-95 of SEQ ID NO: 2) that engages in contacts with IL-15Rα. The cysteine residues involved in the disulfide bridges are indicated as “dsb” in Table 1. Also indicated in Table 1 are specific amino acids determined to be involved in interactions with IL-15 Rα (indicated as Rα), interactions with IL-2Rβ (indicated as Rβ), and interactions with γ_{c} (indicated as Rγ) (Chirifu et al., (2007) Nat Immunol. 8(9):1001-1007; Ring et al., (2012) Nat Immunol. 13(12):1187-11-95; Sousa et al., (2019) Molecules. 24(18):3261). The interactions indicated can be involved in direct contact with the indicated receptor subunit, can form hydrogen bonds, including water-mediated hydrogen bonds, involved in the intramolecular association with the indicated receptor, can form salt bridges with amino acids from the indicated receptor subunit, and/or can be involved in van der Waals contacts with the indicated receptor subunit. The strength of the interaction is dependent on the type of interaction/contact.

TABLE 1

| 2° structure | helix A | | | | | | | | | | | | | | | | hC-hB loop | | | |
|---|---|---|---|---|---|---|---|---|---|---|---|---|---|---|---|---|---|---|---|---|
| numbering | 1 | 2 | 3 | 4 | 5 | 6 | 7 | 8 | 9 | 10 | 11 | 12 | 13 | 14 | 15 | 16 | 17 | 18 | 19 | 20 |
| aa | N | W | V | N | V | I | S | D | L | K | K | I | E | D | L | I | Q | S | M | H |
| interaction | Rβ | | | Rβ | | | Rβ | Rβ | | Rβ | Rβ | | | | | | | | | |
| 2° structure | hA-hB loop cont. | | | | | | | | | | | | | | | helix B | | | | |
| numbering | 21 | 22 | 23 | 24 | 25 | 26 | 27 | 28 | 29 | 30 | 31 | 32 | 33 | 34 | 35 | 36 | 37 | 38 | 39 | 40 |
| aa | I | D | A | T | L | Y | T | E | S | D | V | H | P | S | C | K | V | T | A | M |
| interaction | | Rα | Rα | Rα | | Rα | | | Rγ | Rγ | Rγ | Rγ | | | dsb | | | | | |
| 2° structure | helix B cont. | | | | | | | | | | | | | hB-hC loop | | | helix C | | | |
| numbering | 41 | 42 | 43 | 44 | 45 | 46 | 47 | 48 | 49 | 50 | 51 | 52 | 53 | 54 | 55 | 56 | 57 | 58 | 59 | 60 |
| aa | K | C | F | L | L | E | L | Q | V | I | S | L | E | S | G | D | A | S | I | H |
| interaction | | dsb | | | Rα | Rα | | | Rα | | | | Rα | | | | | | | |
| 2° structure | helix C cont. | | | | | | | | | | | | | | hC-hD loop | | | | | |
| numbering | 61 | 62 | 63 | 64 | 65 | 66 | 67 | 68 | 69 | 70 | 71 | 72 | 73 | 74 | 75 | 76 | 77 | 78 | 79 | 80 |
| aa | D | T | V | E | N | L | I | I | L | A | N | N | S | L | S | S | N | G | N | V |
| interaction | Rβ | Rβ | | Rβ | Rβ | | | Rβ | Rβ | | | Rβ | | | | | | | | |

| | | |
|---|---|---|
| 2° structure | hC-hD loop | helix D |

| | | | | | | | | | | | | | | | | | | | | |
|---|---|---|---|---|---|---|---|---|---|---|---|---|---|---|---|---|---|---|---|---|
| numbering | 81 | 82 | 83 | 84 | 85 | 86 | 87 | 88 | 89 | 90 | 91 | 92 | 93 | 94 | 95 | 96 | 97 | 98 | 99 | 100 |
| aa | T | E | S | G | C | K | E | C | E | E | L | E | E | K | N | I | K | E | F | L |
| interaction | | | | | dsb | | Rα | dsb | Rα | Rα | | Rα | Rα | | | | | | | |

| 2° structure | helix D cont. | | | | | | | | | | | | | |
|---|---|---|---|---|---|---|---|---|---|---|---|---|---|---|
| numbering | 101 | 102 | 103 | 104 | 105 | 106 | 107 | 108 | 109 | 110 | 111 | 112 | 113 | 114 |
| aa | Q | S | F | V | H | I | V | Q | M | F | I | N | T | S |
| interaction | | | | | Rγ | | | Rγ | Rγ | | Rγ | Rγ | | |

Mutations in amino acids in direct or indirect contact with receptor subunits, or amino acids adjacent to those in direct or indirect contact with receptor subunits, can affect receptor binding and/or IL-15 signaling.

Provided in some aspects are engineered IL-15 (eIL-15) molecules, such as eIL-15 polypeptides. In some aspects, the eIL-15 molecules are provided in complex with all or a portion of an IL-15Rα subunit. In some aspects, the provided eIL-15 molecules exhibit increased binding to the IL-2/15Rβγ_{c} complex. In some aspects, the provided eIL-15 molecules induce increased IL-15 signaling of the IL-2/15Rβγ_{c} complex. In some aspects, the provided eIL-15 molecules induce proliferation of immune cells, such as proliferation of CD8+ T cells, NK cells, killer T (NK-T) cells, B cells, intestinal intraepithelial lymphocytes (IEL) and/or antigen-presenting cells (APC).

In some aspects, the provided eIL-15 molecules can enhance the immune response in a subject. In some aspects, the provided eIL-15 molecules increase the ratio of cytotoxic cells to immunosuppressive cells (e.g., regulatory T cells (Tregs)) in a subject, such as in the blood of a subject. In some examples, the provided eIL-15 molecules increase the ratio of CD8+ T cells to Tregs (CD8:Treg). In some examples, the provided eIL-15 molecules increase the ratio of natural killer cells to Tregs (NK:Treg). In some examples, the provided eIL-15 molecules increase the ratio of natural killer T cells to Tregs (NK-T:Treg).

In some aspects, the provided eIL-15 molecules can exhibit synergistic effects when used in combination with a treatment that is enhanced by increased immune activation. In some examples, the provided eIL-15 molecules can enhance anti-cancer treatments. In some examples, the provided eIL-15 molecules can enhance response to vaccination in a subject. In some examples, the provided eIL-15 molecules can increase the efficacy of an infection treatment, such as a treatment of a viral infection, bacterial infection, or fungal infection. In some examples, the provided eIL-15 molecules can reduce immunity impairment, as a monotherapy or in combination with other treatment(s).

A. Engineered IL-15 (eIL-15) Molecules and Complexes

Provided are engineered IL-15 (eIL-15) molecules. Also provided are eIL-15 molecules in complex with a second polypeptide comprising at least a portion of an IL-15 receptor alpha (IL-15Rα), such as the ectodomain (ECD) or sushi domain of IL-15Rα. In some embodiments, the provided eIL-15:IL-15Rα complex further comprises an antibody Fc domain, such as a human antibody Fc domain. In some embodiments, the Fc domain is linked or fused to the IL-15Rα, or portion of the IL-15Rα polypeptide, with or without a linker. In some embodiments, the linker is cleavable. In some embodiments, the linker is not cleavable.

1. Exemplary Engineered IL-15 (eIL-15) Molecules

Provided are engineered IL-15 (eIL-15) molecules. In some embodiments the eIL-15 molecules are engineered to substitute two naturally occurring amino acids for cysteine that are capable of forming a disulfide bond. In some embodiments, the provided eIL-15 molecules contain substitutions at amino acid positions that are involved in contacts with one or more subunits of the IL-15 receptor (IL-15R). In some embodiments, the provided eIL-15 molecules contain substitutions at amino acid positions that are involved in contacts with the IL-15Rα subunit. In some embodiments, the provided eIL-15 molecules contain substitutions at amino acid positions that are involved in contacts with the IL-2/15 Rβ subunit. In some embodiments, the provided eIL-15 molecules contain substitutions at amino acid positions that are involved in contacts with the γ_{c} subunit of IL-2/15R. Also, among the provided eIL-15 molecules are those having sequences at least at or about 90%, about 91%, about 92%, about 93%, about 94%, about 95%, about 96%, about 97%, about 98%, or about 99% identical to the sequences described herein.

In some embodiments, the eIL-15 molecule contains at least 2 amino acid substitutions that introduce two cysteine residues capable of forming a new disulfide bond in the eIL-15 molecule. In some embodiments, the substituted pairs included replacing the glutamic acid corresponding to position 13 and the leucine at position 100 of SEQ ID NO: 2 with cysteine (E13C/L100C; SEQ ID NO: 26), replacing the leucine corresponding to position 15 and the isoleucine at position 59 of SEQ ID NO: 2 with cysteine (L15C/I59C; SEQ ID NO: 27), replacing the threonine corresponding to position 24 and glutamic acid at position 93 of SEQ ID NO: 2 with cysteine (T24C/E93C; SEQ ID NO: 28), or replacing the serines corresponding to positions 29 and 102 of SEQ ID NO: 2 with cysteine (S29C/S102C; SEQ ID NO: 29).

In some embodiments, the eIL-15 contains the cysteine substitutions T24C/E93C. In some embodiments, the eIL-15 contains the cysteine substitutions S29C/102C.

In addition to the cysteine pair substitutions, additional amino acid substitutions were selected from the following: V3I, V3W, N4D, N4E, K10R, K11E, K11Y, D14S, D14N, Q17S, S18N, H20N, A23P, T24L, T24P, H32N, H32S, S34K, S34R, K36S, K41L, L45I, E46R, L52R, A57P, N72T, N77S, S58P, S58Q, V80K, S83D, K97A, H105E, H105W, I111A, N112L, corresponding to the positions in the amino acid sequence set forth in SEQ ID NO: 2.

In some embodiments, the eIL-15 contains a cysteine pair substitution as described and 2, 3, 4, 5, 6, 7, 8, 9, 10, 11 or 12 additional amino acid substitutions selected from the following: V3I, V3W, N4D, N4E, K10R, K11E, K11Y, D14S, D14N, Q17S, S18N, H20N, A23P, T24L, T24P, H32N, H32S, S34K, S34R, K36S, K41L, L45I, E46R, L52R, A57P, N72T, N77S, S58P, S58Q, V80K, S83D, K97A, H105E, H105W, I111A, N112L, corresponding to the positions in the amino acid sequence set forth in SEQ ID NO: 2.

In some embodiments, the eIL-15 contains 2, 3, 4, 5, 6, 7, 8, 9, 10, 11, 12, 13, 14, 15, 16, 17, 18, 19 or 20 amino acid substitutions as compared to SEQ ID NO:2. In some embodiments, the eIL-15 contains any two or more amino acid substitutions as described herein compared to the sequence set forth in SEQ ID NO:2 and exhibits at least 75%, 80%, 85%, 86%, 87%, 88%, 89%, 90%, 91%, 92%, 93%, 94%, 95%, 96% or 97% sequence identity to SEQ ID NO:2.

In some embodiments, the eIL-15 contains two or more amino acid substitutions as described herein and exhibits at least 75% sequence identity and less than 96% sequence identity to SEQ ID NO:2. In some embodiments, the eIL-15 contains two or more amino acid substitutions as described herein and exhibits at least 75% sequence identity and less than 95% sequence identity to SEQ ID NO:2. In some embodiments, the eIL-15 contains two or more amino acid substitutions as described herein and exhibits at least 75% sequence identity and less than 94% sequence identity to SEQ ID NO:2. In some embodiments, the eIL-15 contains two or more amino acid substitutions as described herein and exhibits at least 75% sequence identity and less than 93% sequence identity to SEQ ID NO:2. In some embodiments, the eIL-15 contains two or more amino acid substitutions as described herein and exhibits at least 75% sequence identity and less than 92% sequence identity to SEQ ID NO:2. In some embodiments, the eIL-15 contains two or more amino acid substitutions as described herein and exhibits at least 75% sequence identity and less than 91% sequence identity to SEQ ID NO:2. In some embodiments, the eIL-15 contains two or more amino acid substitutions as described herein and exhibits at least 75% sequence identity and less than 90% sequence identity to SEQ ID NO:2.

In some embodiments, the eIL-15 contains four or more amino acid substitutions as described herein and exhibits at least 80% and less than 96% sequence identity to SEQ ID NO:2. In some embodiments, the eIL-15 contains five or more amino acid substitutions as described herein and exhibits at least 80% and less than 95% sequence identity to SEQ ID NO:2. In some embodiments, the eIL-15 contains ten or more amino acid substitutions as described herein and exhibits at least 80% and less than 92% sequence identity to SEQ ID NO:2. In some embodiments, the eIL-15 contains twelve or more amino acid substitutions as described herein and exhibits at least 80% and less than 90% sequence identity to SEQ ID NO:2.

In some embodiments, the eIL-15 contains amino acid substitutions K10R/K11Y/D14S/Q17S/H20N/T24C/S34K/K36S/K41L/A57P/V80K/S83D/E93C. In some embodiments, the eIL-15 contains amino acid substitutions K10R/K11Y/L15C/Q17S/S18N/T24P/S34K/K36S/K41L/A57P/I59C/H105W/N112L. In some embodiments, the eIL-15 contains amino acid substitutions K10R/K11E/D14S/S18N/T24L/S29C/S34K/S58P/V80K/S83D/S102C/H105W/N112L. In some embodiments, the eIL-15 contains amino acid substitutions K10R/K11E/D14S/Q17S/H20N/T24C/S34K/K36S/V80K/S83D/E93C/K97A. In some embodiments, the eIL-15 contains amino acid substitutions K10R/K11E/D14S/Q17S/H20N/T24C/S34K/K36S/V80K/S83D/E93C/H105W. In some embodiments, the eIL-15 contains amino acid substitutions K10R/K11E/D14S/Q17S/T24C/S34K/K36S/V80K/S83D/E93C/K97A/H105W. In some embodiments, the eIL-15 contains amino acid substitutions K10R/K11E/D14N/Q17S/H20N/T24C/S58Q/V80Y/S83D/E93C/K97A/H105W. In some embodiments, the eIL-15 contains amino acid substitutions K10R/K11E/D14N/H20N/T24C/S34K/K36S/V80K/S83D/E93C/K97A/H105W. In some embodiments, the eIL-15 contains amino acid substitutions K10R/K11Y/D14S/Q17S/H20N/T24C/L52R/V80K/S83D/E93C/K97A/N112L. In some embodiments, the eIL-15 contains amino acid substitutions N4D/K10R/K11E/D14N/Q17S/T24C/H32N/S34R/K36S/N77S/E93C/I111A. In some embodiments, the eIL-15 contains amino acid substitutions N4E/K10R/K11E/D14N/Q17S/T24C/H32S/S34R/K36S/N77S/E93C/I111A.

In some embodiments, the eIL-15 molecule has the amino acid sequence selected from any one of SEQ ID NOs:26-40 or an amino acid sequence that has at least 90%, 91%, 92%, 93%, 94%, 95%, 96%, 97%, 98%, or 99% sequence identity to the amino acid selected from any one of SEQ ID NOs: 26-40. In some embodiments, the eIL-15 molecules has the sequence set forth in SEQ ID NO:30. In some embodiments, the eIL-15 molecules has the sequence set forth in SEQ ID NO:31. In some embodiments, the eIL-15 molecules has the sequence set forth in SEQ ID NO:32. In some embodiments, the eIL-15 molecules has the sequence set forth in SEQ ID NO:33. In some embodiments, the eIL-15 molecules has the sequence set forth in SEQ ID NO:34. In some embodiments, the eIL-15 molecules has the sequence set forth in SEQ ID NO:35. In some embodiments, the eIL-15 molecules has the sequence set forth in SEQ ID NO:36. In some embodiments, the eIL-15 molecules has the sequence set forth in SEQ ID NO:37. In some embodiments, the eIL-15 molecules has the sequence set forth in SEQ ID NO:38. In some embodiments, the eIL-15 molecules has the sequence set forth in SEQ ID NO:39. In some embodiments, the eIL-15 molecules has the sequence set forth in SEQ ID NO:40.

Also provided are eIL-15 complexes that include any of the eIL-15 molecules provided herein. Such eIL-15 complexes include at least one eIL-15 molecule in complex with at least a portion of an IL-15 receptor alpha (IL-15Rα) subunit, such as the ectodomain (ECD) or sushi domain of IL-15Rα. In some embodiments, the provided eIL-15Rα complex further comprises an antibody Fc domain, such as a human antibody Fc domain. In some embodiments, the Fc domain is attached to the IL-15Rα subunit, or portion of the IL-15Rα subunit, with or without a linker. In some embodiments, the linker is cleavable. In some embodiments, the linker is not cleavable. In some embodiments, the IL-15Rα-Fc molecule, to be used in the eIL-15 complex with any of the eIL-15 molecules provided herein, has the amino acid sequence set forth in SEQ ID NO: 41 or a functionally equivalent amino acid sequence.

Also provided are nucleic acids, e.g., polynucleotides, encoding the antibodies and/or portions, e.g., chains, thereof. Among the provided nucleic acids are those encoding any of the eIL-15 molecules described herein. The nucleic acids may include those encompassing natural and/or non-naturally occurring nucleotides and bases, e.g., including those with backbone modifications. The terms “nucleic acid molecule”, “nucleic acid” and “polynucleotide” may be used interchangeably and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

Also provided are vectors containing the nucleic acids, e.g., polynucleotides, and host cells containing the vectors, e.g., for expressing and/or producing the eIL-15 molecules or eIL-15 complexes described herein. Also provided are methods for producing the eIL-15 molecules and eIL-15 complexes. The nucleic acid may encode an amino acid sequence comprising the eIL-15 molecule and, optionally, an IL-15Rα subunit or portion thereof, such as the ECD or sushi domain. In some embodiments, the nucleic acid, e.g., polynucleotide, encodes one or more eIL-15 molecule and one or more IL-15 Rα subunit, in any order or orientation. In some embodiments, the nucleic acid, e.g., polynucleotide, encodes an eIL-15 molecule and an IL-15 Rα subunit, and the coding sequence for the eIL-15 molecule is upstream of the coding sequence for the IL-15 Rα subunit. In some embodiments, the nucleic acid, e.g., polynucleotide, encodes an eIL-15 molecule and an IL-15 Rα subunit, and the coding sequence for the IL-15 Ra subunit is upstream of the coding sequence for the eIL-15 molecule. In some embodiments, the nucleic acid, e.g., polynucleotide, encodes the eIL-15 molecule and an IL-15 Rα subunit, and the coding sequence for the IL-15 Rα subunit is downstream of the coding sequence for the eIL-15 molecule.

In a further embodiment, one or more vectors (e.g., expression vectors) comprising such nucleic acids are provided. In a further embodiment, a host cell comprising such nucleic acids is provided. In some embodiments, a host cell comprises (e.g., has been transformed with) (1) a vector comprising a nucleic acid that encodes an amino acid sequence comprising an eIL-15 molecule and an amino acid sequence comprising the IL-15Rα subunit (e.g. IL-15Rα ECD) or (2) a first vector comprising a nucleic acid that encodes an amino acid sequence comprising an eIL-15 molecule and a second vector comprising a nucleic acid that encodes an amino acid sequence comprising the IL-15Rα subunit (e.g. IL-15Rα ECD). In some embodiments, a composition containing one or more such host cells are provided.

Also provided are methods of making the eIL-15 molecules. For recombinant production of the eIL-15 molecule, a nucleic acid sequence or a polynucleotide encoding an eIL-15 molecule, e.g., as described above, may be isolated and inserted into one or more vectors for further cloning and/or expression in a host cell. In some embodiments, the eIL-15 molecule is co expressed with the ECD or sushi domain of the IL-15Rα. Such nucleic acid sequences may be readily isolated and sequenced using conventional procedures (e.g., by using oligonucleotide probes that are capable of binding specifically to genes encoding the heavy and light chains of the antibody). In some embodiments, a method for making the eIL-15 molecule is provided, wherein the method comprises culturing a host cell comprising a nucleic acid sequence encoding the eIL-15 molecule, as provided above, a nucleic acid sequence encoding IL-15α ECD under conditions suitable for expression of the eIL-15-IL-15Rα complex, and optionally recovering the eIL-15-IL-15Rα complex from the host cell (or host cell culture medium).

In addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; HEK cells, including HEK293 cells, such as 293-6E cells; CHO cells, including CHO—S, CHO-DG44, Lec13 CHO cells, and FUT8 CHO cells; PER.C6® cells; and NSO cells. In some embodiments, the eIL-15 molecules or eIL-15-IL-15Rα complexes (e.g., eIL-15 and/or IL-15Rα ECD) may be expressed in yeast. In some embodiments, a particular eukaryotic host cell is selected based on its ability to make desired post-translational modifications to the eIL-15 molecules or eIL-15-IL-15Rα complexes.

In some embodiments, the antibody or antigen-binding fragment provided herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

The provided embodiments further include vectors and host cells and other expression systems for expressing and producing eIL-15 molecules and eIL-15 complexes, including eukaryotic and prokaryotic host cells, including bacteria, filamentous fungi, and yeast, as well as mammalian cells such as human cells, as well as cell-free expression systems.

2. Exemplary Features of eIL-15 Molecules

In some aspects, the provided eIL-15 molecules and eIL-15 complexes have one or more specified functional features, such as binding properties to the IL-2Rβ/γ_{c} (e.g., hIL-2Rβ:IL-2Rγ dimers) or stimulating cell proliferation.

### a) Binding Affinity

In some aspects, the provided eIL-15 molecules and eIL-15 complexes have one or more specified functional features, such as binding properties to the IL-2Rβ/γ_{c} (e.g., hIL-2Rβ:IL-2Rγ dimers). In some embodiments, the eIL-15 molecules or eIL-15 complexes bind to IL-2Rβ/γ_{c} with similar affinity as wild-type IL-15 or a complex containing wild-type IL-15. In some embodiments, the eIL-15 molecules or eIL-15 complexes bind to IL-2Rβ/γ_{c} with reduced affinity as compared to wild-type IL-15 or a complex containing wild-type IL-15. In some embodiments, the eIL-15 molecules or eIL-15 complexes bind to IL-2Rβ/γ_{c} with increased affinity as compared to wild-type IL-15 or a complex containing wild-type IL-15.

In some embodiments provided herein, IL-2Rβ/γ_{c} refers to a heterodimer of human IL-2Rβ subunit and human IL-2Rβ/γ_{c}, a non-human primate (e.g., cynomolgus monkey) IL-2Rβ/γ_{c} heterodimer, or a mouse IL-2Rβ/γc. In some embodiments provided herein, IL-2Rβ/γc refers to human IL-2Rβ/γ_{c} or a non-human primate (e.g., cynomolgus monkey) IL-2Rβ/γc. In some embodiments of any of the embodiments herein, IL-2Rβ/γ_{c} refers to human IL-2Rβ/γc. The observation that an eIL-15 molecule or eIL-15 complex binds to IL-2Rβ/γ_{c} does not necessarily mean that it binds to an IL-2Rβ/γ_{c} of every species. For example, in some embodiments, features of binding to IL-2Rβ/γ_{c}, such as the ability to specifically bind thereto and/or to bind with a particular affinity to a particular degree, in some embodiments, refers to the ability with respect to a human IL-2Rβ/γ_{c} heterodimer and the eIL-15 molecule or complex may not have this feature with respect to an IL-2Rβ/γ_{c} heterodimer of another species, such as mouse. In some embodiments, the eIL-15 molecule or complex binds to a mammalian IL-2Rβ/γ_{c} heterodimer, including to naturally occurring variants of IL-2Rβ and/or IL-2Rγ_{c} within the heterodimer, such as allelic variants.

Binding affinity of the eIL-15 molecule or complex to IL-2Rβ/γ_{c} can be measured by any known method, such as by a radioimmunoassay (RIA), enzyme-linked immunosorbent assay (ELISA), or surface plasmon resonance (SPR). In some embodiments, the provided eIL-15 molecules or complexes are capable of binding IL-2Rβ/γ_{c}, such as human IL-2Rβ/γ_{c}, with at least a certain affinity, as measured by any of a number of known methods.

In some embodiments, the affinity is represented by an equilibrium dissociation constant (K_{D}); in some embodiments, the affinity is represented by EC₅₀. It is within the level of a skilled artisan to determine the binding affinity of an eIL-15 molecule or complex for one or more of its cognate receptor subunits, such as by using any number of binding assays that are well known in the art.

For example, in some embodiments, a BIAcore® instrument can be used to determine the binding kinetics and constants of a complex between two proteins (e.g., an eIL-15 molecule or complex and one or more subunits of the IL-2/15R or a fragment thereof, such as an extracellular domain), using surface plasmon resonance (SPR) analysis. SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip.

Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, and other methods for detection of protein binding.

In some embodiments, the dissociation constant (K_{D}) of the eIL-15 molecule or complex to IL-2Rβ/γ_{c}, such as human IL-2Rβ/γ_{c}, is from or from about 1×10⁻¹¹ to about 1×10⁻⁹ M at 25° C.

### b) Immune Cell Proliferation and Activation

In some embodiments the eIL-15 molecule or complex stimulates the proliferation of cells, such as immune cells. In some embodiments the eIL-15 molecule or complex stimulates the proliferation of cytotoxic or cytolytic immune cells. In some embodiments, the eIL-15 molecule or complex stimulates the proliferation of T cells, such as effector T cells. In some embodiments, the eIL-15 molecule or complex stimulates the proliferation of CD8+ T cells. In some embodiments, the eIL-15 molecule or complex stimulates the proliferation of natural killer (NK) cells and/or natural killer T (NK-T) cells. In any of the embodiments, the eIL-15 molecule or complex stimulates more proliferation of cells, such as more proliferation of immune cells, when compared to the proliferation stimulated by recombinant IL-15, such as recombinant human IL-15. In any of the embodiments, the eIL-15 molecule or complex stimulates more proliferation of cells, such as more proliferation of immune cells (e.g., CD8+ T cells or NK cells), when compared to molecules or complexes containing wild-type IL-15, such as wild-type human IL-15. In some of any of the embodiments, the eIL-15 molecule or complex stimulates more proliferation of cells, such as immune cells (e.g., CD8+ T cells or NK cells), than a control IL-15 molecule with the amino acid sequence set forth in SEQ ID NO: 3 or a complex containing a control IL-15 molecule having the amino acid sequence set forth in SEQ ID NO: 3.

Cell proliferation can be measured by a variety of methods, including metabolic activity assays, cell proliferation marker assays, ATP concentration assays, DNA synthesis assays, flow cytometric assays, and cell counting assays. Such methods are known to the skilled artisan or include, but are not limited to, those described in, for example, Adnan et al., Current Pharmaceutical Biotechnology, (2016) 17(14): 1213-1221(9).

In some embodiments, the eIL-15 molecule or complex promotes the survival of cells, such as immune cells. In some embodiments the eIL-15 molecule or complex promotes the survival of cytotoxic or cytolytic immune cells. In some embodiments, the eIL-15 molecule or complex promotes the survival of T cells, such as effector T cells. In some embodiments, the eIL-15 molecule or complex promotes the survival of CD8+ T cells. In some embodiments, the eIL-15 molecule or complex promotes the survival of natural killer (NK) cells. In some embodiments, the eIL-15 molecule or complex promotes the survival of natural killer T (NK-T) cells.

In some embodiments, the eIL-15 molecule or complex enhances the activity of immune cells, such as cytotoxic immune cells (e.g., cytotoxic CD8+ T cells, natural killer (NK) cells, and natural killer T (NK-T) cells).

In some embodiments, the eIL-15 molecule or complex suppresses or inhibits the effects of immunosuppressant cells, such as regulatory T cells (Tregs). In some aspects, the eIL-15 enhances or augments the immune response by suppressing or inhibiting the effects of immunosuppressant cells, such as Tregs, thereby enhancing the effect of immune cells that would be typically suppressed by the immunosuppressant cells.

### B. Variants

In certain embodiments, the eIL-15 molecules or complexes include one or more amino acid variations, e.g., substitutions, deletions, insertions, and/or mutations, compared to the sequence of the eIL-15 molecule or complex described herein. Exemplary variants include those designed to improve the binding affinity and/or other biological properties of the eIL-15 molecule to one or more subunits of its cognate receptor. Amino acid sequence variants of an eIL-15 molecule may be prepared by introducing appropriate modifications into the nucleotide sequence encoding the eIL-15 molecule, or by peptide synthesis. Such modifications include, for example, deletions from, and/or insertions into and/or substitutions of residues within the amino acid sequences of the eIL-15 molecule. Any combination of deletion, insertion, and substitution can be made to arrive at the final construct, provided that the final construct possesses the desired characteristics, e.g., receptor binding or signal transduction.

In some embodiments, one or more residues within the eIL-15 molecule is/are substituted. In some embodiments, the substitution is made to revert a sequence or position in the sequence to a germline sequence, such as an eIL-15 molecule sequence found in the germline (e.g., human germline), for example, to reduce the likelihood of immunogenicity, e.g., upon administration to a human subject.

In certain embodiments, substitutions, insertions, or deletions may occur within the eIL-15 molecule so long as such alterations do not substantially reduce the ability of the antibody to bind the one or more subunits of the IL-2/15R or the signaling transduced by the IL-2 receptor. For example, conservative alterations (e.g., conservative substitutions as provided herein) that do not substantially reduce binding affinity may be made in eIL-15 molecules. Such alterations may, for example, be outside of receptor subunit contacting residues as described herein. In certain embodiments of the eIL-15 sequences provided above, the sequence either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the eIL-15 molecule include the fusion to the N- or C-terminus of the eIL-15 molecule or complex to an enzyme or a polypeptide which increases the serum half-life of the eIL-15 molecule or complex.

In some embodiments, the provided complexes comprising the eIL-15 molecules also comprises a phthalocyanine dye. In some of any embodiments, the phthalocyanine dye is covalently linked to the second polypeptide, for example, a second polypeptide comprising at least a portion of an IL-15 receptor alpha (IL-15Rα), such as the ectodomain (ECD) or sushi domain of IL-15Rα, and an Fc domain or a portion thereof. In some aspects, the phthalocyanine dye comprises a phthalocyanine dye, such as a silicon-phthalocyanine dye as described in, for example, WO 2017/031363, WO 2017/031367, WO 2021/207691, and WO 2022/182483. In some aspects, the phthalocyanine dye comprises IR700.

### II. METHODS OF TREATMENT AND USES OF EIL-15 MOLECULES

Provided herein methods and uses of the provided engineered IL-15 polypeptides, nucleic acid molecules encoding the provided engineered IL-15 polypeptides or complexes comprising the provided engineered IL-15 polypeptides, vectors comprising the provided nucleic acid molecules, cells comprising the provided engineered IL-15 polypeptides or complexes thereof, the nucleic acid molecules or vectors, compositions comprising the provided engineered IL-15 polypeptides or complexes thereof. In some aspects, the methods and uses relate to therapeutic uses.

Also provided herein are compositions, such as pharmaceutical compositions, containing the eIL-15 molecule or complex, and uses of such compositions, such as therapeutic uses and/or uses as a medicament. In some aspects, the compositions comprise the eIL-15 molecule or complex and a pharmaceutically acceptable carrier. In some embodiments, the composition containing the eIL-15 molecule or complex is for use in treatment or therapy, in accordance with any of the provided embodiments, such as for administration to a subject having a disease or condition, for the treatment of the disease or condition. The dosages of the eIL-15 molecule or complex to be administered to a subject are not subject to absolute limits but will depend on the nature of the composition and its active ingredients and its unwanted side effects, such as immune response against the agent, the subject being treated, and the type of condition being treated and the manner of administration. Generally, the dose will be a therapeutically effective amount, such as an amount sufficient to achieve a desired biological effect, for example an amount that is effective to decrease the size, such as volume and/or weight, of the tumor, or attenuate further growth of a tumor, or decrease undesired symptoms of a tumor.

The effects of IL-15 on the immune system makes IL-15 a useful molecule for manipulating the immune system and immunogenic responses. For example, IL-15 administration can be employed to bolster immune responses or augment immune system reconstitution. The eIL-15 molecules and complexes provided herein can be used according to methods of using wild-type IL-15 molecules and complexes for similar purposes. Dosing can be empirically determined as necessary to reduce any unwanted side effects.

In some embodiments of the provided methods and uses, the eIL-15 molecules or complexes provided herein can be administered as an adjuvant during cancer treatment, vaccination or infection to augment CD8+ T cell immunity. In some embodiments of the provided methods and uses, the eIL-15 molecules or complexes can be administered to protect a subject bacterial infection. In some embodiments of the provided methods and uses, eIL-15 molecules or complexes can be used to stimulate immunity against viruses, including viruses that suppress the immune system, such as HIV. In some embodiments of the provided methods and uses, the eIL-15 molecules can be used increase the survival of CD4+ and CD8+ lymphocytes in subjects, such as subjects with cancer or subjects infected with bacteria or virus, such as HIV. In some embodiments of the provided methods and uses, the eIL-15 molecules or complexes can be used to accelerate immune reconstitution after bone marrow transplant.

The provided eIL-15 molecules and complexes can be used in applications where augmentation of the immune response is desirable. These include increasing the efficacy of vaccines against tumors and infections as well as augmenting the ability of the body to remove cancers. In addition, eIL-15 molecules or complexes may be used in methods and uses to aid in regenerating the immune system following bone marrow transplant or in AIDS.

In some aspects of the provided methods and uses and uses can enhance, activate, induce, recruit, or support infiltration of lymphocytes into a tumor or lesion of a subject. In some embodiments, the provided methods and uses and uses activate the intratumoral innate response, resulting increased activation of intratumoral dendritic cells (e.g., activated dendritic cells). In some aspects, the provided methods and uses and uses activate the adaptive immune response, resulting in increased infiltration, proliferation, and/or activation of CD8+ T cells. In some embodiments, the provided methods and uses and uses lead to increased intratumoral infiltration of newly primed CD8+ T cells.

### A. Methods for Stimulating or Enhancing Anti-Cancer Immune Responses

In some aspects, the provided methods and uses employing compositions including an eIL-15 molecule or complex can result in an enhancement of an immune response, such as systemic and/or local immune response in the subject, which in turn can result in an enhanced response to the therapy or treatment for a tumor, a lesion or a cancer. In some aspects, the provided embodiments can stimulate, enhance, activate, induce, provoke, boost, augment, or support an immune response, such as a systemic immune response, in a subject having a tumor, a lesion or a cancer. In some embodiments, the provided method and uses results in enhancing a systemic immune response in a subject having a tumor, a lesion or a cancer. “Systemic immune response” refers to the ability of a subject's immune system to respond to an immunologic challenge or immunologic challenges, including those associated with a tumor, a lesion or a cancer, in a systemic manner. Systemic immune response can include systemic response of the subject's adaptive immune system and/or innate immune system. Systemic immune response can include anti-tumor or anti-cancer response from the subject's adaptive immune system and/or innate immune system. In some aspects, systemic immune response includes an immune response across different tissues, including the blood stream, lymph node, bone marrow, spleen and/or the tumor microenvironment, and in some cases, includes a coordinated response among the tissues and organs and various cells and factors of the tissues and organs. In some embodiments, the provided embodiments can stimulate, enhance, activate, induce, provoke, boost, augment, or support the anti-cancer or anti-tumor immune response of the subject's own immune system, including the adaptive immune system and/or innate immune system. In some aspects, the provided methods and uses can result in enhancement of an innate immune response in the subject.

In some aspects, the provided embodiments can effect tumor immunity. In such aspects, the provided embodiments prevent or impede growth of a new tumor or a metastasis. In some embodiments, inhibition of tumor growth effected by the provided embodiments leads to a durable anti-tumor response. In some embodiments, inhibition of tumor growth effected by the provided embodiments leads to prolonged progression-free survival. In some embodiments, inhibition of tumor growth effected by the provided embodiments leads to a reduced chance of relapse and/or a reduced chance of metastasis. In some aspects, the provided embodiments can effect immunity for the same tumor type or a different tumor type in the treated subject. In some aspects, the provided embodiments can inhibit growth of tumors from a different tumor lineage, i.e., a different type of tumor that arises or could arise in a treated subject.

In some aspects, the provided embodiments can stimulate or enhance a systemic response, such as a systemic immune response, against one or more primary tumors or lesions and/or one or more second tumors or lesions, such as metastatic tumors or lesions.

In some aspects, inhibition of the growth of the tumor or the lesion is dependent on the presence of CD8+ T cells. In some embodiments, prior to the administering, the subject has a tumor or a lesion having a low number or level of CD8+ T cell infiltration. In some embodiments, the number, level or activity of immune cells is increased in the tumor or in the tumor microenvironment after administering the eIL-15 molecule or complex. In some embodiments, the number or level of CD8+ T cell infiltration in the tumor or the lesion is increased after administering the eIL-15 molecule or complex.

In some aspects, the stimulated or enhanced systemic immune response includes an increase in the number and/or activity of systemic CD8⁺ T effector cells, an increase in systemic T cell cytotoxicity against tumor cells as measured using a CTL assay using cells from the spleen, the peripheral blood, the bone marrow, or the lymph nodes, an increase in the number, activity and/or priming of intratumoral CD8⁺ T effector cells in the primary or secondary (e.g., metastatic or new) tumors or lesions, an increase in systemic CD8⁺ T cell activation, an increase in systemic dendritic cell activation, an increase in dendritic cell activation in the primary or secondary (e.g., metastatic or new) tumors or lesions, an increase in intratumoral dendritic cell infiltration in the primary or secondary (e.g., metastatic or new) tumors or lesions, an increase in new T cell priming in the primary or secondary (e.g., metastatic or new) tumors or lesions, an increase in T cell diversity in the primary or secondary (e.g., metastatic or new) tumors or lesions, a decrease in systemic regulatory T cells, a decrease in regulatory T cells in the primary or secondary (e.g., metastatic or new) tumors or lesions, a decrease in systemic myeloid derived suppressor cells, a decrease in intratumoral myeloid derived suppressor cells in the primary or secondary (e.g., metastatic or new) tumors or lesions, a decrease in tumor associated fibroblasts or cancer associated fibroblasts (CAFs), in the primary or secondary (e.g., metastatic or new) tumors or lesions, or any combination thereof in the subject. In some instances, a systemic response can be assessed by sampling blood, tissue, cells or other fluid from a subject and assessing an increase in pro-inflammatory cytokines, an increase or appearance of immune cell activation markers and/or T cell diversity. In some aspects, a systemic response may be assessed by assaying cells affected directly or indirectly by the methods. For example, cell can be collected from the subject between day 4 and day 28 after treatment or any time after treatment.

In some aspects, the provided embodiments can stimulate, enhance, boost, augment, or support an immune response, such as a local response, such as a local immune response, in a subject having a tumor, a lesion or a cancer. In some embodiments, the provided method and uses results in enhancing a local response in a subject having a tumor, a lesion or a cancer. “Local immune response” refers to the immune response in a tissue or an organ to an immunologic challenge or immunologic challenges including those associated with a tumor, a lesion or a cancer. A local immune response can include the adaptive immune system and/or innate immune system. In some aspects, local immunity includes immune response concurrently occurring at different tissues, such as the blood stream, lymph node, bone marrow, spleen and/or the tumor microenvironment.

In some aspects, the stimulated or enhanced local immune response includes an increase in the number and/or activity of intratumoral CD8⁺ T effector cells (e.g., CD3⁺ CD8⁺ cells), an increase in CD8⁺ T effector cell activation, an increase in intratumoral dendritic (CD11c⁺) cell infiltration, an increase in intratumoral dendritic cell activation (e.g., CD11c⁺ CD80⁺ and/or CD11c⁺ CD40⁺), an increase in intratumoral antigen-presenting dendritic cells (CD11b⁺ CD103⁺ CD11c⁺), an increase in intratumoral new T cell priming (e.g., CD3⁺ CD8⁺ PD1⁻ cells), an increase in intratumoral T cell diversity, an increase in intratumoral neutrophils (CD11b⁺ Cy6C^{-/low} Ly6G⁺ cells), a decrease in intratumoral macrophages (e.g., CD11b⁺ F4/80⁺ cells), a decrease in intratumoral regulatory T cells (Tregs), a decrease in intratumoral myeloid derived suppressor cells (MDSCs; e.g., CD11b⁺ Ly6C⁺ Ly6G⁻ cells), a decrease in intratumoral tumor associated fibroblasts or cancer associated fibroblasts (CAFs), a decrease in the number and/or activity of intratumoral exhausted T cells, such as exhausted CD8+ T cells (e.g., PD-1⁺CTLA-4⁺CD3⁺CD8⁺ cells), or any combination thereof in the subject. In some aspects, the stimulated or enhanced local immune response is effected by any of the provided embodiments. In some aspects, the cell surface phenotype of cells, such as immune cells indicative of local immune response or innate immune response, is assessed by staining with reagents, such as labelled antibodies, that can be used to detect the expression of the marker(s) on the surface. In some aspects, the cell surface phenotype of cells, such as immune cells indicative of local immune response or innate immune response, is detected using flow cytometry.

In some cases, a local response, such as a local immune response, can be assessed by taking a blood, tissue or other sample from a subject and assessing for an increase in an anti-immune cell type in the tumor or TME and/or assessing for an increase or appearance of local immune activation markers. In some aspects, a local response, such as a local immune response, may be assessed by assaying cells affected directly or indirectly by the methods. For example, cell can be collected from the subject between day 4 and day 28 after treatment or any time after treatment.

In some aspects, the methods and uses also involve administering an additional therapeutic agent, such as an immunomodulatory agent, e.g., an immune checkpoint inhibitor. The immunomodulatory agent can be administered prior to, concurrent with or subsequent to the administration of the eIL-15 molecule or complex. In some aspects, administration of the additional therapeutic agent, such as an immunomodulatory agent, can also contribute to stimulating, enhancing, activating, inducing, augmenting or supporting an immune response, such as the subject's systemic and/or local immune response, including anti-cancer or anti-tumor responses. Exemplary additional therapeutic agents, compositions, combinations, methods and uses include those described herein, e.g., in Section IV.

### III. METHODS OF ADMINISTRATION AND FORMULATIONS

Also provided are compositions including the eIL-15 molecules and complexes including pharmaceutical compositions and formulations.

In some embodiments, the eIL-15 molecule or complex may be administered either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the eIL-15 molecule or complex is administered intravenously. In some embodiments, the eIL-15 molecule or complex is administered parenterally. In some embodiments, the eIL-15 molecule or complex is administered enterally. In some embodiments, the eIL-15 molecule or complex is administered by local injection. In some embodiments, the eIL-15 molecule or complex is administered as a topical application.

The compositions comprising the eIL-15 molecule or complex can be administered locally or systemically using any method known in the art, for example to subjects having a disease or condition benefitting from IL-15 administration. In some embodiments, the eIL-15 molecule or complex can be administered to a subject who has a tumor, such as a cancer, or who has had a tumor previously removed, for example via surgery. Although specific examples are provided, one skilled in the art will appreciate that alternative methods of administration of the disclosed agents can be used. Such methods may include for example, the use of catheters or implantable pumps to provide continuous infusion over a period of several hours to several days into the subject in need of treatment.

In some embodiments, the eIL-15 molecule or complex is administered by parenteral means, including direct injection or infusion into a tumor, such as intratumorally. In some embodiments, the eIL-15 molecule or complex is administered to the tumor by applying the agent to the tumor, for example by bathing the tumor in a solution containing the eIL-15 molecule or complex, or by pouring the agent onto the tumor. In addition, or alternatively, the eIL-15 molecule or complex can be administered systemically, for example intravenously, intramuscularly, subcutaneously, intradermally, intraperitoneally, subcutaneously, or orally, to a subject having a tumor, such as cancer.

In some embodiments, the compositions used for administration of the eIL-15 molecule or complex contain an effective amount of the agent along with conventional pharmaceutical carriers and excipients appropriate for the type of administration contemplated. For example, in some embodiments, parenteral formulations may contain a sterile aqueous solution or suspension of the eIL-15 molecule or complex. In some embodiments, compositions for enteral administration may contain an effective amount of the eIL-15 molecule or complex in aqueous solution or suspension that may optionally include buffers, surfactants, thixotropic agents, and flavoring agents.

In some embodiments, the eIL-15 is formulated in a pharmaceutically acceptable buffer, such as that containing a pharmaceutically acceptable carrier or vehicle. Generally, the pharmaceutically acceptable carriers or vehicles, such as those present in the pharmaceutically acceptable buffer, can be any known in the art. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds.

Pharmaceutically acceptable compositions generally are prepared in view of approvals for a regulatory agency or other agency prepared in accordance with generally recognized pharmacopeia for use in animals and in humans. In some embodiments, the eIL-15 molecule or complex is formulated together with an additional therapeutic agent.

Pharmaceutical compositions can include carriers such as a diluent, adjuvant, excipient, or vehicle with which the compound is administered. Examples of suitable pharmaceutical carriers are described in “Remington's Pharmaceutical Sciences” by E. W. Martin. Such compositions will contain a therapeutically effective amount of the active compound (e.g., eIL-15 molecule or complex and/or one or more additional therapeutic agent(s)), generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidone, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

In some embodiments, pharmaceutical preparation can be in liquid form, for example, solutions, syrups or suspensions. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). In some cases, pharmaceutical preparations can be presented in lyophilized form for reconstitution with water or other suitable vehicle before use.

In some embodiments, the nature of the pharmaceutically acceptable buffer, or carrier, depends on the particular mode of administration being employed. For instance, in some embodiments, parenteral formulations may comprise injectable fluids that include pharmaceutically and physiologically acceptable fluids such as water, physiological saline, balanced salt solutions, aqueous dextrose, or glycerol as a vehicle. In some embodiments, for solid compositions, for example powder, pill, tablet, or capsule forms, non-toxic solid carriers can include, for example, pharmaceutical grades of mannitol, lactose, starch, or magnesium stearate. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In addition to biologically neutral carriers, pharmaceutical compositions to be administered can in some embodiments contain minor amounts of non-toxic auxiliary substances, such as wetting or emulsifying agents, preservatives, and pH buffering agents, for example sodium acetate or sorbitan monolaurate.

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the eIL-15 molecules or complexes described herein can include lyophilized formulations and aqueous solutions.

The formulation or composition may also contain more than one active ingredient useful for the particular indication, disease, or condition being treated with the eIL-15 molecule or complex, preferably those with activities complementary to the eIL-15 molecule or complex, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The compounds can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the eIL-15 molecule or complex in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Compositions can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, optic, inhalational, buccal (e.g., sublingual), and transdermal administration or any route. Other modes of administration also are contemplated. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant.

Parenteral administration, generally characterized by injection, either subcutaneously, intramuscularly, intratumorally, intravenously or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain an activator in the form of a solvent such as pH buffering agents, metal ion salts, or other such buffers. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) also is contemplated herein. The percentage of active compound contained in such parenteral compositions is highly dependent on the specific nature thereof, as well as the activity of the compound and the needs of the subject.

Injectables are designed for local and systemic administration. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or non-aqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, non-aqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Non-aqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate.

If administered intravenously, suitable carriers include physiological saline or phosphate buffered saline (PBS), and solutions containing thickening and solubilizing agents, such as glucose, polyethylene glycol, and polypropylene glycol and mixtures thereof.

The composition can be formulated for single dosage administration or for multiple dosage administration. The agents can be formulated for direct administration. The composition can be provided as a liquid or lyophilized formulation. Where the composition is provided in lyophilized form it can be reconstituted just prior to use by an appropriate buffer, for example, a sterile saline solution.

Compositions also can be administered with other biologically active agents, either sequentially, intermittently or in the same composition. Administration also can include controlled release systems including controlled release formulations and device-controlled release, such as by means of a pump.

The most suitable route in any given case depends on a variety of factors, such as the nature of the disease, the progress of the disease, the severity of the disease and the particular composition which is used. For example, compositions are administered systemically, for example, via intravenous administration. Subcutaneous methods also can be employed, although increased absorption times can be necessary to ensure equivalent bioavailability compared to intravenous methods.

Pharmaceutical compositions can be formulated in dosage forms appropriate for each route of administration. Pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Unit dosage forms include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared. Pharmaceutical compositions can be formulated in dosage forms appropriate for each route of administration.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. All preparations for parenteral administration must be sterile, as is known and practiced in the art. In some embodiments, the compositions can be provided as a lyophilized powder, which can be reconstituted for administration as solutions, emulsions and other mixtures. They may also be reconstituted and formulated as solids or gels.

The lyophilized powders can be prepared from any of the solutions described above.

The sterile, lyophilized powder can be prepared by dissolving an eIL-15 molecule or complex in a buffer solution. The buffer solution may contain an excipient which improves the stability of other pharmacological components of the powder or reconstituted solution, prepared from the powder.

In some embodiments, subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to ensure sterility and apportioned into vials for lyophilization. Each vial can contain a single dosage (1 mg-1 g, generally 1-100 mg, such as 1-5 mg) or multiple dosages of the compound. The lyophilized powder can be stored under appropriate conditions, such as at about 4° C. to room temperature. Reconstitution of this lyophilized powder with a buffer solution provides a formulation for use in parenteral administration. The precise amount depends upon the indication treated and selected compound. Such amount can be empirically determined.

In some embodiments, the pH of the composition is between or between about 6 and 10, such as between or between about 6 and 8, between or between about 6.9 and 7.3, such as about pH 7.1. In some embodiments, the pH of the pharmaceutically acceptable buffer is at least or about 5, at least or about 6, at least or about 7, at least or about 8, at least or about 9 or at least or about 10, or is 7.1.

The compositions can be formulated for single dosage administration or for multiple dosage administration. The agents can be formulated for direct administration.

In some embodiments, the compositions provided herein are formulated in an amount for direct administration of the eIL-15 molecule or complex, in a range from at or about 0.01 mg to at or about 3000 mg, from at or about 0.01 mg to at or about 1000 mg, from at or about 0.01 mg to at or about 500 mg, from at or about 0.01 mg to at or about 100 mg, from at or about 0.01 mg to at or about 50 mg, from at or about 0.01 mg to at or about 10 mg, from at or about 0.01 mg to at or about 1 mg, from at or about 0.01 mg to at or about 0.1 mg, from at or about 0.1 mg to at or about 2000 mg, from at or about 0.1 mg to at or about 1000 mg, from at or about 0.1 mg to at or about 500 mg, from at or about 0.1 mg to at or about 100 mg, from at or about 0.1 mg to at or about 50 mg, from at or about 0.1 mg to at or about 10 mg, from at or about 0.1 mg to at or about 1 mg, from at or about 1 mg to at or about 2000 mg, from at or about 1 mg to at or about 1000 mg, from at or about 1 mg to at or about 500 mg, from at or about 1 mg to at or about 100 mg, from at or about 1 mg to at or about 10 mg, from at or about 10 mg to at or about 2000 mg, from at or about 10 mg to at or about 1000 mg, from at or about 10 mg to at or about 500 mg, from at or about 10 mg to at or about 100 mg, from at or about 100 mg to at or about 2000 mg, from at or about 100 mg to at or about 1000 mg, from at or about 100 mg to at or about 500 mg, from at or about 500 mg to at or about 2000 mg, from at or about 500 mg to at or about 1000 mg, and from about 1000 mg to at or about 3000 mg. In some embodiments, the volume of the composition can be 0.5 mL to 1000 mL, such as 0.5 mL to 100 mL, 0.5 mL to 10 mL, 1 mL to 500 mL, 1 mL to 10 mL, such as at least or about at least or about or 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL or more.

For example, the composition is formulated for single dosage administration of an amount between at or about 100 mg and at or about 500 mg, or between at or about 200 mg and at or about 400 mg. In some embodiments, the composition is formulated for single dosage administration of an amount between at or about 500 mg and at or about 1500 mg, at or about 800 mg and at or about 1200 mg or at or about 1000 mg and at or about 1500 mg. In some embodiments, the volume of the composition is between at or about 10 mL and at or about 1000 mL or at or about 50 mL and at or about 500 mL; or the volume of the composition is at least at or about 10 mL, 20 mL, 30 mL, 40 mL, 50 mL, 75 mL, 100 mL, 150 mL, 200 mL, 250 mL, 300 mL, 400 mL, 500 mL or 1000 mL.

In some embodiments, the entire vial contents of the formulations can be withdrawn for administration or can be divided up into a plurality of dosages for multiple administrations. Upon withdrawal of an amount of drug for administration, the formulation can be further diluted if desired, such as diluted in water, saline (e.g., 0.9%) or other physiological solution.

In some embodiments, also provided are compositions containing an additional therapeutic agent, such as an immunomodulatory agent, an anti-cancer agent, an anti-viral agent, an antibiotic, an anti-microbial agent, a vaccine, or other therapeutic agent for use in combination with the eIL-15 molecule or complex, in accordance with the provided embodiments. In some aspects, the additional therapeutic agent can be prepared in accord with known or standard formulation guidelines, such as described above. In some embodiments, the therapeutic agent and/or eIL-15 molecule or complex are formulated as separate compositions. In some embodiments, the therapeutic agent is provided as a separate composition from the eIL-15 molecule or complex, and the two compositions are administered separately. In some embodiments, the additional therapeutic agent is provided as a separate composition from the eIL-15 molecule or complex, and the two compositions are administered separately. The compositions can be formulated for parenteral delivery (i.e., for systemic delivery). For example, the compositions or combination of compositions are formulated for subcutaneous delivery or for intravenous delivery. The agents, such as an eIL-15 molecule or complex, and an immunomodulatory agent, an immunomodulatory agent, an anti-cancer agent, an anti-viral agent, an antibiotic, an anti-microbial agent, a vaccine, and/or other therapeutic agent can be administered by different routes of administration.

In some aspects, exemplary additional therapeutic agents, such as immunomodulatory agents, anti-cancer agents, anti-viral agents, antibiotics, anti-microbial agents, vaccines, or other therapeutic agents, can be administered as directed for a monotherapy or on other administration schedules and dose for the particular therapeutic agent. In some embodiments of the methods and uses that involve administration of with an eIL-15 molecule or complex and an additional therapeutic agent, the additional therapeutic agent is administered at the recommended dose and/or schedule of administration. In some embodiments, an additional therapeutic agent can be administered in the methods herein at a dose lower than the recommended amount or on an alternate schedule, such as when eIL-15 molecule or complex sensitizes a disease or condition (such as a tumor, cancer, the TME, infection, or immune response) to the additional therapeutic agent and/or when the combination of an eIL-15 molecule or complex and an additional therapeutic agent results in a synergistic response.

### IV. COMBINATION THERAPY

In some embodiments, also provided are methods and uses that include combination therapies, and combinations, such as combinations for use in accordance with the combination therapy. In some aspects, the combinations include administering an engineered IL-15 (eIL-15) molecule or complex provided herein and an additional therapeutic agent, such as an immunomodulatory agent, an anti-cancer agent, an anti-viral agent, an antibiotic, an anti-microbial agent, a vaccine, and/or other therapeutic agent. In some embodiments, the eIL-15 molecule or complex is administered in conjunction with chemotherapy, Toll-like receptor agonists, or adoptive transfer of tumor reactive CD8+ T cells. In some aspects, the combinations include an eIL-15 molecule or complex provided herein and an additional therapeutic agent, such as an immunomodulatory agent, an anti-cancer agent, an anti-viral agent, an antibiotic, an anti-microbial agent, a vaccine, and/or other therapeutic agent. In some embodiments, the eIL-15 molecule or complex is administered in addition to another treatment regimen, such as photoimmunotherapy (PIT; such as cancer-targeted photoimmunotherapy and/or immune cell-targeted photoimmunotherapy), photodynamic therapy, immunotherapy, radiation, or chemotherapy. In some embodiments, the eIL-15 molecule or complex is administered in addition to another treatment regimen, such as a treatment regimen to combat infection. In some embodiments, the eIL-15 molecule or complex is administered in addition to another treatment regimen to enhance or treat a suppressed immune system. Exemplary PIT that could be used in combination with the eIL-15 molecules or complexes thereof described herein, include, but are not limited to, PIT methods as described in WO 2013/009475, WO 2017/031363, WO 2017/031367, WO 2021/207691, and WO 2022/182483.

In some aspects, the combination therapy includes administration of the eIL-15 molecule or complex and the additional therapeutic is an immunomodulatory agent or an anti-cancer agent. In some of such methods, the primary tumors, newly arising tumors, invasive tumor cells, and metastatic tumor cells can be sensitized to the treatment with the additional therapeutic agent, such as an immunomodulatory agent or an anti-cancer agent. In some of such methods, the eIL-15 molecule or complex can sensitize the primary tumors, newly arising tumors, invasive tumor cells, and/or metastatic tumor cells for improved efficacy of the treatment with the additional therapeutic agent or treatment regimen, such as an immunomodulatory agent or an anti-cancer agent. In any of such methods, the growth of primary tumors, newly arising tumors, invasive tumor cells, and metastatic tumor cells can be inhibited, reduced or eliminated, and/or the volume of one or more tumors is reduced.

The increase in sensitivity as a result of such combination treatments can include, but not limited to, a reduction of inhibition of tumor growth of a primary tumor or a tumor distal to the site of administration, a reduction in tumor cell invasion and/or metastasis, an increase in tumor cell killing, an increase in systemic immune response, an increase in new T cell priming, an increase in the diversity of intratumoral CD8+ T cells, an increase in the number and/or activity of intratumoral CD8+ T effector cells, a decrease in the number and/or activity of intratumoral regulatory T cells, a decrease in the number and/or activity of intratumoral myeloid derived suppressor cells, a decrease in the number and/or activity of intratumoral tumor associated fibroblasts or cancer associated fibroblasts (CAFs), or any combination thereof.

In some embodiments the additional therapeutic agent is an anticancer agent. In some embodiments, the anticancer agent can be one or more chemotherapeutic agent(s), an antibody treatment, and a radiotherapeutic agent. In some embodiments, the additional therapeutic agent is an anti-cancer agent selected from a checkpoint inhibitor, an immune adjuvant, a chemotherapeutic agent, radiation, and a biologic comprising an anti-cancer targeting molecule that binds to a tumor cell.

In some aspects, the additional therapeutic agent is an immunomodulatory agent (also called immune modulating agent), such as an immune checkpoint inhibitor. In some aspects, such combination is employed for treatment of the tumor, lesion or cancer. In some embodiments, the methods include the administration of the immunomodulatory agent, such as an immune checkpoint inhibitor, prior to, concurrent with or subsequent to the administration of an eIL-15 molecule or complex.

In some embodiments, the additional therapeutic agent, such as an immunomodulatory agent, used in such combination therapies herein can include an adjuvant, immune checkpoint inhibitor, cytokine or any combination thereof. A cytokine for use in the combinations can be, for example, Aldesleukin (PROLEUKIN), Interferon alfa-2a, Interferon alfa-2b (Intron A), Peginterferon Alfa-2b (SYLATRON/PEG-Intron), or a cytokine that targets the IFNAR1/2 pathway, the IL-2/IL-2R pathway. An adjuvant for use in the combinations can be, for example, Poly ICLC (HILTONOL/Imiquimod), 4-1BB (CD137; TNFRS9), OX40 (CD134) OX40-Ligand (OX40L), Toll-Like Receptor 2 Agonist SUP3, Toll-Like Receptor TLR3 and TLR4 agonists and adjuvants targeting the Toll-like receptor 7 (TLR7) pathway, other members of the TNFR and TNF superfamilies, other TLR2 agonists, TLR3 agonists and TLR4 agonists.

In some embodiments, the additional therapeutic agent is an immune checkpoint inhibitor that is a PD-1 inhibitor, such as a small molecule, antibody or antigen binding fragment. Exemplary anti-PD-1 antibodies include, but are not limited to, pembrolizumab (MK-3475, Keytruda), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IB1I308), GLS-010, CS1003, LZM009, camrelizumab (SHR-1210), SCT-I10A, MGA012, AK105, PF-06801591, AMP-224, AB122, AMG 404, BI 754091, HLX10, JTX-4014, MEDIO680, Sym021, MGD019, MGD013, AK104, XmAb20717, R07121661, CX-188, and spartalizumab.

In some embodiments, the additional therapeutic agent is an immune checkpoint inhibitor that is a CTLA-4 inhibitor, such as a small molecule, antibody or antigen binding fragment. In some of any embodiments, the anti-CTLA-4 antibody is selected from the group consisting of ipilimumab (YERVOY), tremelimumab, AGEN1181, AGEN1884, ADU-1064, BCD-145, and BCD-217.

In some embodiments, the additional therapeutic agent is a CD25 inhibitor, such as a small molecule, antibody or antigen binding fragment. In some of any embodiments, the anti-CD25 antibody is selected from the group consisting of basiliximab (Simulect®), daclizumab, PC61.

The administration of any of additional therapeutic agent(s) or treatment regimen(s), can be administered prior to, concurrent with, or subsequent to the administration of the eIL-15 molecule or complex.

### V. ARTICLES OF MANUFACTURE OR KITS

Also provided are articles of manufacture or kit containing the provided eIL-15 molecules or complexes, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include intravenous solution bags, vials, including those with stoppers pierceable by a needle for injection. The article of manufacture or kit may further include a package insert indicating that the compositions can be used to treat a particular condition such as a condition described herein (e.g., multiple myeloma). Alternatively, or additionally, the article of manufacture or kit may further include another or the same container comprising a pharmaceutically acceptable buffer. It may further include other materials such as other buffers, diluents, filters, needles, and/or syringes.

The label or package insert may indicate that the composition is used for treating a disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a disorder or condition in an individual by eIL-15 administration.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the eIL-15 molecule or complex; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VI. DEFINITIONS

An “isolated” eIL-15 molecule is one which has been separated from environment in which the eIL-15 molecule was manufactured (e.g., host cell). In some embodiments, an eIL-15 molecule or complex is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an eIL-15 complex” refers to one or more nucleic acid molecules encoding an engineered IL-15 molecule provided herein and all or at least a portion of the alpha subunit of the IL-15R (e.g., sushi domain of IL-15Rα), optionally linked to an Fc domain, including such nucleic acid molecule(s) in a single vector or separate vectors, and such nucleic acid molecule(s) present at one or more locations in a host cell.

As used herein, “complex” refers to the joining or linking together of two or more molecules resulting in the formation of another entity, by any known joining or linking methods or interactions. For example, an engineered IL-15 polypeptide joined or linked, directly or indirectly, to, or interacts with, one or more chemical moieties or a second polypeptide is an exemplary complex. Such complexes include those where the joining, linking, or interaction is covalent or non-covalent, and may include fusion proteins, those produced by chemical conjugates and those produced by any other methods. In some cases, non-covalent and covalent linkages or interactions may join different molecules of more than two molecules in a complex. For example, an eIL-15:IL-15Ra complex provided herein can include an engineered IL-15 polypeptide that is non-covalently linked to the sushi domain of IL-15 receptor alpha (IL-15Ra), in which the sushi domain is also fused covalently to an Fc domain of an immunoglobulin, such as via a linker.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The terms “polypeptide” and “protein” are used interchangeably to refer to a polymer of amino acid residues and are not limited to a minimum length. Polypeptides, including the eIL-15 molecules or complexes and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity.

These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Len, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### VII. EXEMPLARY EMBODIMENTS

Among the provided embodiments are:
1. An engineered IL-15 polypeptide comprising at least 6 cysteine residues and capable of forming at least 3 intramolecular disulfide bonds.
2. The engineered IL-15 polypeptide of embodiment 1, wherein the IL-15 polypeptide sequence is derived from a mammalian IL-15.
3. The engineered IL-15 polypeptide of any one of embodiments 1, wherein the IL-15 polypeptide sequence is derived from a human IL-15.
4. The engineered IL-15 polypeptide of embodiment 1, comprising two amino acid substitutions in SEQ ID NO: 2, wherein the two amino acid substitutions are substituting a non-cysteine residue with a cysteine.
5. The engineered IL-15 polypeptide of any one of embodiments 1-4, wherein two of the cysteine residues are present at a position corresponding to positions 24 and 93 of SEQ ID NO:2 or corresponding to positions 29 and 102 of SEQ ID NO:2.
6. The engineered IL-15 polypeptide of any one of embodiments 1-5, comprising at least one amino acid substitution at a position corresponding to position 4, 10, 11, 14, 17, 18, 20, 24, 29, 32, 34, 36, 41, 52, 57, 58, 77, 80, 83, 93, 97, 102, 105, 111, or 112 of SEQ ID NO: 2.
7. The engineered IL-15 polypeptide of any one of embodiments 1-6, wherein the engineered IL-15 polypeptide comprises up to 2, 3, 4, 5, 6, 7, 8, 9, 10, 11, 12, 13, 14, 15, 16, 17, 18, 19 or 20 amino acid substitutions as compared to SEQ ID NO:2.
8. The engineered IL-15 polypeptide of any one of embodiments 1-7, wherein the engineered IL-15 polypeptide comprises one or more amino acid substitutions selected from the group consisting of N4D, N4E, K10R, K11Y, K11E, Q17S, S18N, H20N, T24C, T24L, S29C, H32N, H32S, S34K, S34R, K36S, K41L, L52R, A57P, S58P, S58Q, N77S, V80K, S83D, E93C, K97A, S102C, H105W, I111A, and N112L, with reference to positions of SEQ ID NO:2.
9. The engineered IL-15 polypeptide of embodiment 8, comprising 2, 3, 4, 5, 6, 7, 8, 9, 10, 11, 12, 13, 14, or 15 amino acid substitutions selected from the group consisting of N4D, N4E, K10R, K11Y, K11E, Q17S, S18N, H20N, T24C, T24L, S29C, H32N, H32S, S34K, S34R, K36S, K41L, L52R, A57P, S58P, S58Q, N77S, V80K, S83D, E93C, K97A, S102C, H105W, I111A, and N112L, with reference to positions of SEQ ID NO:2.
10. The engineered IL-15 polypeptide of any one of embodiments 1-9, wherein the engineered IL-15 polypeptide further comprises the amino acid substitution N72D, with reference to positions of SEQ ID NO:2.
11. The engineered IL-15 polypeptide of any one of embodiments 1-10, wherein the engineered IL-15 polypeptide has at least 75% identity and less than 90% identity to SEQ ID NO:2.
12. The engineered IL-15 polypeptide of any one of embodiments 1-5, wherein the engineered IL-15 polypeptide comprises one or more amino acid substitution in helix A, helix B, helix C, helix D of IL-15, or any combination thereof.
13. The engineered IL-15 polypeptide of any one of embodiments 1-5, wherein the engineered IL-15 polypeptide comprises one or more amino acid substitution in the loop region between helix A and helix B, between helix B and helix C, between helix C and helix D of IL-15, or any combination thereof.
14. The engineered IL-15 polypeptide of any one of embodiments 1-5, wherein the engineered IL-15 polypeptide comprises the addition of a cysteine in the loop region between helix A and helix B and/or between helix C and helix D of IL-15.
15. The engineered IL-15 polypeptide of embodiment 14, wherein the addition of the cysteine comprises a substitution of a cysteine for another amino acid in SEQ ID NO:2.
16. A complex, comprising the engineered IL-15 polypeptide of any one of embodiments 1-15 and a second polypeptide.
17. The complex of embodiment 16, wherein the second polypeptide comprises an antibody or antigen-binding fragment.
18. The complex of embodiment 16, wherein the second polypeptide comprises an Fc domain or a portion thereof.
19. The complex of embodiment 16, wherein the second polypeptide comprises a receptor molecule or domain thereof.
20. The complex of embodiment 19, wherein the second polypeptide comprises an IL-15 receptor molecule or domain thereof.
21. The complex of embodiment 19, wherein the second polypeptide comprises an IL-15 receptor sushi domain.
22. The complex of embodiment 16, wherein the second polypeptide comprises a receptor molecule or domain thereof fused to an Fc domain or a portion thereof.
23. The complex of embodiment 22, wherein the second polypeptide comprises an IL-15 receptor molecule or domain thereof fused to an Fc domain or a portion thereof.
24. The complex of embodiment 22 or 23, wherein the second polypeptide comprises an IL-15 sushi domain fused to an Fc domain.
25. A complex, comprising the engineered IL-15 polypeptide of any one of embodiments 1-15 and a second polypeptide comprising an IL-15 sushi domain fused to an Fc domain.
26. The complex of embodiment 24 or 25, wherein the second polypeptide comprises the amino acid sequence set forth in SEQ ID NO:41, or an amino acid sequence that has at least 90%, 91%, 92%, 93%, 94%, 95%, 96%, 97%, 98%, or 99% sequence identity to SEQ ID NO:41.
27. The complex of any one of embodiments 16-26, wherein the engineered IL-15 polypeptide and the second polypeptide are linked non-covalently.
28. The complex of any one of embodiments 16-26, wherein the engineered IL-15 polypeptide and the second polypeptide are linked covalently.
29. The complex of any one of embodiments 16-28, wherein the complex further comprises a phthalocyanine dye.
30. The complex of embodiment 29, wherein the phthalocyanine dye is covalently linked to the second polypeptide.
31. A nucleic acid molecule encoding the engineered IL-15 polypeptide of any one of embodiments 1-15, or the complex of any one of embodiments 16-30.
32. A vector comprising the nucleic acid molecule of embodiment 31.
33. The vector of embodiment 32, wherein the vector is an expression vector.
34. The vector of embodiment 32 or 33, wherein the vector is a mammalian vector or a viral vector.
35. A cell comprising the engineered IL-15 polypeptide of any one of embodiments 1-15 or the complex of any one of embodiments 16-30.
36. A cell comprising the nucleic acid molecule of embodiment 31 or the vector of any one of embodiments 32-34.
37. The cell of embodiment 27 or 28, wherein the cell is a mammalian cell.
38. A pharmaceutical composition comprising the engineered IL-15 polypeptide of any one of embodiments 1-15, or the complex of any one of embodiments 16-30.
39. A method for treating a disease or condition, the method comprising administering the engineered IL-15 polypeptide of any one of embodiments 1-15 in conjunction with an IL-15 receptor or functional domain thereof.
40. The method for embodiment 39, wherein the engineered IL-15 polypeptide is administered in conjunction with a second polypeptide comprising an IL-15 receptor molecule or domain thereof fused to an Fc domain or a portion thereof.
41. The method for embodiment 40, wherein the second polypeptide comprises an IL-15 sushi domain fused to an Fc domain.
42. The method for embodiment 40 or 41, wherein the second polypeptide comprises the amino acid sequence set forth in SEQ ID NO:41, or an amino acid sequence that has at least 90%, 91%, 92%, 93%, 94%, 95%, 96%, 97%, 98%, or 99% sequence identity to SEQ ID NO:41.
43. A method for treating a disease or condition, the method comprising administering the engineered IL-15 polypeptide of any one of embodiments 1-15, the complex of any one of embodiments 16-30, or the pharmaceutical composition of embodiment 38.
44. The method of any one of embodiments 39-43, wherein the method further comprises administering a second agent.
45. The method for embodiment 34, wherein the second agent is selected from radiation, photoimmunotherapy, chemotherapy, an immune checkpoint inhibitor, a tyrosine kinase inhibitor, a CAR-T cell, or a CAR-NK cell.
46. A method for treating a disease or condition in a subject, the method comprising administering the engineered IL-15 polypeptide of any one of embodiments 1-15, and a photoimmunotherapy.
47. A method for treating a disease or condition in a subject, the method comprising administering the complex of any one of embodiments 16-30, and a photoimmunotherapy.
48. A method for treating a disease or condition in a subject, the method comprising administering the pharmaceutical composition of embodiment 38, and a photoimmunotherapy.
49. The method for any one of embodiments 45-48, wherein the photoimmunotherapy comprises:
(a) intravenously administering to the subject a targeting conjugate comprising a silicon phthalocyanine dye linked to a targeting molecule, wherein the targeting molecule is capable of binding to a target on the surface of a target cell; and
(b) after administering the targeting conjugate, irradiating an area around or near a target cell at a wavelength and dose sufficient to kill the target cell, thereby treating the disease or condition.
50. The method for any one of embodiments 45-49, wherein the engineered IL-15 polypeptide, the complex or the pharmaceutical composition is administered prior to, concurrent with or subsequent to the photoimmunotherapy.
51. The method for embodiment 49 or 50, wherein the target cell is a tumor cell, a cell present in the tumor microenvironment, or an immune cell.
52. The method for any one of embodiments 49-51, wherein the targeting molecule is capable of binding to Treg cells.
53. The method for any one of embodiments 49-51, wherein the targeting molecule is capable of binding to PD-L1 or PD-1.
54. The method for any one of embodiments 49-51, wherein the targeting molecule is capable of binding to EGFR.
55. A method for modulating an immune response in a subject, comprising administering the engineered IL-15 polypeptide of any one of embodiments 1-15, the complex of any one of embodiments 16-30, or the pharmaceutical composition of embodiment 38 to a subject.
56. The method for embodiment 55, wherein modulating the immune response treats a disease or condition in the subject.
57. The method for any one of embodiments 39-54 and 56, wherein the disease or condition is selected from the group consisting of a cancer, a tumor, an infection, a viral infection, an immunocompromised state, and an immune deficiency.
58. The method for embodiment 55, wherein modulating the immune response increases the immune response to vaccination.
59. The method for any one of embodiments 55-58, wherein the immune response is an increase is one or more immune modulating molecules in the treated subject as compared to prior to the treatment.

### VIII. EXAMPLES

The following examples are included for illustrative purposes only and are not intended to limit the scope of the invention.

### Example 1: Generation of Novel Engineered IL-15 (eIL-15) Molecules

Exemplary engineered human IL-15 (eIL-15) molecules were generated and assessed.

A. Generation of eIL-15 Molecules with Single Amino Acid Substitutions

Novel human IL-15 molecules were generated by analyzing the crystal structure of IL-15 in complex with its receptor, IL-15Rα:IL-2Rβ:IL-2Rγ (PDB: 4GS7), and making single amino acid substitutions at the following positions corresponding to the positions in the amino acid sequence of mature wild-type human IL-15 set forth in SEQ ID NO: 2: V3I (SEQ ID NO: 4), V3W (SEQ ID NO: 5), N4H (SEQ ID NO: 6), 16L (SEQ ID NO: 7), K10R (SEQ ID NO: 8), K10Y (SEQ ID NO: 9), K11E (SEQ ID NO: 10), K11Y (SEQ ID NO: 11), A23P (SEQ ID NO: 12), T24P (SEQ ID NO: 13), L45I (SEQ ID NO: 14), E46R (SEQ ID NO: 15), V49W (SEQ ID NO: 16), A57P (SEQ ID NO: 17), S58P (SEQ ID NO: 18), N72T (SEQ ID NO: 19), H105D (SEQ ID NO: 20), H105E (SEQ ID NO: 21), H105W (SEQ ID NO: 22), N112F (SEQ ID NO: 23), N112L (SEQ ID NO: 24), N112Y (SEQ ID NO: 25).

His-tagged IL-15 molecules (IL-15-6His) with the above expressed in mammalian cells, such as ExpiCHO-S cells (Gibco Thermo Fisher Scientific), together with an IL-15Rα-human Fc chimeric protein (hFc) (SEQ ID NO: 41) by transient transfection using a DNA ratio of 2:1 (IL-15-6His:IL-15a-hFc). Protein complexes were purified by nickel chromatography and analyzed by SEC-HPLC to confirm purity.

B. Generation of eIL-15 Molecules Capable of Forming Novel Disulfide Bond

Novel human IL-15 molecules were generated by analyzing the crystal structure of IL-15 in complex with its receptor IL-15Rα:IL-2Rβ:IL-2Rγ (PDB: 4GS7) and substituting two amino acids with cysteine to introduce the capability of forming a novel disulfide bond into the tertiary structure of the mature human IL-15 protein amino acid sequence set forth in SEQ ID NO: 2, thereby increasing the structural rigidity of the molecules generated. The substituted pairs included replacing the glutamic acid corresponding to position 13 and the leucine at position 100 of SEQ ID NO: 2 with cysteine (E13C/L100C; SEQ ID NO: 26), replacing the leucine corresponding to position 15 and the isoleucine at position 59 of SEQ ID NO: 2 with cysteine (L15C/I59C; SEQ ID NO: 27), replacing the threonine corresponding to position 24 and glutamic acid at position 93 of SEQ ID NO: 2 with cysteine (T24C/E93C; SEQ ID NO: 28), or replacing the serines corresponding to positions 29 and 102 of SEQ ID NO: 2 with cysteine (S29C/S102C; SEQ ID NO: 29).

In addition to the cysteine pair substitutions, additional amino acid substitutions were selected from the following: N4D, N4E, K10R, K11Y, K11E, D14S, D14N, Q17S, S18N, H20N, T24L, T24P, H32N, H32S, S34K, S34R, K36S, K41L, L52R, A57P, N77S, S58P, S58Q, V80K, S83D, K97A, H105W, I111 A, N112L, corresponding to the positions in the amino acid sequence set forth in SEQ ID NO: 2. The additional substitutions were selected based on experimental or predicted proliferation stimulated by the mutants using assays described in Example 2. Exemplary combinations of the mutations in the generated eIL-15 molecules are provided in Table E1 below.

TABLE E1

| | | SEQ |
|---|---|---|
| eIL-15 | Mutations | ID NO |
| eIL-15-A | K10R/K11Y/D14S/Q17S/H20N/T24C/S34K/K36S/K41L/A57P/V80K/S83D/E93C | 30 |
| eIL-15-B | K10R/K11Y/L15C/Q17S/S18N/T24P/S34K/K36S/K41L/A57P/I59C/H105W/ | 31 |
| | N112L | |
| eIL-15-C | K10R/K11E/D14S/S18N/T24L/S29C/S34K/S58P/V80K/S83D/S102C/H105W/ | 32 |
| | N112L | |
| eIL-15-D | K10R/K11E/D14S/Q17S/H20N/T24C/S34K/K36S/V80K/S83D/E93C/K97A | 33 |
| eIL-15-E | K10R/K11E/D14S/Q17S/H20N/T24C/S34K/K36S/V80K/S83D/E93C/H105W | 34 |
| eIL-15-F | K10R/K11E/D14S/Q17S/T24C/S34K/K36S/V80K/S83D/E93C/K97A/H105W | 35 |
| eIL-15-G | K10R/K11E/D14N/Q17S/H20N/T24C/S58Q/V80Y/S83D/E93C/K97A/H105W | 36 |
| eIL-15-H | K10R/K11E/D14N/H20N/T24C/S34K/K36S/V80K/S83D/E93C/K97A/H105W | 37 |
| eIL-15-I | K10R/K11Y/D14S/Q17S/H20N/T24C/L52R/V80K/S83D/E93C/K97A/N112L | 38 |
| eIL-15-J | N4D/K10R/K11E/D14N/Q17S/T24C/H32N/S34R/K36S/N77S/E93C/I111A | 39 |
| eIL-15-K | N4E/K10R/K11E/D14N/Q17S/T24C/H32S/S34R/K36S/N77S/E93C/I111A | 40 |

The IL-15 molecules were expressed in mammalian cells, such as ExpiCHO-S cells (Gibco Thermo Fisher Scientific), together with an IL-15Ra-human Fc chimeric protein (hFc) (SEQ ID NO: 41) by transient transfection using a DNA ratio of 2:1 (IL-15 molecule:IL-15a-hFc). Protein complexes were purified on a protein A column and analyzed by SEC-HPLC. E13C/L100C, L15C/I59C, and T24C/E93C eIL-15 molecules were His-tagged and purified by nickel chromatography. The analysis of exemplary eIL-15 molecules is provided in Table E2 below. The identities of the eIL-15 molecules and IL 15Rα-hFc molecules were confirmed by intact mass analysis.

TABLE E2

| | | Concen- | | | | |
|---|---|---|---|---|---|---|
| | Volume | tration | Total | SEC analysis | | |
| eIL-15 | (mL) | (g/L) | mass | HMW % | Main % | LMW % |
| eIL-15-A | 8 | 1.57 | 12.6 | 2.3 | 89.0 | 8.7 |
| eIL-15-C | 8 | 1.19 | 9.6 | 3.6 | 87.1 | 9.2 |

### Example 2: Binding Kinetics of eIL-15 Complexes to hIL-2Rβ:IL-2Rγ

Binding affinities of exemplary human IL-15 variant complexes generated in Example 1B were determined by Bio-Layer Interferometry (BLI) using the Sartorius Octet® system. Wild-type IL-15 and IL-15 with a substitution of aspartic acid for asparagine at position 72 (IL-15_N72D), in complex with IL-15Rα-hFc, were also expressed and purified from mammalian cells, and tested as reference molecules. Briefly, the hIL-15 complexes were captured by anti-human IgG that were immobilized on an Octet optical tip. The tips were then immersed in solutions containing various serial dilutions (1:2) to 50 mM hIL-2Rβ:IL-2Rγ dimers (ACROBiosystems Cat. No. ILG-H5283) in PBS, 0.5% tween 20 at 25° C. The on rates of the interactions were calculated from the binding traces and the off rates were determined by fitting the dissociation data obtained in PBS, 0.05% tween 20 alone. Reference wells were used to subtract background noise. The binding kinetics are summarized in Table E2.

TABLE E2
Kinetic and Affinity Summary at 25° C.

| Parameter | wt_IL-15 | IL-15_N72D | eIL-15-A | eIL-15-C |
|---|---|---|---|---|
| kₐ (M⁻¹s⁻¹) | 3.78E+05 | 3.89E+05 | 3.62E+05 | 3.49E+05 |
| k_{d} (s) | 3.48E−05 | 2.97E−05 | 1.26E−04 | 2.37E−04 |
| K_{D} (M) | 9.22E−11 | 7.65E−11 | 3.47E−10 | 6.78E−10 |

### Example 3: Cell Proliferation

A. Proliferation of Murine T Cells by Single or Double Amino Acid-Substituted eIL-15 Molecules

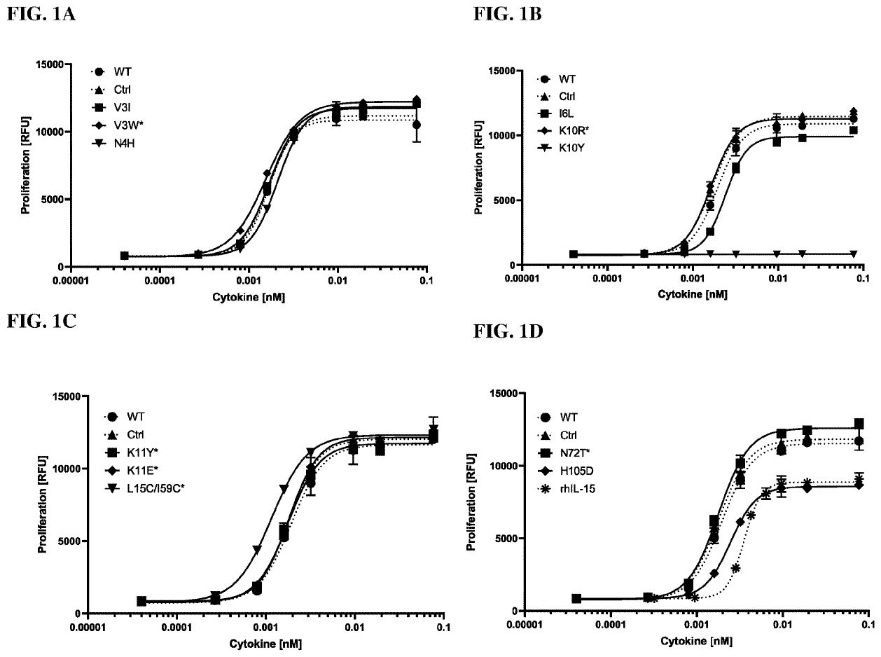

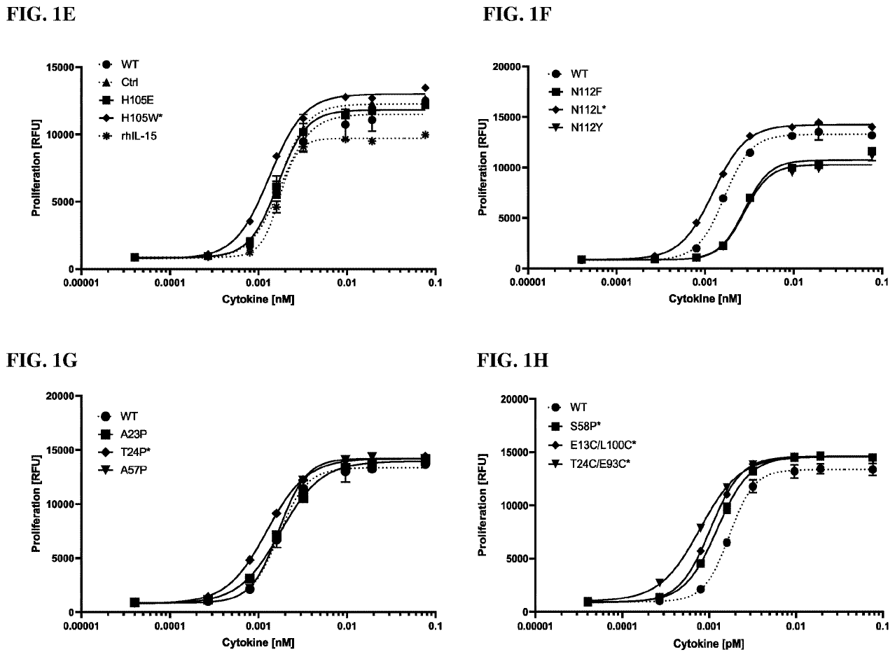

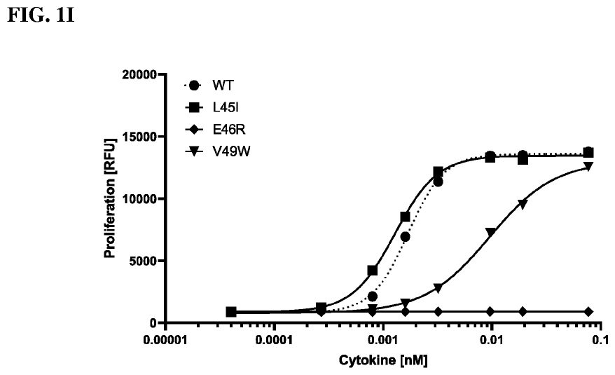

Murine T cells, CTLL2, which express all three subunits of IL-15R, were grown in complete cell culture medium (RPMI-1650, 10% FBS), supplemented with T-STIM, 1 mM sodium pyruvate, and glucose (2.5 g/L). The medium was removed, and the cells were washed twice with HBSS, then incubated for 4 hours in assay medium (RPMI-1650, 5% FBS, 1 mM sodium pyruvate, 2.5 g glucose/L). The cells were then washed twice in PBS and seeded at 30,000 cells/well (100 uL/well) in a 96 well plate in assay medium. The cells were grown for 3 days in the presence of purified His-tagged IL-15 protein complexes containing single, double mutations described in Example 1 (SEQ ID NOS: 4-25) at final concentrations ranging from 0.3 pM to 77 pM. His-tagged IL-15 complexes containing the wild-type (SEQ ID NO: 2) and/or control IL-15 (SEQ ID NO: 3) complex were also tested for comparison. Prior to measurement, the cells were incubated 18 hrs (overnight) with 30 μL/well PRESTO BLUE (viability reagent; Thermo Fisher Cat. No. A-13262). Proliferation was assessed by measuring the fluorescence at 590(±10) nm following excitation at 560(±10) nm (FIGS. 1A-1I).

Several of the single mutations stimulated more cell proliferation than the wild-type or control IL-15 molecules. Some exhibited similar stimulatory activity and others exhibited reduced activity. IL-15 molecules containing E13C/L100C, L15C/I59C, or T24C/E93C mutations, possibly introducing a third intramolecular disulfide bond stimulated increased proliferation compared to wild-type and/or control sequences (FIG. 1C and FIG. 1H). Due to the increase proliferative activity, several of the tested amino acid substitutions were selected for incorporation into further substituted IL-15 molecules and evaluated for activity.

B. Proliferation of Murine T Cells by Multiply Substituted eIL-15 Molecules

CTLL2 cells were assayed for proliferation, using the protocol substantially as described in Example 3A, incubating the CTLL2 cells with recombinant human IL-15 (rIL-15; R&D Systems Cat. No. 247-ILB-025/CF); purified His-tagged IL-15 (T24C/E93C) complex; purified eIL-15 protein complexes containing eIL-15-A (SEQ ID NO: 30), eIL-15-B (SEQ ID NO: 31) or eIL-15-C(SEQ ID NO: 32); or purified control IL-15 (SEQ ID NO: 3) complex.

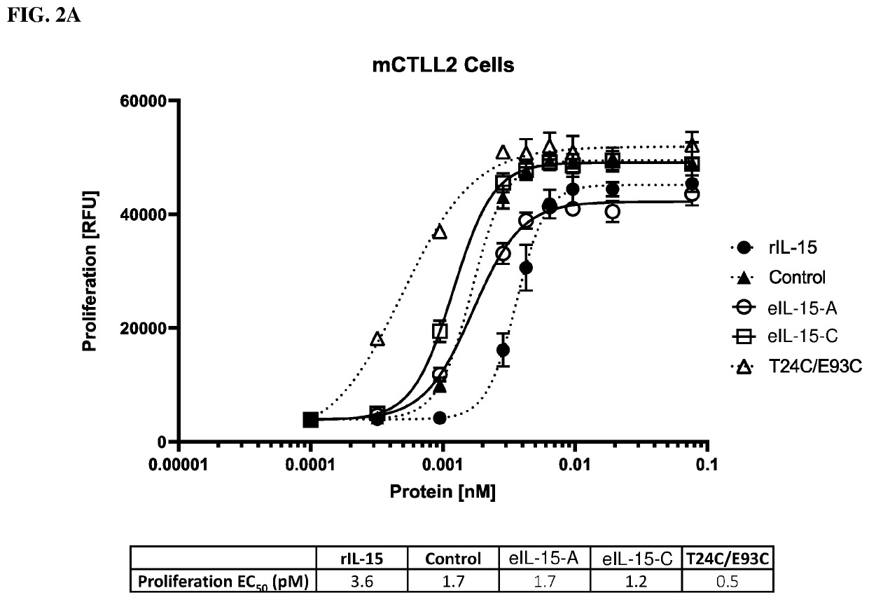

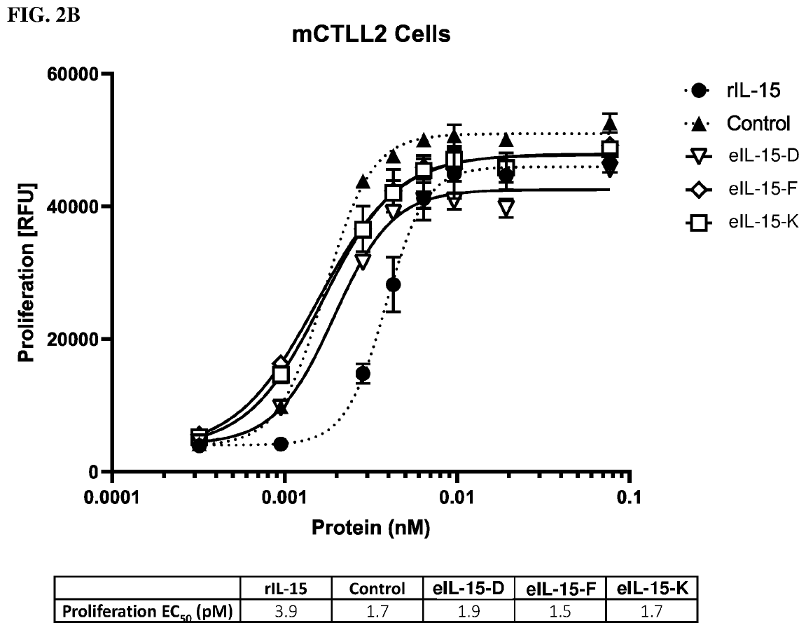

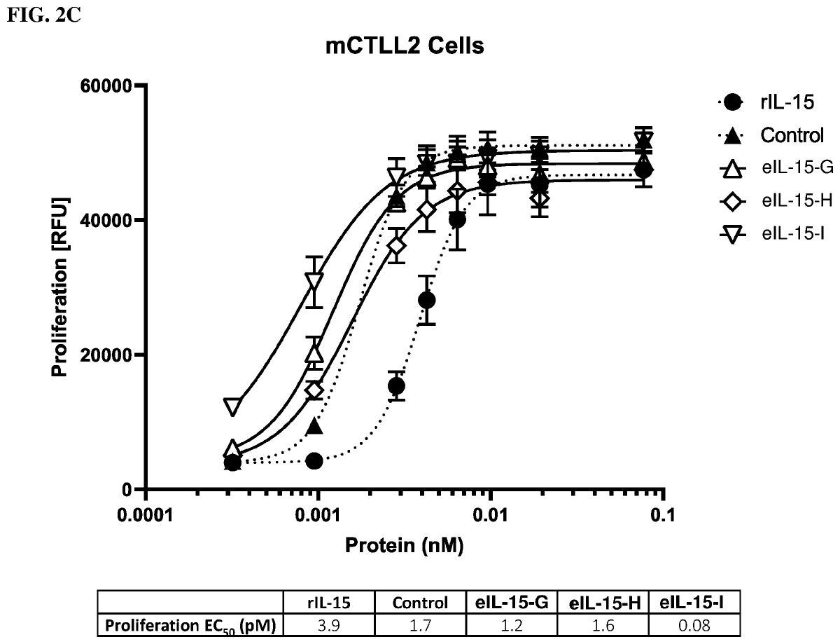

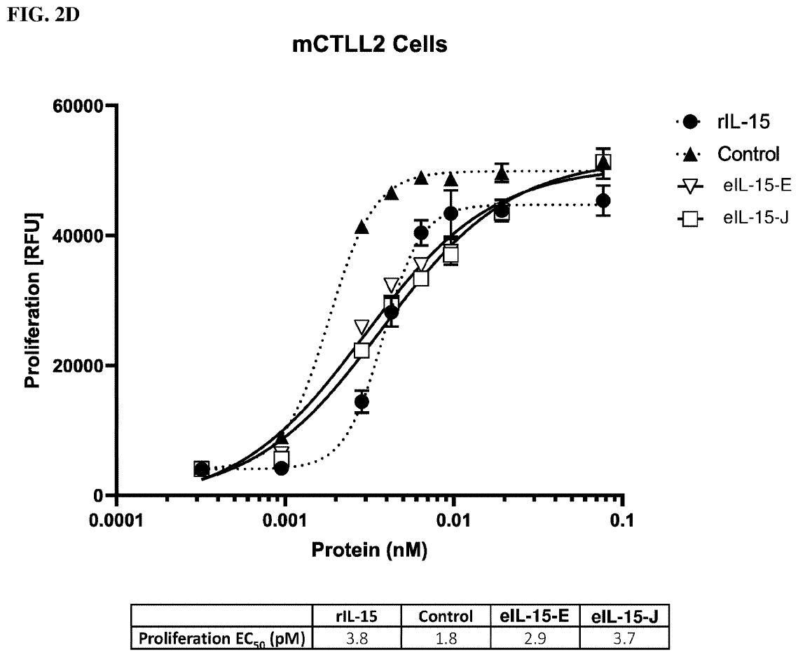

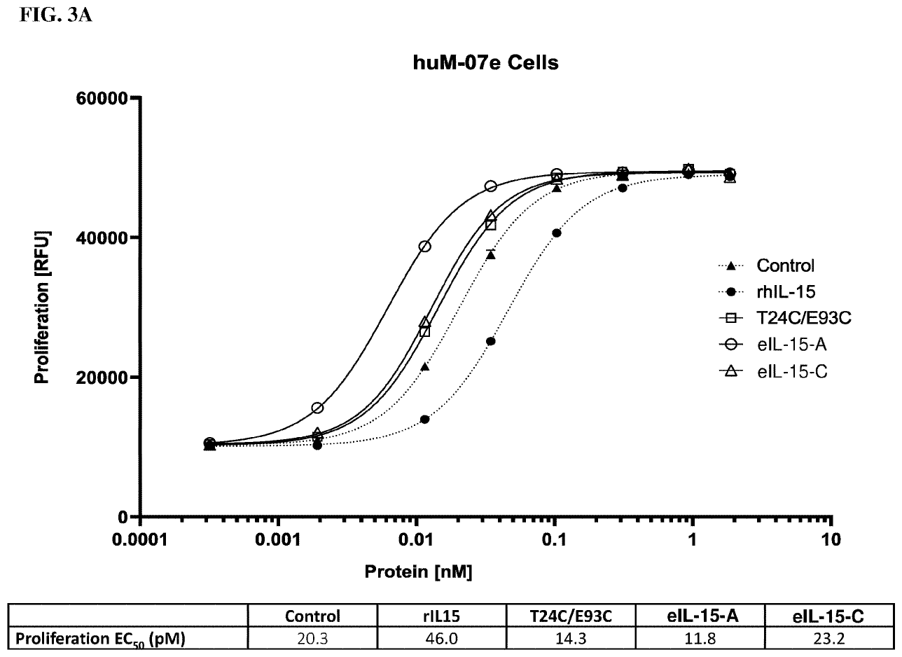

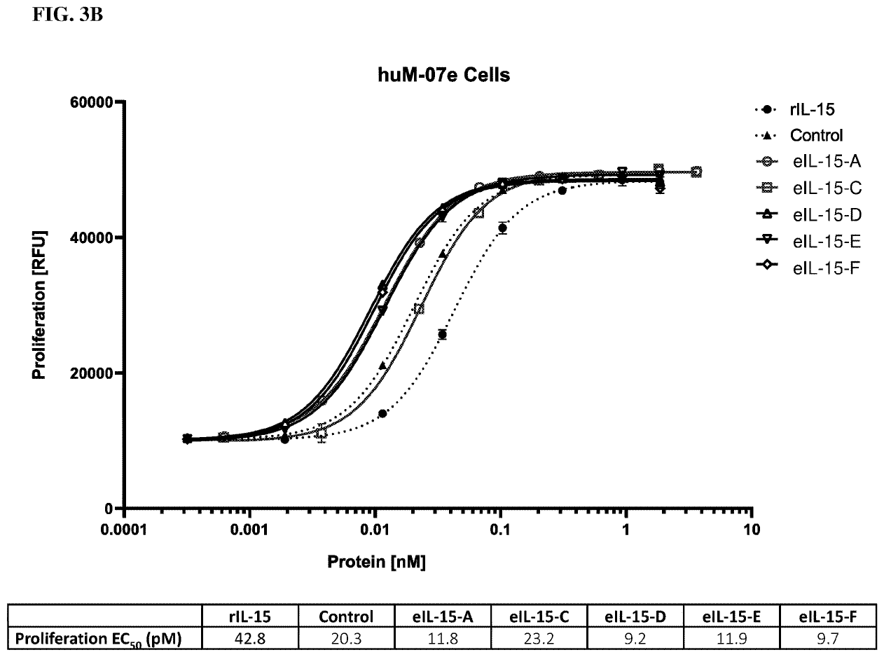

The proliferation and EC₅₀ results are shown in FIG. 2A. The proliferation stimulated by incubation with the T24C/E93C complex (open triangles; EC₅₀: 0.5 pM) or eIL-15-C complex (open squares; EC₅₀: 1.2 pM) was increased compared to the control IL-15 molecule (closed triangles; EC₅₀: 3.6 pM) and rIL-15 (closed circles; EC₅₀: 1.7 pM). The eIL-15-A complex (open circles; EC₅₀: 1.7 pM) promoted similar cell proliferation as the control IL-15 complex (closed triangles) and increased proliferation compared to rIL-15 (closed circles). The eIL-15-B complex negligibly promoted cell proliferation (data not shown).

In a further experiment, CTLL2 cell proliferation was measured, using the previously described procedure, incubating the cells with purified eIL-15 protein complexes containing eIL-15-D (SEQ ID NO: 33), eIL-15-E (SEQ ID NO: 34), eIL-15-F (SEQ ID NO: 35), eIL-15-G (SEQ ID NO: 36), eIL-15-H (SEQ ID NO: 37), eIL-15-I (SEQ ID NO: 38), eIL-15-J (SEQ ID NO: 39), eIL-15-K (SEQ ID NO: 40); rIL-15; or purified control IL-15 (SEQ ID NO: 3) complex. As shown in FIGS. 2B-2D, each of the purified eIL-15 complexes tested promoted dose-dependent CTLL2 cell proliferation.

C. Proliferation of Cells Expressing hIL-2Rβγ

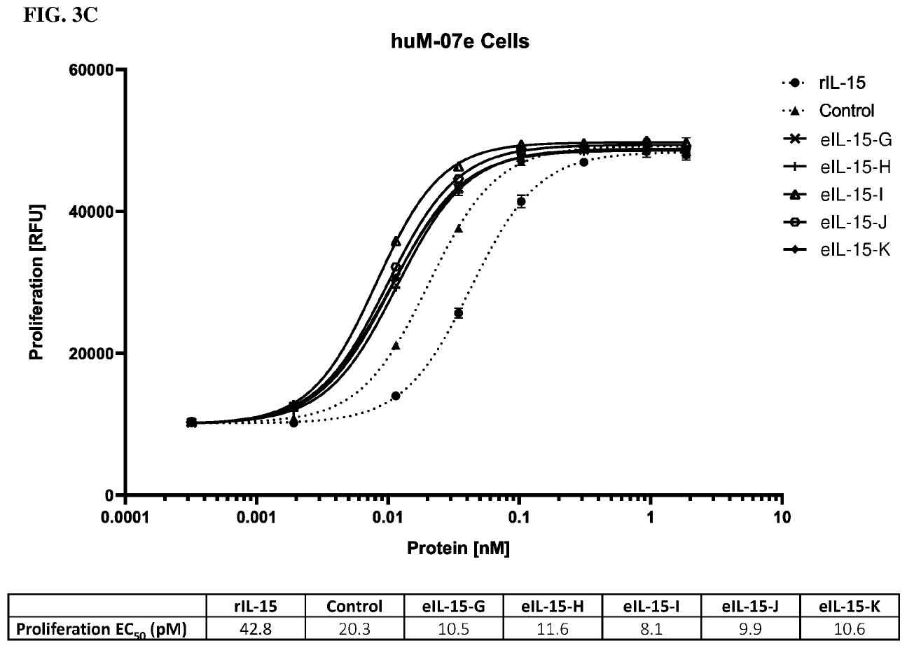

Human megakaryoblastic leukemia M-07e cells (Creative Bioarray; Cat. No. CSC-C0249) were grown in complete cell medium (RPMI-1650, 20% FBS), supplemented with 10 ng/mL granulocyte-macrophage colony-stimulating factor (GM-CSF). The medium was removed, and the cells were washed twice with HBSS, then incubated for 4 hours under starvation conditions (RPMI-1650, low (2%) FBS, without any additional supplements). The cells were then washed with PBS and seeded 30,000 cells/well (100 μL/well) in a 96-well plate in assay medium (RPMI-1650, 2% FBS, 1 mM sodium pyruvate, 2.5 g/L glucose) in the presence of 8 concentrations, ranging from 1.9 to 0.0003 nM or 3.6 to 0.006 nM, of purified exemplary IL-15 complexes containing eIL-15-A, eIL-15-C, eIL-15-D, eIL-15-E, eIL-15-F, eIL-15-G, eIL-15-H, eIL-15-I, eIL-15-J, eIL-15-K, and His-tagged T24C/E93C for 3 to 4 days. Control IL-15 (SEQ ID NO: 3) complex and recombinant human IL-15 (rIL-15) were also tested for comparison. Prior to assay, the cells were incubated 18 hrs (overnight) with 30 μL/well PRESTO BLUE (viability reagent; Thermo Fisher Cat. No. A-13262). Proliferation was assessed by measuring the fluorescence at 590(±10) nm following excitation at 560(±10) nm. As shown in FIGS. 3A-3C, all mutants (solid line) tested stimulated more proliferation of M-07e cells than either the control IL-15 complex and/or recombinant IL-15 (closed symbols, dotted lines).

### D. Proliferation of Activated Primary Human CD8+ T Cells

Primary human CD8⁺ T cells (Cellero) were expanded and frozen in aliquots according to vendor instructions. Cell aliquots were rapidly thawed, pelleted and resuspended in ImmunoCult™-XF T Cell Expansion Medium (StemCell Technologies) to a concentration of 0.5-1×10⁵ cells/mL. The cells were then expanded in the presence of 25 μL/mL ImmunoCult™ Human CD3/CD28 T Cell Activator Factor (AF) and ˜3000UI recombinant human IL-2 (rhu-IL-2) for 2 passages (˜5-7 days). After the second passage, the cells were adapted to grow in rhu-IL-2 (300UI) growth media, without AF.

The further expanded CD8⁺ T cells were washed twice in HBSS and then incubated in culture medium without any growth factor at 37° C. and 5% CO₂ for 4 hours. The cells were then washed twice in PBS and seeded at 30,000 cells/well (100 μL/well) in a 96-well plate. The cells were grown in the presence of 0.0005 nM to 2 nM or 0.0009 to 3.9 nM of purified exemplary IL-15 complexes containing eIL-15 molecules T24C/E93C, eIL-15-A, eIL-15-B, eIL-15-C, eIL-15-D, eIL-15-E, eIL-15-F, eIL-15-G, eIL-15-H, eIL-15-I, eIL-15-J, and eIL-15-K for 4 days. Control IL-15 (SEQ ID NO: 3) complex and recombinant human IL-15 (rIL-15) were also tested for comparison. Prior to measurement, the cells were incubated 18 hrs (overnight) with 30 μL/well PRESTO BLUE (viability reagent; Thermo Fisher Cat. No. A-13262). Proliferation was assessed by measuring the fluorescence at 590(±10) nm following excitation at 560(±10) nm.

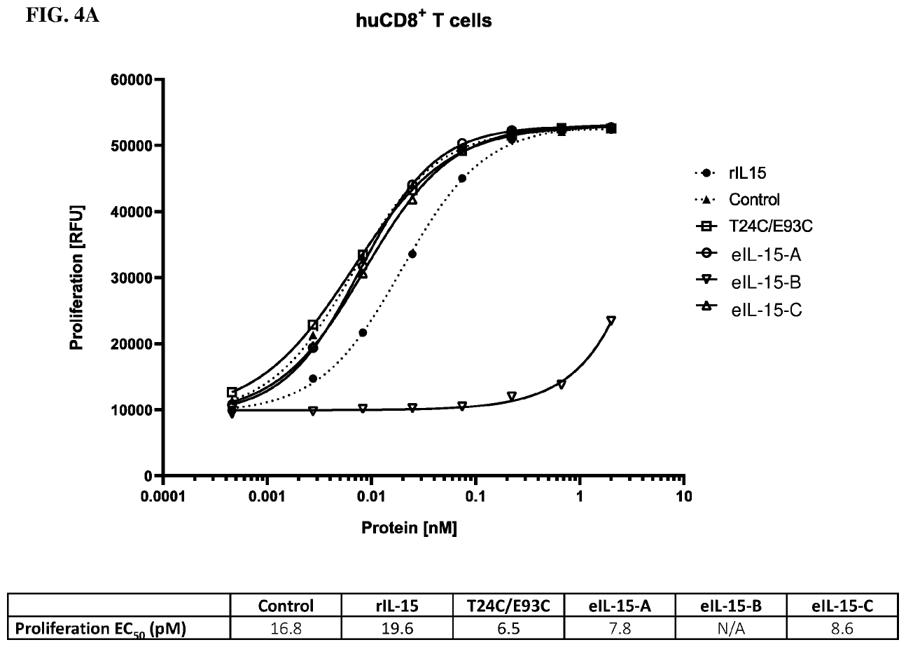

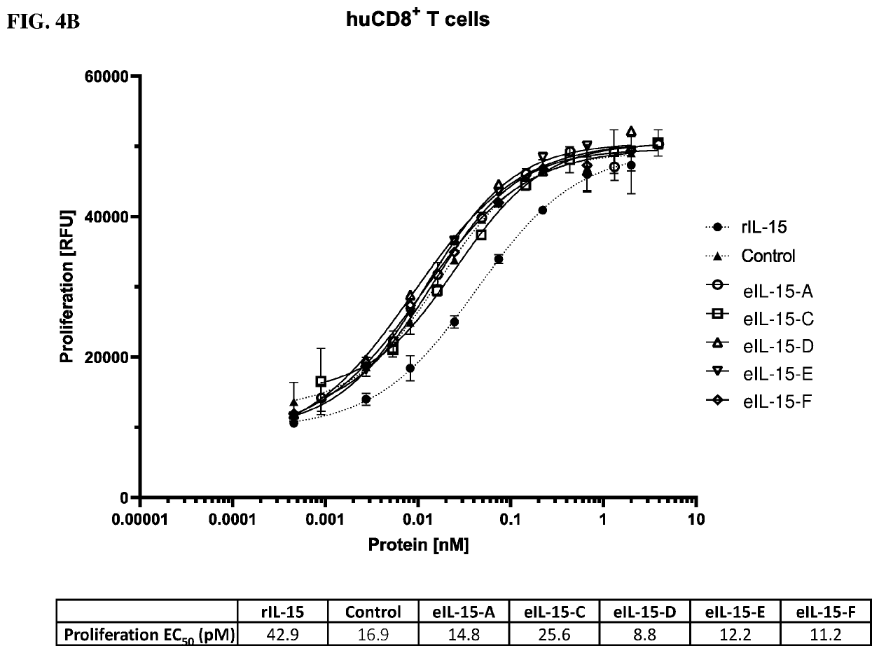

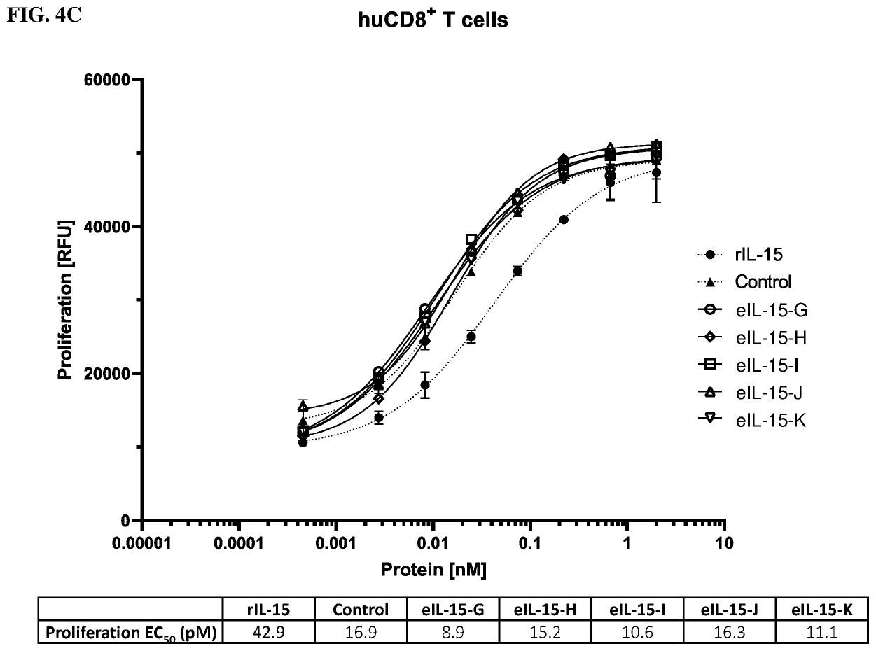

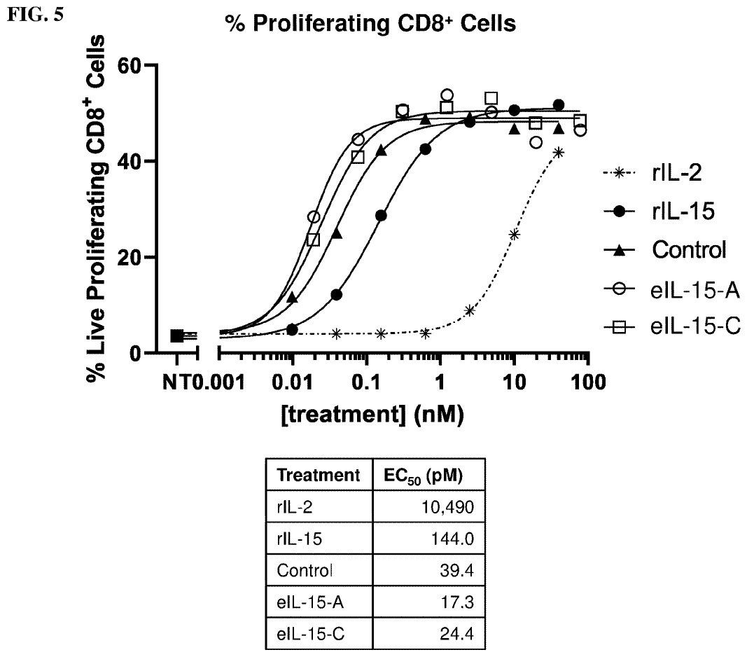

As shown in FIGS. 4A-4C, all mutant IL-15 complexes tested promoted similar or greater CD8+ T cell proliferation in vitro than control IL-15 complexes or recombinant IL-15, except eIL-15-B (FIG. 4A).

### Example 4: Flow Cytometric Analysis of Proliferation and Induction of CD25 and CD69 Expression

In this example, exemplary eIL-15 complexes were further tested for stimulation of primary human CD8⁺ T cell division and induction of T cell activation markers, CD25 and CD69, by flow cytometry.

Primary human CD8⁺ T cells (iQ Biosciences, Cat. No. IQB-Hu1-CD8T10, Lot P19K0200) were loaded with 1 μM CellTrace CFSE (Invitrogen, Cat. No. C34554, Lot 2208524). The cells were cultured at 1×10⁵ cells/well in 100 μL media/well (Immunocult-XF T Cell Expansion Medium, STEMCELL, Cat. No. 10981, Lot 1000072887) for three days, then washed and resuspended in 100 μL/well media alone or media treated with purified IL-15 complexes containing eIL-15-A or eIL-15-C variants or control (SEQ ID NO: 3; with or without a 6×His tag), recombinant human IL-15 (rIL-15) or recombinant human IL-2 (rIL-2) in a 7-point, 4-fold titration covering doses of 40 nM-9.8 pM, 79 nM-19.3 pM, or 78 nM-19 pM and cultured for an additional 4 days.

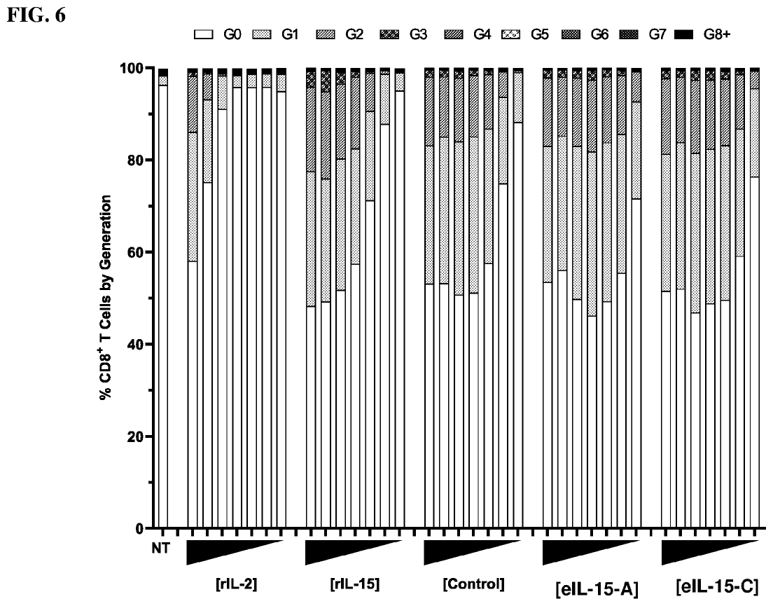

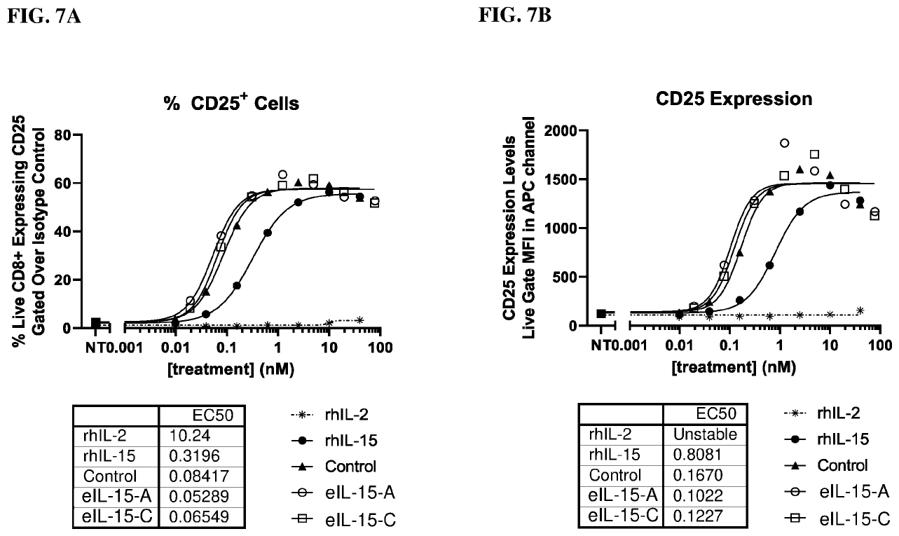

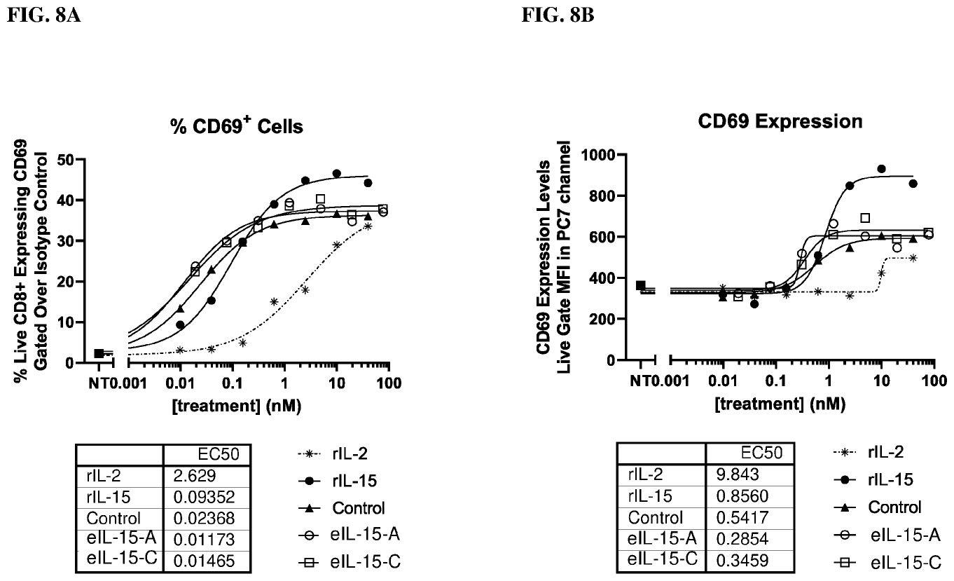

In preparation for flow cytometric assay, the cells were pelleted and resuspend in Zombie Violet Live/Dead in PBS for 30 minutes at room temperature. The cells were then washed and resuspended in FACS buffer (PBS+1% FBS+5 mM EDTA) containing anti-CD25 (clone M-A251)-AlexaFluor® 647 (ALX647) and anti-CD69-PE/Cy7 staining antibodies or isotype control antibodies for 30 minutes on ice, followed by a wash and resuspension in FACS buffer for acquisition. CFSE signal was measured in the FITC (525/40) channel, CD25 (ALX647) was measured in the APC (660/20) channel, CD69 (PE/Cy7) was measured in the PC7 (780/60) channel, and the Zombie Violet live/dead cells were measured in the PB450 (450/45) channel. Events were gated for whole, single, live cells. CD25⁺ and CD69⁺ events were gated over isotype control signal, and the CFSE signal was gated as “undivided” initial peak and subsequent generation peaks. Proliferation dose response curves were generated and EC₅₀ values were calculated (FIG. 5). The % CD8⁺ T cells in generations G₀ to ≥G₈ are shown in FIG. 6. The % of CD8⁺ T cells expressing CD25 and the levels of CD25 expression are presented in FIGS. 7A and 7B, respectively. The % of CD8⁺ T cells expressing CD69 and the CD69 expression levels are presented in FIGS. 8A and 8B, respectively.

Treatment with recombinant IL-15 and the IL-15 complexes stimulated much more proliferation of CD8⁺ T cells than treatment with recombinant IL-2 (FIG. 5 and FIG. 6). Of the IL-15 complexes tested, treatment with eIL-15-A or eIL-15-B resulted in more proliferation than the control IL-15 complex (FIG. 5). Generational analysis confirmed that the increased proliferation, indicated by increased percentage of cells in later generations, was concentration-dependent (FIG. 6).

Recombinant IL-15 and the IL-15 complexes induced expression of T cell activation markers CD25 (FIGS. 7A and 7B) and CD69 (FIGS. 8A and 8B), with eIL-15 complexes eIL-15-A and eIL-15-C inducing the highest levels of expression, particularly at lower concentrations. IL-2 did not induce expression of CD25 (FIGS. 7A and 7B) and effected lower expression of CD69.

### Example 5: Proliferation of Natural Killer (NK) Cells

The eIL-15 complexes were tested for their abilities to stimulate proliferation of natural killer (NK; i.e., CD56⁺) cells.

Human peripheral blood mononuclear cells (PBMCs) were isolated from leukocyte-enriched LSR chambers (San Diego Blood Bank) using Lymphopure™ (BioLegend, Cat. No. 426202) and SepMate™-50 (StemCell Technologies, Cat. No. 85450). The blood from an LSR chamber (8˜9 mL) was diluted with PBS to 20 mL and loaded onto a SepMate™ tube containing 15 mL of Lymphopure. The tube was centrifuged at 1,200×g for 10 minutes. The PBMC layer was transferred to a 50-mL conical tube containing 30 mL PBS and pelleted. The supernatant was discarded, and the cell pellet was washed with PBS. After spinning down and discarding the supernatant, 10⁸ cells were frozen in 1 mL fetal serum albumin containing 10% DMSO.

For cell proliferation assays, PBMCs were thawed in 10 mL RPMI 1640 medium containing 10% FBS and 1% penicillin/streptomycin. The cells were pelleted and resuspended in 2 mL PBS containing 5 mM CellTrace™ Violet (Thermo Fisher, Cat. No. C34571), then incubated at 37° C. for 20 minutes. The reaction was terminated by adding 10 mL RPMI culture medium. After 5 minutes, the cells were pelleted and resuspended in RPMI culture medium at 1×10⁶ cells/mL. After incubating 10 minutes at room temperature, 2×10⁵ cells in 200 μL were seeded in a 96-well plate. eIL-15-A, eIL-15-C, or control IL-15 (SEQ ID NO: 2) complexes or recombinant human IL-2 (rIL-2) cytokines were added into each well to yield final concentrations of 1.6 pM to 1 nM (rIL-2, Control), 3.2 pM to 2 nM (eIL-15-A), or 3.0 pM to 1.9 nM (eIL-15-C) in 5-fold dilutions. The cells were cultured for 4 days and replenished with 100 μL fresh medium for 3 additional days of culturing. The cells were then pelleted and resuspended in 50 μL FACS buffer (PBS, 2% FBS, and 2 mM EDTA) containing 1:10 diluted Human BD Fc Block (BD, Cat. No. 564220). After incubating on ice for 15 minutes, 50 μL of 1:10 diluted PE anti-human CD56 (NCAM) Antibody (BioLegend, Cat. No. 318306) were added. The cells were incubated on ice for 30 minutes and then 150 μL FACS buffer were added to the wells. After spinning down and removing supernatants, cells were resuspended in 150 μL FACS buffer and analyzed with a CytoFlex™ flow cytometer. NK proliferation % was defined by the percentage of CD56⁺ cells with reduced CFSE intensity.

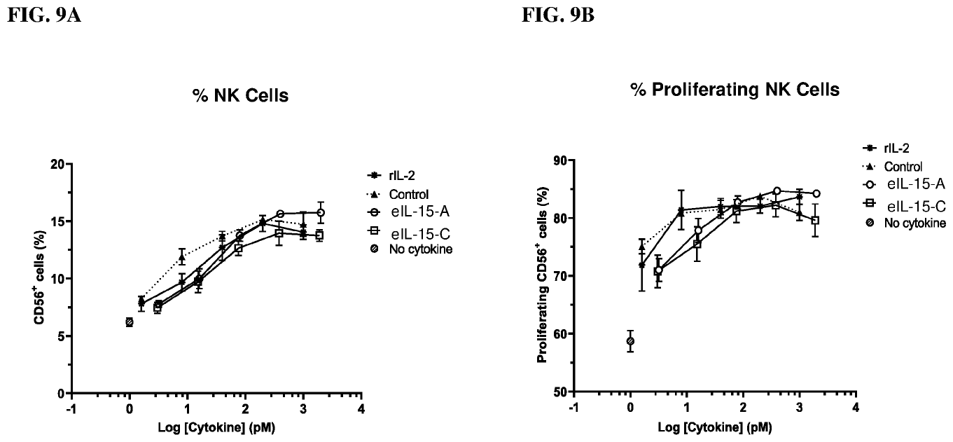

The % CD56⁺ cells and the percentage of proliferating CD56+cells were plotted as a function of cytokine concentration (FIGS. 9A and 9B, respectively). As shown in FIGS. 9A and 9B, treatment with all experimental and control IL-15 complexes, and recombinant IL-2, resulted in dose-dependent NK cell proliferation.

### Example 6: EIL-15-Induced Cytotoxic Activity of Human NK Cells

In this Example, eIL-15 complexes were tested for the ability to trigger cytotoxic activity of human NK cells toward the human leukemia cell line K-562.

Human NK cells were isolated from human PBMCs (San Diego Blood Bank) using an EasySep™ Human NK Cell Isolation kit (StemCell Technologies, Cat. No. 17955). Isolated NK cells were resuspended in complete RPMI 1640 culture medium (10% FBS, 1% penicillin/streptomycin) and 5×10⁵ cells in 100 μL culture medium were dispensed into each well of a U-bottom 96-well plate (Greiner, Cat. No. 650180). eIL-15 complexes containing eIL-15-A, eIL-15-C, control IL-15 complex, or recombinant human IL-2 were added to final concentrations of 0, 0.001, 0.01, 0.1, or 1 nM (Control or IL-2), 0.002, 0.02, 0.2, or 2 nM (eIL-15-A or eIL-15-C), and incubated at 37° C., 5% CO₂ for 20 hours.

To perform the cytotoxicity assay, human leukemia cell line K-562 (ATCC, Cat. No. CCL-243), which express a high level of NKG2D ligand, served as target cells. K-562 cells were resuspended in 2 mL PBS, labeled with 1 mM CellTrace™ Far Red (Thermo Fisher, Cat. No. C34564) and incubated at 37° C. for 20 minutes. Following incubation, 10 mL complete RPMI culture medium were added to terminate the reaction. After a 5-minute incubation, cells were pelleted then resuspended in complete RPMI culture medium at a concentration of 1×10⁶ cells/mL. Following further incubation for 10 minutes at room temperature, 1×10⁵ K-562 cells were transferred and mixed with the 5×10⁵ stimulated NK cells in each well for an effector cell to target cell ratio (E:T) of 5:1. The plates were centrifuged at 250×g for 2 minutes and co-cultured for 5 hours in the incubator. The plates were then centrifuged at 300×g for 5 minutes, the medium was removed, and the cells were resuspended in 200 μL FACS buffer containing 5 μL propidium iodide (BioLegend, Cat. No. 421301). Following a 10-minute incubation, the cells were analyzed by a CytoFlex™ flow cytometer. Dead target cells were identified as APC⁺/PE⁺. Specific cytotoxicity was calculated by dead target cell percentages of cytokine-treated groups subtracted from the dead cell percentage of the control group without cytokine treatment.

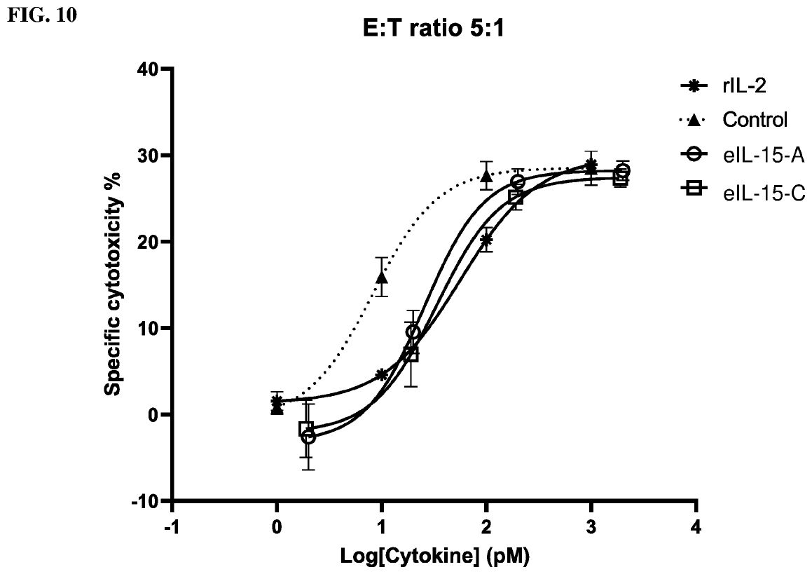

As shown in FIG. 10, NK cell treatment with all of the tested eIL-15 complexes and the control IL-15 complex resulted in potent NK cell cytotoxic activity against K-562 target cells.

### Example 7: Effect of Engineered IL-15 on NK Cell-Mediated Antibody-Dependent Cellular Cytotoxicity (ADCC) Activity

In this Example, the exemplary engineered IL-15 molecule, eIL-15-C, was tested for enhancement of NK cell-mediated ADCC activity of antibody-bound target cells.

Human NK cells were isolated from human PBMCs (San Diego Blood Bank) as described in the previous Example. Isolated NK cells were resuspended in complete RPMI 1640 culture medium and 1×10⁵ cells in 100 μL culture medium were dispensed into each well of a U-bottom 96-well plate (Greiner, Cat. No. 650180), except two “No NK” control wells. Engineered IL-15 complex, eIL-15-C, or recombinant human IL-2 were added to the plated NK cells to achieve final concentrations of 0, 0.1, 1.0, 10, 100, or 1000 pM (rIL-2) or 0.19, 1.9, 19, 190, or 1900 pM (eIL-15-C complex). The cells and cytokines were incubated at 37° C. for 20 hours.

Cells of the human epithelial squamous cell carcinoma cell line Cal 27 (ATCC, Cat. No. CCL-2095) served as target cells to measure NK cytotoxic activity. Cal 27 cells were resuspended in 2 mL PBS, labeled with 1 mM CellTrace™ Far Red (Thermo Fisher, Cat. No. C34564) and incubated at 37° C. for 20 minutes. Following incubation, 10 mL complete DMEM culture medium (DMEM, 10% FBS, 1% penicillin/streptomycin) were added to terminate the reaction. After 5 minutes, the labeled cells were spun down, the medium was removed, and the cells were resuspended in DMEM culture medium at a concentration of 1×10⁶ cells/mL. Cetuximab (BioXCell, Cat. No. SIM0002) was added to a final concentration of 1 nM and incubated at room temperature for 20 minutes. 100 μL (1×10⁵) labeled Cal 27 cells were transferred and gently mixed with the cytokine-treated NK cells in the 96-well plate for an effector (NK) cell to target (Cal 27) cell ratio of 1:1. The cells were spun down at 250×g for 2 minutes and incubated for 5 hours 37° C. The cells were then spun down at 300×g for 5 minutes, the medium was removed, and 50 μL Accutase (Thermo Fisher, Cat. No. 00-4555-56) were added. The cells were incubated at 37° C. for 20 minutes to detach the Cal 27 cells. The cell mixture was then resuspended in 150 μL FACS buffer containing 5 L propidium iodide (BioLegend, cat #421301). After mixing, the cells were incubated at room temperature for 10 minutes and then analyzed by a CytoFlex™ flow cytometer. Dead target cells were identified as APC⁺/PE⁺.

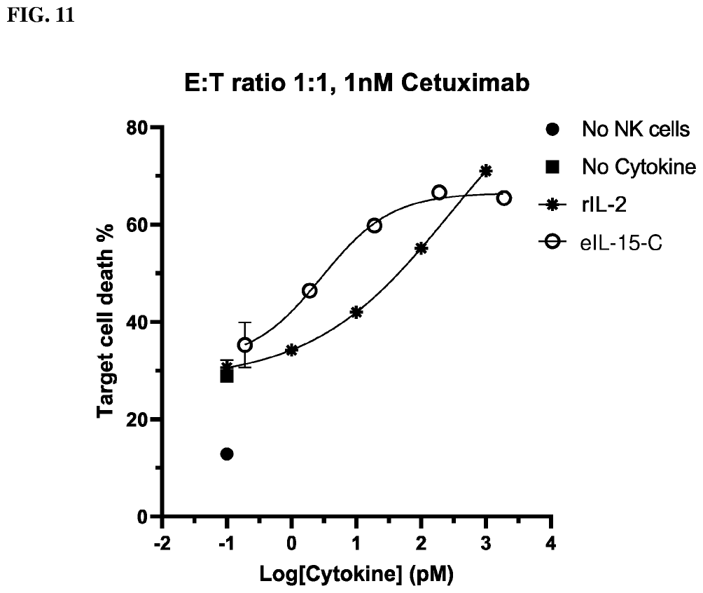

As shown in FIG. 11, little cell death was observed for labeled Cal 27 cells in the absence of NK cells (filled circle). Incubating NK cells with eIL-15-C resulted in a dose-dependent increase in NK-mediated killing of antibody-bound target cells (open circles). Incubating NK cells with rIL-2 also resulted in a dose-dependent increase in NK-mediated killing of target cells, but to a lesser extent (asterisks). These results support a synergistic role of eIL-15-C in enhancing ADCC activity of NK cells.

### Example 8: IL-15 Treatment in Combination with Cancer-Targeted Photoimmunotherapy (PIT) In Viro

LL/2 murine lung carcinoma cells were engineered to express the murine antigen Ephrin type-A receptor 2 (EphA2) to generate an LL/2-EphA2 cell line. Mice develop tumors when injected with LL/2-EphA2 cells, yielding a mouse tumor model. A conjugate containing an antibody that specifically binds to EphA2 and IRDye 700Dx (anti-EphA2-IR700) has been described previously (Hsu et al. Cancer Immunol Immunother. 2022 Jul. 1 doi: 10.1007/s00262-022-03239-9). Illumination of the anti-EphA2-IR700 conjugate at near infrared wavelengths activates the IR700 dye and results in tumor killing, resulting in cancer-targeted photoimmunotherapy (PIT). The following study was performed to assess whether treatment with the eIL-15 complexes described herein enhance the cancer cell killing activity of cancer-targeted PIT.

C57B1/6 mice (6-8 weeks of age) were inoculated with 5×10⁵ LL/2-EphA2 cells subcutaneously in the right hind flank. When allograft tumors grew to about 125 mm³ (approximately 6 days after implantation), the mice were divided into the following treatment groups, each containing 10 mice: (1) saline only, (2) cancer-targeted EphA2 PIT (PIT), (3) eIL-15-A (SEQ ID NO: 30) complex monotherapy, (4) eIL-15-A+PIT, (5) eIL-15-C(SEQ ID NO: 32) complex monotherapy, (6) eIL-15-C+PIT. On Day 6, the mice in the saline group were administered saline (100 μL), and mice in the PIT or PIT combination groups were administered EphA2-IR700 conjugate (100 μg) via retroorbital injection for PIT. On Day 7, mice administered the conjugate were illuminated at 690 nm, at a dose of 200 J/cm². Animals receiving eIL-15 treatment were administered 8 pg eIL-15-A complex or eIL-15-C complex on Days 7 and 10, followed by every 4 days for 3 weeks. The tumor growth and survival were measured until Day 22-36. Tumor volume was calculated using the formula: tumor volume=(width×length)×height/2 after measuring with calipers.

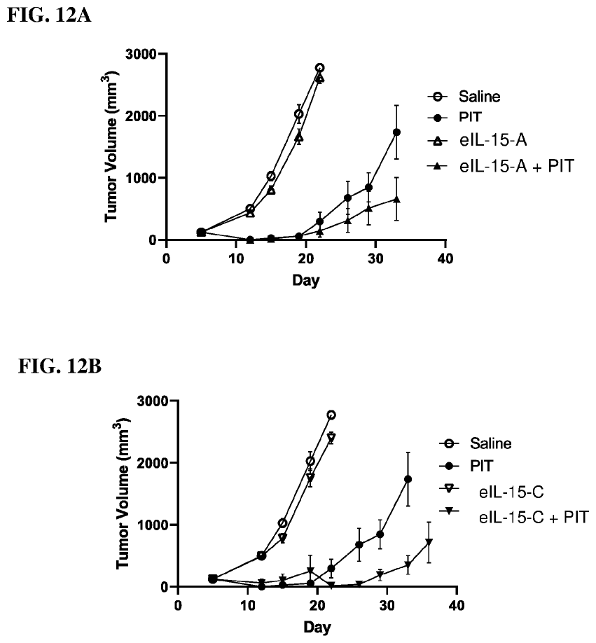

Tumor growth in mice treated with eIL-15-A complex or eIL-15-C complex, as a monotherapy or in combination with PIT, compared to saline or PIT monotherapy is shown in FIGS. 12A and 12B, respectively. All eIL-15 complex+PIT combination treatments significantly decreased tumor growth compared to the individual monotherapies.

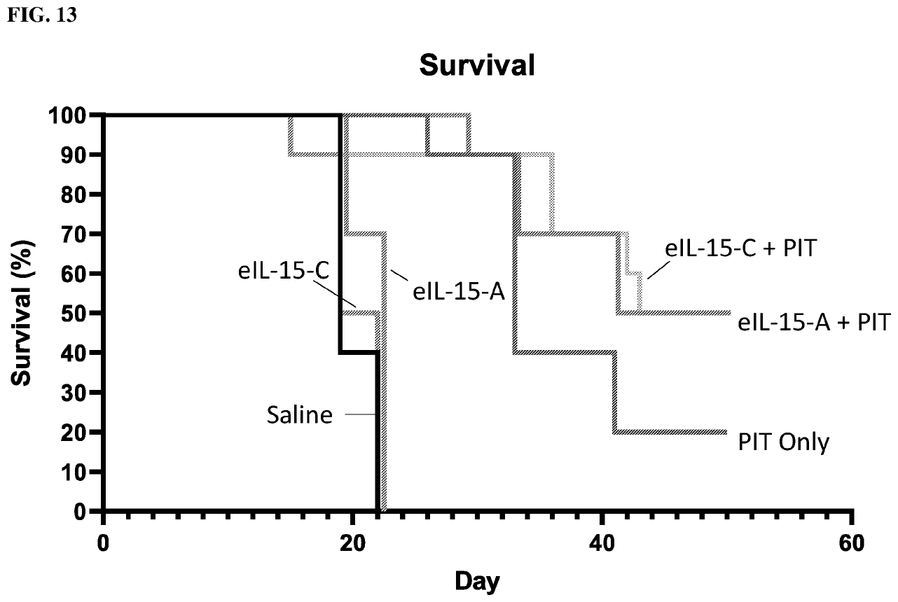

As shown in FIG. 13, survival of mice treated with saline or eIL-15-A complex or eIL-15-C complex alone rapidly declined around Day 20. Animals treated with PIT monotherapy exhibited improved survival compared to saline or eIL-15 monotherapies. Animals treated with the combination of eIL-15-A complex+PIT or eIL-15-C complex+PIT exhibited the best survival. The survival results are consistent with the tumor growth inhibition observed in FIGS. 12A-12B.

These results evidence synergy between eIL-15 treatment and PIT by demonstrating improved activity for the eIL-15 complexes in combination with PIT compared to the individual monotherapies.

### Example 9: Effect of Engineered IL-15 on Systemic Immune Cell Populations in Tumor-Bearing Mice

CT26 murine colon carcinoma (3×10⁶) were implanted into immunocompetent BALB/c mice to generate a mouse tumor model. Ten (10) days after tumor seeding, mice were administered 2 μg eIL-15-C(SEQ ID NO: 32) complex (n=10 mice), 4 μg control IL-15 (SEQ ID NO: 3) complex (n=10 mice), or saline (n=10 mice) by intraperitoneal injection. Three (3) days later mice were sacrificed, and spleens and blood were harvested from each mouse.

Individual spleens were transferred to a 70 μm strainer, pre-wetted with 3 mL RPMI-1640 cell culture media and placed over a 15 mL conical tube. The spleens were then mashed with the plunger of a 10 mL syringe, washed with an additional 9 mL of RPMI-1640 and placed on ice until all spleens were processed. The tissue homogenates were then centrifuged, red blood cells lysed, and remaining cells (splenocytes) washed with PBS. Splenocytes were counted on the Vi-Cell Blu automated cell counter. 1×10⁶ cells were transferred to the appropriate wells and surface stained, fixed, permeabilized, and intracellularly stained. Cells were resuspended in PBS, an equal volume of Precision Counting Beads were added, and cells were analyzed for total T (CD3+) cell, cytotoxic T (CD3+, CD8+) cell, Helper T (CD3+, CD4+) cell, NK cell (CD3−, CD49b+), NK-T (CD49b+, CD3+) cell, and regulatory T (FOXP3+, CD4+, CD3+) cell (Tregs) content by flow cytometry on a CytoFLEX (Beckman Coulter) flow cytometer.

Blood samples were processed by transferring 100 μL of blood from each mouse to corresponding 15 mL conical tubes containing surface stain antibodies and incubated for 15 minutes at room temperature. Red blood cells were lysed with 2 mL 1× RBC Lysis Buffer for 15 minutes. Remaining cells were washed, centrifuged, decanted, and resuspended in residual volume. Residual volume was transferred to the appropriate well of a 96-well plate, washed, centrifuged, fixed, permeabilized, and intracellularly stained. Cells were resuspended in PBS, an equal volume of Precision Counting Beads were added, and cells were analyzed for total T (CD3+) cell, cytotoxic T (CD3+, CD8+) cell, Helper T (CD3+, CD4+) cell, NK cell (CD3−, CD49b+), NK-T (CD49b+, CD3+) cell, and regulatory T (FOXP3+, CD4+, CD3+) cell (Tregs) content by flow cytometry on a CytoFLEX (Beckman Coulter) flow cytometer.

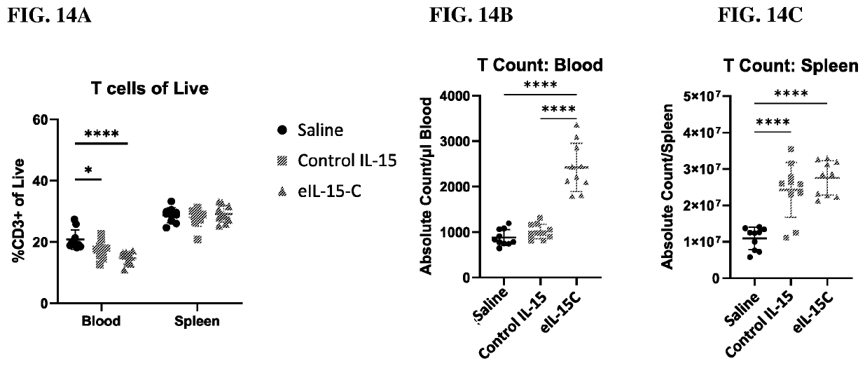

As shown in FIG. 14A, the peripheral T cells (in blood) expanded less compared to other cell populations as indicated by the decreased frequency of total live cells in tumor-bearing animals treated with IL-15 or eIL-15 complexes compared to untreated (saline). This difference was even more significant for the eIL-15-treated animals. As shown in FIG. 14B, the peripheral T cell count, however, was highest in animals treated with eIL-15 complex. In spleen, tumor-bearing animals treated with eIL-15 complex and control IL-15 complex exhibited higher levels of T cell counts compared to saline treatment, as shown in FIG. 14C. There was an approximately 2.5-fold increase in total T cell count in mice treated with eIL-15-C compared to saline in peripheral blood and spleen (FIGS. 14B and 14C).

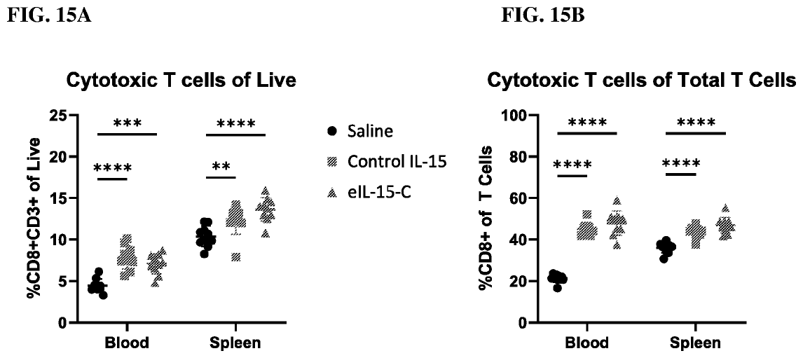

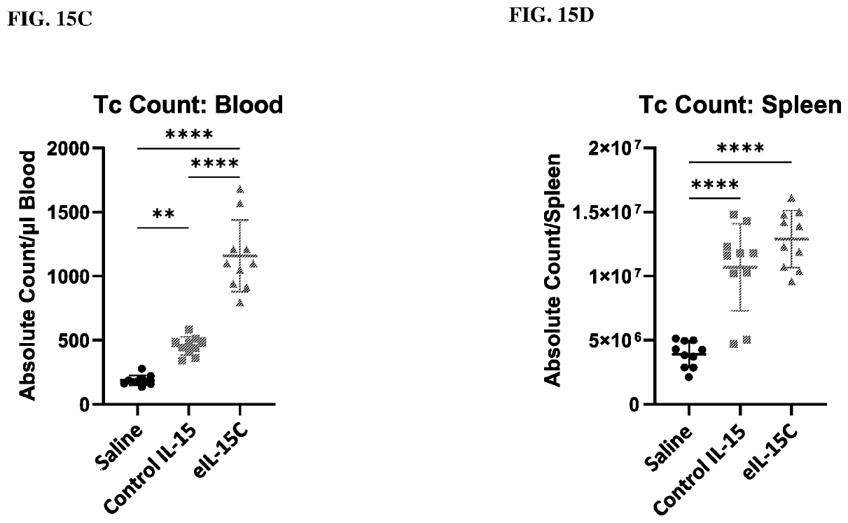

As shown in FIG. 15A treatment with eIL-15-C complex or control IL-15 complex resulted in an increased frequency of cytotoxic (CD8+) T cells in the blood and spleen of tumor-bearing mice. The frequency of cytotoxic (CD8+) T cells among all T cells was also increased in blood and spleen in eIL-15 complex and control IL-15 complex-treated animals (FIG. 15B). The number of cytotoxic (CD8+) T cells in the blood and spleen were increased in the blood and spleens of mice treated with eIL-15-C or the control complex, with a greater increase observed in mice treated with eIL-15C (FIG. 15C and FIG. 15D, respectively).

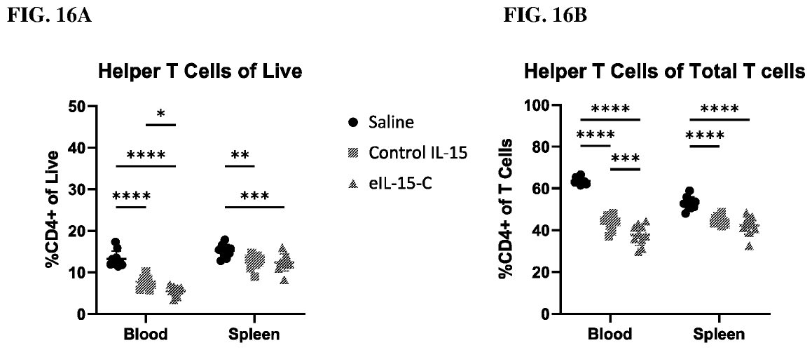

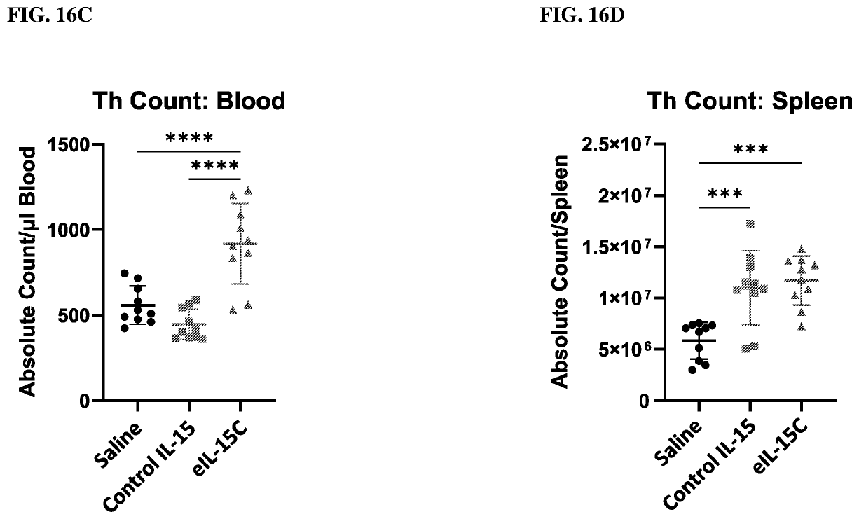

As shown in FIG. 16A and FIG. 16B, treatment with eIL-15-C complex or control IL-15 complex resulted in a decrease in the frequency of helper (CD3+, CD4+) T cells as a percentage of total live cells (FIG. 16A) or as a percentage of total T cells (FIG. 16B) compared to saline in blood and spleen samples collected from tumor-bearing mice. The percentage of helper (CD3+, CD4+) T cells in the blood of animals treated with eIL-15-C complex was significantly lower than either of the other treatment groups. In terms of total cell counts, tumor-bearing mice treated with eIL-15-C complex had a 1.5-2-fold increase in the number of helper (CD3+, CD4+) T cells in the blood and spleen compared to saline controls (FIG. 16C and FIG. 16D).

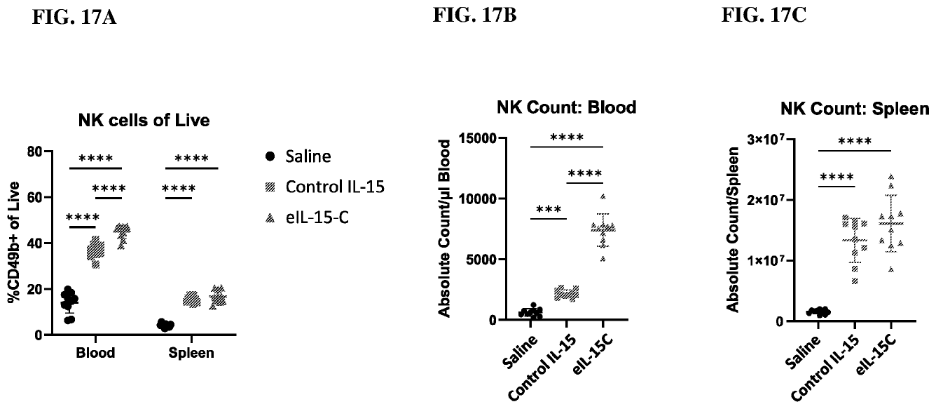

As shown in FIG. 17A, the percentage of NK (CD3−, CD49b+) cells in the blood and spleen increased in tumor-bearing mice treated with eIL-15-C complex or control IL-15 complex, with mice having received treatment with eIL-15-C complex exhibiting a significantly higher percentage NK cells in the blood than the other treatment groups (FIG. 17A). Compared to saline controls, counts of NK cells in the blood and spleen were higher in mice treated with the eIL-15-C complex (approximately 10-fold higher) or control IL-15 complex (FIG. 17B and FIG. 17C). There were significantly more NK cells counted in the blood following treatment with eIL-15-C complex than treatment with control IL-15 complex (FIG. 17B).

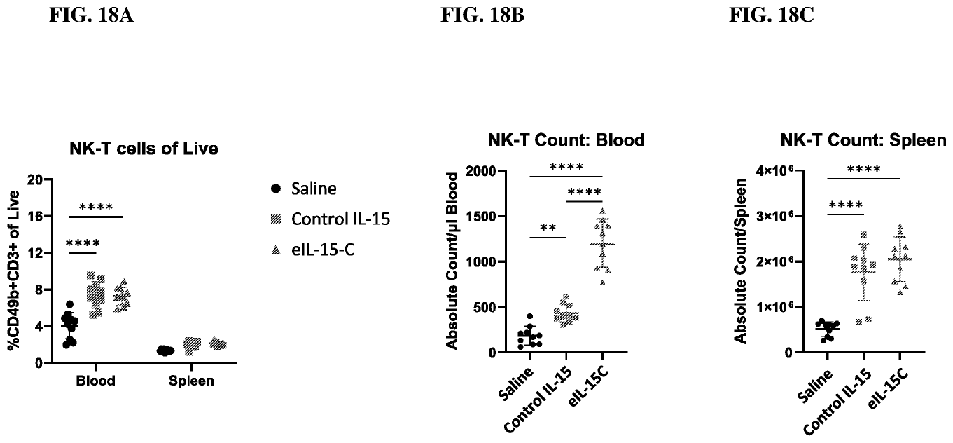

As shown in FIG. 18A, the percentage of NK-T (CD49b+, CD3+) cells was increased in the blood of mice treated with eIL-15-C complex or control IL-15 complex compared to saline. This increase corresponded with an increase in the count of NK-T cells in the blood following eIL-15-C complex or control IL-15 complex treatment (FIG. 18B), with a greater increase in NK-T cells following treatment with eIL-15-C complex than following control IL-15 complex treatment. While there was no significant difference in the percent of NK-T cells in the spleen following either IL-15 complex treatment, the count of NK-T cells in the spleen of mice treated with eIL-15-C complex or control IL-15 complex was increased compared to saline controls (FIG. 18C). These results indicate that NK-T cells are sensitive to stimulation by eIL-15-C complex.

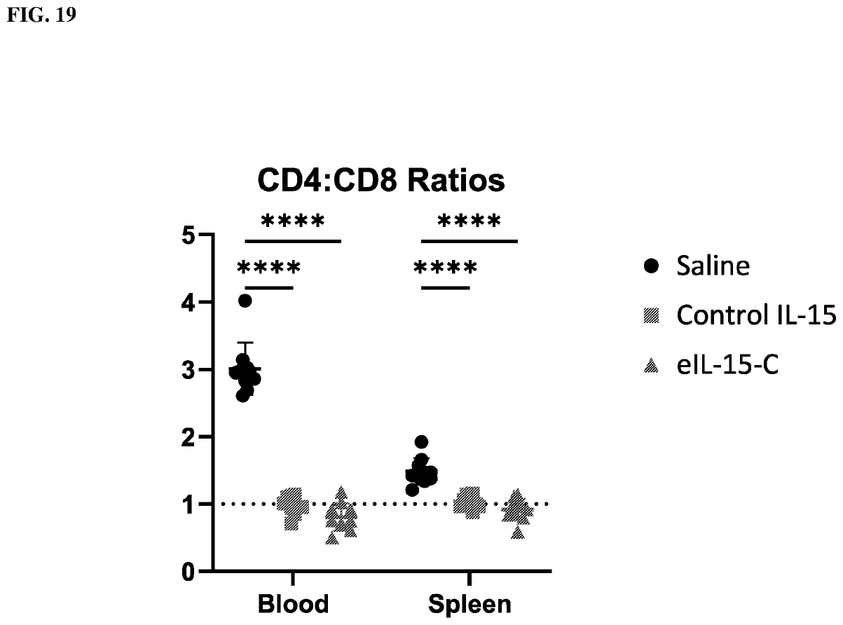

Further, CD4:CD8 ratios were reduced in the blood and spleens of animals treated with eIL-15-C complex or control IL-15 complex (FIG. 19).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID | | |
|---|---|---|
| NO. | Sequence | Description |
| 1 | MRISKPHLRSISIQCYLCLLLNSHFLTEAGIHVFILGCFSAGLPKTEA | hIL-15 (UniProt |
| | NWVNVISDLKKIEDLIQSMHIDATLYTESDVHPSCKVTAMKCFLLELQ | P40933) |
| | VISLESGDASIHDTVENLIILANNSLSSNGNVTESGCKECEELEEKNI | |
| | KEFLQSFVHIVQMFINTS | |
| 2 | NWVNVISDLKKIEDLIQSMHIDATLYTESDVHPSCKVTAMKCFLLELQ | mature hIL-15 |
| | VISLESGDASIHDTVENLIILANNSLSSNGNVTESGCKECEELEEKNI | |
| | KEFLQSFVHIVQMFINTS | |
| 3 | NWVNVISDLKKIEDLIQSMHIDATLYTESDVHPSCKVTAMKCFLLELQ | Control IL-15 |
| | VISLESGDASIHDTVENLIILANDSLSSNGNVTESGCKECEELEEKNI | |
| | KEFLQSFVHIVQMFINTS | |
| 4 | NWINVISDLKKIEDLIQSMHIDATLYTESDVHPSCKVTAMKCFLLELQ | V3I |
| | VISLESGDASIHDTVENLIILANNSLSSNGNVTESGCKECEELEEKNI | |
| | KEFLQSFVHIVQMFINTS | |
| 5 | NWWNVISDLKKIEDLIQSMHIDATLYTESDVHPSCKVTAMKCFLLELQ | V3W |
| | VISLESGDASIHDTVENLIILANNSLSSNGNVTESGCKECEELEEKNI | |
| | KEFLQSFVHIVQMFINTS | |
| 6 | NWVHVISDLKKIEDLIQSMHIDATLYTESDVHPSCKVTAMKCFLLELQ | N4H |
| | VISLESGDASIHDTVENLIILANNSLSSNGNVTESGCKECEELEEKNI | |
| | KEFLQSFVHIVQMFINTS | |
| 7 | NWVNVLSDLKKIEDLIQSMHIDATLYTESDVHPSCKVTAMKCFLLELQ | I6L |
| | VISLESGDASIHDTVENLIILANNSLSSNGNVTESGCKECEELEEKNI | |
| | KEFLQSFVHIVQMFINTS | |
| 8 | NWVNVISDLRKIEDLIQSMHIDATLYTESDVHPSCKVTAMKCFLLELQ | K10R |
| | VISLESGDASIHDTVENLIILANNSLSSNGNVTESGCKECEELEEKNI | |
| | KEFLQSFVHIVQMFINTS | |
| 9 | NWVNVISDLYKIEDLIQSMHIDATLYTESDVHPSCKVTAMKCFLLELQ | K10Y |
| | VISLESGDASIHDTVENLIILANNSLSSNGNVTESGCKECEELEEKNI | |
| | KEFLQSFVHIVQMFINTS | |
| 10 | NWVNVISDLKEIEDLIQSMHIDATLYTESDVHPSCKVTAMKCFLLELQ | K11E |
| | VISLESGDASIHDTVENLIILANNSLSSNGNVTESGCKECEELEEKNI | |
| | KEFLQSFVHIVQMFINTS | |
| 11 | NWVNVISDLKYIEDLIQSMHIDATLYTESDVHPSCKVTAMKCFLLELQ | K11Y |
| | VISLESGDASIHDTVENLIILANNSLSSNGNVTESGCKECEELEEKNI | |
| | KEFLQSFVHIVQMFINTS | |
| 12 | NWVNVISDLKKIEDLIQSMHIDPTLYTESDVHPSCKVTAMKCFLLELQ | A23P |
| | VISLESGDASIHDTVENLIILANNSLSSNGNVTESGCKECEELEEKNI | |
| | KEFLQSFVHIVQMFINTS | |
| 13 | NWVNVISDLKKIEDLIQSMHIDAPLYTESDVHPSCKVTAMKCFLLELQ | T24P |
| | VISLESGDASIHDTVENLIILANNSLSSNGNVTESGCKECEELEEKNI | |
| | KEFLQSFVHIVQMFINTS | |
| 14 | NWVNVISDLKKIEDLIQSMHIDATLYTESDVHPSCKVTAMKCFLIELQ | L45I |
| | VISLESGDASIHDTVENLIILANNSLSSNGNVTESGCKECEELEEKNI | |
| | KEFLQSFVHIVQMFINTS | |
| 15 | NWVNVISDLKKIEDLIQSMHIDATLYTESDVHPSCKVTAMKCFLLRLQ | E46R |
| | VISLESGDASIHDTVENLIILANNSLSSNGNVTESGCKECEELEEKNI | |
| | KEFLQSFVHIVQMFINTS | |
| 16 | NWVNVISDLKKIEDLIQSMHIDATLYTESDVHPSCKVTAMKCFLLELQ | V49W |
| | WISLESGDASIHDTVENLIILANNSLSSNGNVTESGCKECEELEEKNI | |
| | KEFLQSFVHIVQMFINTS | |
| 17 | NWVNVISDLKKIEDLIQSMHIDATLYTESDVHPSCKVTAMKCFLLELQ | A57P |
| | VISLESGDPSIHDTVENLIILANNSLSSNGNVTESGCKECEELEEKNI | |
| | KEFLQSFVHIVQMFINTS | |
| 18 | NWVNVISDLKKIEDLIQSMHIDATLYTESDVHPSCKVTAMKCFLLELQ | S58P |
| | VISLESGDAPIHDTVENLIILANNSLSSNGNVTESGCKECEELEEKNI | |
| | KEFLQSFVHIVQMFINTS | |
| 19 | NWVNVISDLKKIEDLIQSMHIDATLYTESDVHPSCKVTAMKCFLLELQ | N72T |
| | VISLESGDASIHDTVENLIILANTSLSSNGNVTESGCKECEELEEKNI | |
| | KEFLQSFVHIVQMFINTS | |
| 20 | NWVNVISDLKKIEDLIQSMHIDATLYTESDVHPSCKVTAMKCFLLELQ | H105D |
| | VISLESGDASIHDTVENLIILANNSLSSNGNVTESGCKECEELEEKNI | |
| | KEFLQSFVDIVQMFINTS | |
| 21 | NWVNVISDLKKIEDLIQSMHIDATLYTESDVHPSCKVTAMKCFLLELQ | H105E |
| | VISLESGDASIHDTVENLIILANNSLSSNGNVTESGCKECEELEEKNI | |
| | KEFLQSFVEIVQMFINTS | |
| 22 | NWVNVISDLKKIEDLIQSMHIDATLYTESDVHPSCKVTAMKCFLLELQ | H105W |
| | VISLESGDASIHDTVENLIILANNSLSSNGNVTESGCKECEELEEKNI | |
| | KEFLQSFVWIVQMFINTS | |
| 23 | NWVNVISDLKKIEDLIQSMHIDATLYTESDVHPSCKVTAMKCFLLELQ | N112F |
| | VISLESGDASIHDTVENLIILANNSLSSNGNVTESGCKECEELEEKNI | |
| | KEFLQSFVHIVQMFIFTS | |
| 24 | NWVNVISDLKKIEDLIQSMHIDATLYTESDVHPSCKVTAMKCFLLELQ | N112L |
| | VISLESGDASIHDTVENLIILANNSLSSNGNVTESGCKECEELEEKNI | |
| | KEFLQSFVHIVQMFILTS | |
| 25 | NWVNVISDLKKIEDLIQSMHIDATLYTESDVHPSCKVTAMKCFLLELQ | N112Y |
| | VISLESGDASIHDTVENLIILANNSLSSNGNVTESGCKECEELEEKNI | |
| | KEFLQSFVHIVQMFIYTS | |
| 26 | NWVNVISDLKKICDLIQSMHIDATLYTESDVHPSCKVTAMKCFLLELQ | E13C/L100C |
| | VISLESGDASIHDTVENLIILANNSLSSNGNVTESGCKECEELEEKNI | |
| | KEFCQSFVHIVQMFINTS | |
| 27 | NWVNVISDLKKIEDCIQSMHIDATLYTESDVHPSCKVTAMKCFLLELQ | L15C/I59C |
| | VISLESGDASCHDTVENLIILANNSLSSNGNVTESGCKECEELEEKNI | |
| | KEFLQSFVHIVQMFINTS | |
| 28 | NWVNVISDLKKIEDLIQSMHIDACLYTESDVHPSCKVTAMKCFLLELQ | T24C/E93C |
| | VISLESGDASIHDTVENLIILANNSLSSNGNVTESGCKECEELECKNI | |
| | KEFLQSFVHIVQMFINTS | |
| 29 | NWVNVISDLKKIEDLIQSMHIDATLYTECDVHPSCKVTAMKCFLLELQ | S29C/S102C |
| | VISLESGDASIHDTVENLIILANNSLSSNGNVTESGCKECEELEEKNI | |
| | KEFLQCFVHIVQMFINTS | |
| 30 | NWVNVISDLRYIESLISSMNIDACLYTESDVHPKCSVTAMLCFLLELQ | eIL-15-A |
| | VISLESGDPSIHDTVENLIILANNSLSSNGNKTEDGCKECEELECKNI | |
| | KEFLQSFVHIVQMFINTS | |
| 31 | NWVNVISDLRYIEDCISNMHIDAPLYTESDVHPKCSVTAMLCFLLELQ | eIL-15-B |
| | VISLESGDPSCHDTVENLIILANNSLSSNGNVTESGCKECEELEEKNI | |
| | KEFLQSFVWIVQMFILTS | |
| 32 | NWVNVISDLREIESLIQNMHIDALLYTECDVHPKCKVTAMKCFLLELQ | eIL-15-C |
| | VISLESGDAPIHDTVENLIILANNSLSSNGNKTEDGCKECEELEEKNI | |
| | KEFLQCFVWIVQMFILTS | |
| 33 | NWVNVISDLREIESLISSMNIDACLYTESDVHPKCSVTAMKCFLLELQ | eIL-15-D |
| | VISLESGDASIHDTVENLIILANNSLSSNGNKTEDGCKECEELECKNI | |
| | AEFLQSFVHIVQMFINTS | |
| 34 | NWVNVISDLREIESLISSMNIDACLYTESDVHPKCSVTAMKCFLLELQ | eIL-15-E |
| | VISLESGDASIHDTVENLIILANNSLSSNGNKTEDGCKECEELECKNI | |
| | KEFLQSFVWIVQMFINTS | |
| 35 | NWVNVISDLREIESLISSMHIDACLYTESDVHPKCSVTAMKCFLLELQ | eIL-15-F |
| | VISLESGDASIHDTVENLIILANNSLSSNGNKTEDGCKECEELECKNI | |
| | AEFLQSFVWIVQMFINTS | |
| 36 | NWVNVISDLREIENLISSMNIDACLYTESDVHPSCKVTAMKCFLLELQ | eIL-15-G |
| | VISLESGDAQIHDTVENLIILANNSLSSNGNYTEDGCKECEELECKNI | |
| | AEFLQSFVWIVQMFINTS | |
| 37 | NWVNVISDLREIENLIQSMNIDACLYTESDVHPKCSVTAMKCFLLELQ | eIL-15-H |
| | VISLESGDASIHDTVENLIILANNSLSSNGNKTEDGCKECEELECKNI | |
| | AEFLQSFVWIVQMFINTS | |
| 38 | NWVNVISDLRYIESLISSMNIDACLYTESDVHPSCKVTAMKCFLLELQ | eIL-15-I |
| | VISRESGDASIHDTVENLIILANNSLSSNGNKTEDGCKECEELECKNI | |
| | AEFLQSFVHIVQMFILTS | |
| 39 | NWVDVISDLREIENLISSMHIDACLYTESDVNPRCSVTAMKCFLLELQ | eIL-15-J |
| | VISLESGDASIHDTVENLIILANNSLSSSGNVTESGCKECEELECKNI | |
| | KEFLQSFVHIVQMFANTS | |
| 40 | NWVEVISDLREIENLISSMHIDACLYTESDVSPRCSVTAMKCFLLELQ | eIL-15-K |
| | VISLESGDASIHDTVENLIILANNSLSSSGNVTESGCKECEELECKNI | |
| | KEFLQSFVHIVQMFANTS | |
| 41 | ITCPPPMSVEHADIWVKSYSLYSRERYICNSGFKRKAGTSSLTECVLN | IL-15Ralpha-Fc |
| | KATNVAHWTTPSLKCIRDGGGSGGGSEPKSSDKTHTCPPCPAPELLGG | |
| | PSVFLFPPKPKDILMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |

1. An engineered IL-15 polypeptide comprising at least 6 cysteine residues and capable of forming at least 3 intramolecular disulfide bonds.

2. The engineered IL-15 polypeptide of claim 1, wherein the IL-15 polypeptide sequence is derived from a mammalian IL-15.

3. The engineered IL-15 polypeptide of any one of claim 1, wherein the IL-15 polypeptide sequence is derived from a human IL-15.

4. The engineered IL-15 polypeptide of claim 1, comprising two amino acid substitutions in SEQ ID NO: 2, wherein the two amino acid substitutions are substituting a non-cysteine residue with a cysteine.

5. The engineered IL-15 polypeptide of any one of claims 1-4, wherein two of the cysteine residues are present at a position corresponding to positions 24 and 93 of SEQ ID NO:2 or corresponding to positions 29 and 102 of SEQ ID NO:2.

6. The engineered IL-15 polypeptide of any one of claims 1-5, comprising at least one amino acid substitution at a position corresponding to position 4, 10, 11, 14, 17, 18, 20, 24, 29, 32, 34, 36, 41, 52, 57, 58, 77, 80, 83, 93, 97, 102, 105, 111, or 112 of SEQ ID NO: 2.

7. The engineered IL-15 polypeptide of any one of claims 1-6, wherein the engineered IL-15 polypeptide comprises up to 2, 3, 4, 5, 6, 7, 8, 9, 10, 11, 12, 13, 14, 15, 16, 17, 18, 19 or 20 amino acid substitutions as compared to SEQ ID NO:2.

8. The engineered IL-15 polypeptide of any one of claims 1-7, wherein the engineered IL-15 polypeptide comprises one or more amino acid substitutions selected from the group consisting of N4D, N4E, K10R, K11Y, K11E, Q17S, S18N, H20N, T24C, T24L, S29C, H32N, H32S, S34K, S34R, K36S, K41L, L52R, A57P, S58P, S58Q, N77S, V80K, S83D, E93C, K97A, S102C, H105W, I111A, and N112L, with reference to positions of SEQ ID NO:2.

9. The engineered IL-15 polypeptide of claim 8, comprising 2, 3, 4, 5, 6, 7, 8, 9, 10, 11, 12, 13, 14, or 15 amino acid substitutions selected from the group consisting of N4D, N4E, K10R, K11Y, K11E, Q17S, S18N, H20N, T24C, T24L, S29C, H32N, H32S, S34K, S34R, K36S, K41L, L52R, A57P, S58P, S58Q, N77S, V80K, S83D, E93C, K97A, S102C, H105W, I111A, and N112L, with reference to positions of SEQ ID NO:2.

10. The engineered IL-15 polypeptide of any one of claims 1-9, wherein the engineered IL-15 polypeptide further comprises the amino acid substitution N72D, with reference to positions of SEQ ID NO:2.

11. The engineered IL-15 polypeptide of any one of claims 1-10, wherein the engineered IL-15 polypeptide has at least 75% identity and less than 90% identity to SEQ ID NO:2.

12. The engineered IL-15 polypeptide of any one of claims 1-5, wherein the engineered IL-15 polypeptide comprises one or more amino acid substitution in helix A, helix B, helix C, helix D of IL-15, or any combination thereof.

13. The engineered IL-15 polypeptide of any one of claims 1-5, wherein the engineered IL-15 polypeptide comprises one or more amino acid substitution in the loop region between helix A and helix B, between helix B and helix C, between helix C and helix D of IL-15, or any combination thereof.

14. The engineered IL-15 polypeptide of any one of claims 1-5, wherein the engineered IL-15 polypeptide comprises the addition of a cysteine in the loop region between helix A and helix B and/or between helix C and helix D of IL-15.

15. The engineered IL-15 polypeptide of claim 14, wherein the addition of the cysteine comprises a substitution of a cysteine for another amino acid in SEQ ID NO:2.

16. A complex, comprising the engineered IL-15 polypeptide of any one of claims 1-15 and a second polypeptide.

17. The complex of claim 16, wherein the second polypeptide comprises an antibody or antigen-binding fragment.

18. The complex of claim 16, wherein the second polypeptide comprises an Fc domain or a portion thereof.

19. The complex of claim 16, wherein the second polypeptide comprises a receptor molecule or domain thereof.

20. The complex of claim 19, wherein the second polypeptide comprises an IL-15 receptor molecule or domain thereof.

21. The complex of claim 19, wherein the second polypeptide comprises an IL-15 receptor sushi domain.

22. The complex of claim 16, wherein the second polypeptide comprises a receptor molecule or domain thereof fused to an Fc domain or a portion thereof.

23. The complex of claim 22, wherein the second polypeptide comprises an IL-15 receptor molecule or domain thereof fused to an Fc domain or a portion thereof.

24. The complex of claim 22 or 23, wherein the second polypeptide comprises an IL-15 sushi domain fused to an Fc domain.

25. A complex, comprising the engineered IL-15 polypeptide of any one of claims 1-15 and a second polypeptide comprising an IL-15 sushi domain fused to an Fc domain.

26. The complex of claim 24 or 25, wherein the second polypeptide comprises the amino acid sequence set forth in SEQ ID NO:41, or an amino acid sequence that has at least 90%, 91%, 92%, 93%, 94%, 95%, 96%, 97%, 98%, or 99% sequence identity to SEQ ID NO:41.

27. The complex of any one of claims 16-26, wherein the engineered IL-15 polypeptide and the second polypeptide are linked non-covalently.

28. The complex of any one of claims 16-26, wherein the engineered IL-15 polypeptide and the second polypeptide are linked covalently.

29. The complex of any one of claims 16-28, wherein the complex further comprises a phthalocyanine dye.

30. The complex of claim 29, wherein the phthalocyanine dye is covalently linked to the second polypeptide.

31. A nucleic acid molecule encoding the engineered IL-15 polypeptide of any one of claims 1-15, or the complex of any one of claims 16-30.

32. A vector comprising the nucleic acid molecule of claim 31.

33. The vector of claim 32, wherein the vector is an expression vector.

34. The vector of claim 32 or 33, wherein the vector is a mammalian vector or a viral vector.

35. A cell comprising the engineered IL-15 polypeptide of any one of claims 1-15 or the complex of any one of claims 16-30.

36. A cell comprising the nucleic acid molecule of claim 31 or the vector of any one of claims 32-34.

37. The cell of claim 27 or 28, wherein the cell is a mammalian cell.

38. A pharmaceutical composition comprising the engineered IL-15 polypeptide of any one of claims 1-15, or the complex of any one of claims 16-30.

39. A method for treating a disease or condition, the method comprising administering the engineered IL-15 polypeptide of any one of claims 1-15 in conjunction with an IL-15 receptor or functional domain thereof.

40. The method for claim 39, wherein the engineered IL-15 polypeptide is administered in conjunction with a second polypeptide comprising an IL-15 receptor molecule or domain thereof fused to an Fc domain or a portion thereof.

41. The method for claim 40, wherein the second polypeptide comprises an IL-15 sushi domain fused to an Fc domain.

42. The method for claim 40 or 41, wherein the second polypeptide comprises the amino acid sequence set forth in SEQ ID NO:41, or an amino acid sequence that has at least 90%, 91%, 92%, 93%, 94%, 95%, 96%, 97%, 98%, or 99% sequence identity to SEQ ID NO:41.

43. A method for treating a disease or condition, the method comprising administering the engineered IL-15 polypeptide of any one of claims 1-15, the complex of any one of claims 16-30, or the pharmaceutical composition of claim 38.

44. The method of any one of claims 39-43, wherein the method further comprises administering a second agent.

45. The method for claim 34, wherein the second agent is selected from radiation, photoimmunotherapy, chemotherapy, an immune checkpoint inhibitor, a tyrosine kinase inhibitor, a CAR-T cell, or a CAR-NK cell.

46. A method for treating a disease or condition in a subject, the method comprising administering the engineered IL-15 polypeptide of any one of claims 1-15, and a photoimmunotherapy.

47. A method for treating a disease or condition in a subject, the method comprising administering the complex of any one of claims 16-30, and a photoimmunotherapy.

48. A method for treating a disease or condition in a subject, the method comprising administering the pharmaceutical composition of claim 38, and a photoimmunotherapy.

49. The method for any one of claims 45-48, wherein the photoimmunotherapy comprises:
(a) intravenously administering to the subject a targeting conjugate comprising a silicon phthalocyanine dye linked to a targeting molecule, wherein the targeting molecule is capable of binding to a target on the surface of a target cell; and
(b) after administering the targeting conjugate, irradiating an area around or near a target cell at a wavelength and dose sufficient to kill the target cell, thereby treating the disease or condition.

50. The method for any one of claims 45-49, wherein the engineered IL-15 polypeptide, the complex or the pharmaceutical composition is administered prior to, concurrent with or subsequent to the photoimmunotherapy.

51. The method for claim 49 or 50, wherein the target cell is a tumor cell, a cell present in the tumor microenvironment, or an immune cell.

52. The method for any one of claims 49-51, wherein the targeting molecule is capable of binding to Treg cells.

53. The method for any one of claims 49-51, wherein the targeting molecule is capable of binding to PD-L1 or PD-1.

54. The method for any one of claims 49-51, wherein the targeting molecule is capable of binding to EGFR.

55. A method for modulating an immune response in a subject, comprising administering the engineered IL-15 polypeptide of any one of claims 1-15, the complex of any one of claims 16-30, or the pharmaceutical composition of claim 38 to a subject.

56. The method for claim 55, wherein modulating the immune response treats a disease or condition in the subject.

57. The method for any one of claims 39-54 and 56, wherein the disease or condition is selected from the group consisting of a cancer, a tumor, an infection, a viral infection, an immunocompromised state, and an immune deficiency.

58. The method for claim 55, wherein modulating the immune response increases the immune response to vaccination.

59. The method for any one of claims 55-58, wherein the immune response is an increase is one or more immune modulating molecules in the treated subject as compared to prior to the treatment.
